SEQUENCE LISTING (154 sequences)

SEQ ID NO: 1 - protein, 547 aa, Aquilaria crassna
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIVGITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 2 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaattgtcggtatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttcattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttcaccagagttttggaagtcatgtatgacaaaggtgatggttactctgattctcaaggtgtcacaaaggatagaatcgccgctttacttagacacgcaattgaaatc

SEQ ID NO: 3 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 4 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 5 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRNALHIPYHRNVQRLEARQYIPQYDAEQTKIESLSLFAKIDFNMLQALHQSELREASRWWKEFDFPSKLPYARDRIAEGYYWMMGAHFEPKFSLSRKFLHRIVGITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 6 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 7 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 8 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 9 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFQITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 10 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 11 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFQITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLGILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 12 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 13 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 14 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIMIITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKIREMIEIEWKRLNKTTLEADEISSSVVIPSLNLTRVLEVMYDKGDGYSDSQGVTKDRIAALLRHAIEI

SEQ ID NO: 15 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 16 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 17 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 18 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRVFIITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNFTRVLEVMYDKGDGYSDSQGVTKDRIAALLRHAIEI

SEQ ID NO: 19 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 20 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 21 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 22 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 23 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 24 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFQITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 25 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRVFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 26 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFNITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 27 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 28 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 29 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRVFQITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLGILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 30 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLGILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 31 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 32 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFGITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 33 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 34 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 35 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 36 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNFTRVLEVMYDKGDGYSDSQGVTKDRIAALLRHAIEI

SEQ ID NO: 37 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFIITSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 38 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNFTRVLEVMYDKGDGYSDSQGVTKDRIAALLRHAIEI

SEQ ID NO: 39 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 40 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttaatatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacctcaccagagttttggaagtcatgtatgacaaaggtgatggttactctgattctcaaggtgtcacaaaggatagaatcgccgctttacttagacacgcaattgaaatc

SEQ ID NO: 41 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttcagatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 42 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagaaatgctctgcatattccttaccacagaaatgtacaaagattggaggctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaattgtcggtatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 43 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagagtttttcagatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttaaccagagttttggaagtcatgtatgacaaaggtgatggttactctgattctcaaggtgtcacaaaggatagaatcgccgctttacttagacacgcaattgaaatc

SEQ ID NO: 44 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttggtatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgcttattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacctaaccagagttttggaagtcatgtatgacaaaggtgatggttactctgattctcaaggtgtcacaaaggatagaatcgccgctttacttagacacgcaattgaaatc

SEQ ID NO: 45 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttaatatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacctcaccagagttttggaagtcatgtatgacaaaggtgatggttactctgattctcaaggtgtcacaaaggatagaatcgccgctttacttagacacgcaattgaaatc

SEQ ID NO: 46 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaatttttcagatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttcaccagagttttggaagtcatgtatgacaaaggtgatggttactctgattctcaaggtgtcacaaaggatagaatcgccgctttacttagacacgcaattgaaatc

SEQ ID NO: 47 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttataatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaggcattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 48 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaatttttcagatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctaggcattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 49 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttattatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgctaggcattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 50 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaatttttggtatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttaaccagagttttggaagtcatgtatgacaaaggtgatggttactctgattctcaaggtgtcacaaaggatagaatcgccgctttacttagacacgcaattgaaatc

SEQ ID NO: 51 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaattatgataatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatacgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 52 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtatttcagatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttcaccagagttttggaagtcatgtatgacaaaggtgatggttactctgattctcaaggtgtcacaaaggatagaatcgccgctttacttagacacgcaattgaaatc

SEQ ID NO: 53 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaatttttcagatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgcttattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 54 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaatttttggtatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 55 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagagtgtttataatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttcaccagagttttggaagtcatgtatgacaaaggtgatggttactctgattctcaaggtgtcacaaaggatagaatcgccgctttacttagacacgcaattgaaatc

SEQ ID NO: 56 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaatttttataatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaggcattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacctcaccagagttttggaagtcatgtatgacaaaggtgatggttactctgattctcaaggtgtcacaaaggatagaatcgccgctttacttagacacgcaattgaaatc

SEQ ID NO: 57 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttataatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgctcattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacctgaccagagttttggaagtcatgtatgacaaaggtgatggttactctgattctcaaggtgtcacaaaggatagaatcgccgctttacttagacacgcaattgaaatc

SEQ ID NO: 58 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttataatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctgattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 59 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttcagatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgctgggcattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 60 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttaatatcgtttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctcattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttcaccagagttttggaagtcatgtatgacaaaggtgatggttactctgattctcaaggtgtcacaaaggatagaatcgccgctttacttagacacgcaattgaaatc

SEQ ID NO: 61 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttcagatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttaaccagagttttggaagtcatgtatgacaaaggtgatggttactctgattctcaaggtgtcacaaaggatagaatcgccgctttacttagacacgcaattgaaatc

SEQ ID NO: 62 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagagtgtttaatatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacctaaccagagttttggaagtcatgtatgacaaaggtgatggttactctgattctcaaggtgtcacaaaggatagaatcgccgctttacttagacacgcaattgaaatc

SEQ ID NO: 63 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttaatatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacctaaccagagttttggaagtcatgtatgacaaaggtgatggttactctgattctcaaggtgtcacaaaggatagaatcgccgctttacttagacacgcaattgaaatc

SEQ ID NO: 64 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttataatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaggcattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacctgaccagagttttggaagtcatgtatgacaaaggtgatggttactctgattctcaaggtgtcacaaaggatagaatcgccgctttacttagacacgcaattgaaatc

SEQ ID NO: 65 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttaatatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 66 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagagtgtttcagatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctaggcattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 67 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagagtgtttcagatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttagggattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacctgaccagagttttggaagtcatgtatgacaaaggtgatggttactctgattctcaaggtgtcacaaaggatagaatcgccgctttacttagacacgcaattgaaatc

SEQ ID NO: 68 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttggtatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgctaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 69 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttggtatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgctcattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 70 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagaatttttaatatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacctgaccagagttttggaagtcatgtatgacaaaggtgatggttactctgattctcaaggtgtcacaaaggatagaatcgccgctttacttagacacgcaattgaaatc

SEQ ID NO: 71 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttaatatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttagggattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttaaccagagttttggaagtcatgtatgacaaaggtgatggttactctgattctcaaggtgtcacaaaggatagaatcgccgctttacttagacacgcaattgaaatc

SEQ ID NO: 72 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttataatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgcttattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacttaaccagagttttggaagtcatgtatgacaaaggtgatggttactctgattctcaaggtgtcacaaaggatagaatcgccgctttacttagacacgcaattgaaatc

SEQ ID NO: 73 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagagtgtttcagatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgctaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttcaccagagttttggaagtcatgtatgacaaaggtgatggttactctgattctcaaggtgtcacaaaggatagaatcgccgctttacttagacacgcaattgaaatc

SEQ ID NO: 74 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttataatcacttcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttagggattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttaaccagagttttggaagtcatgtatgacaaaggtgatggttactctgattctcaaggtgtcacaaaggatagaatcgccgctttacttagacacgcaattgaaatc

SEQ ID NO: 75 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaatttttcagatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttactggatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttcaccagagttttggaagtcatgtatgacaaaggtgatggttactctgattctcaaggtgtcacaaaggatagaatcgccgctttacttagacacgcaattgaaatc

SEQ ID NO: 76 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgaatagagtgtttcagatcgtatcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtatggccatctctcaacttaaccagagttttggaagtcatgtatgacaaaggtgatggttactctgattctcaaggtgtcacaaaggatagaatcgccgctttacttagacacgcaattgaaatc

SEQ ID NO: 77 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFNIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 78 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 79 - protein, 547 aa, synthetic construct
MSSAKLGSASEDVSRRDANYHPTVWGDFFLTHSSNFLENNDSILEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKETLEADEISSSVVIPSLNLTRVLEVMYDKGDGYSDSQGVTKDRIAALLRHAIEI

SEQ ID NO: 80 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 81 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 82 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEASHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEEFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 83 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNKIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 84 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFNIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 85 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLNRIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 86 - protein, 547 aa, synthetic construct
MSSAKLGSASEDVSRRDANYHPTVWGDFFLTHSSNFLENNDSILEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFIIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLGILMLIGRGEFELKETLDWASTIPEMVKASSLIARLIDDLQTYKAEEERGETVSAVRCYMREFGVSEEQACKKMREMIEIEWKRLNKTTLEADEISSSVVWPSLNLTRVLEVMYDKGDGYSDSQGVTKDRIAALLRHAIEI

SEQ ID NO: 87 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 88 - protein, 547 aa, synthetic construct
MSSAKLGSASEDVSRRDANYHPTVWGDFFLTHSSNFLENNDSILEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIFGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKETLEADEISSSVVIPSLNLTRVLEVMYDKGDGYSDSQGVTKDRIAALLRHAIEI

SEQ ID NO: 89 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIVGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 90 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMSAHFEPKFSLSRKFLHRIVGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 91 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNIQRLAARQYIPQYDAEQTKIESLSLFAKIDFNMLQALHQSELREASRWWKEFDFPSKLPYARDRIAEGYYWMMGAHFEPKFSLSRKFLHRIVGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 92 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRIVGIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSCISGGYKMLIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 93 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagaatctttaatattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggttgattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 94 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagagtttttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggttgattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 95 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagagtttttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaaggaaacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 96 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatctcttctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagagtttttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 97 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatctcttctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagagtttttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcttggaggtcatgtacgataaaggcgatggttattccgattctcaaggtgtaacaaaggacagaattgctgctttgttaagacacgctatcgaaatt

SEQ ID NO: 98 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagcctctcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagagtttttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagaattcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 99 - DNA, 1643 bp, synthetic construct
  source 1..1643: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaataaaatctttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaattat

SEQ ID NO: 100 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttcaatatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagagtttttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 101 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatctcttctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtctggccaagcttgaacttgaccagagtcttggaggtcatgtacgataaaggcgatggttattccgattctcaaggtgtaacaaaggacagaattgctgctttgttaagacacgctatcgaaatt

SEQ ID NO: 102 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagaatctttattattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgttgggtattttgatgcttatcggtagaggtgaatttgaattaaaagaaaccttggattgggcttctaccatcccagaaatggtcaaggcttcctctttgattgctaggttgattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtctggccaagcttgaacttgaccagagtcttggaggtcatgtacgataaaggcgatggttattccgattctcaaggtgtaacaaaggacagaattgctgctttgttaagacacgctatcgaaatt

SEQ ID NO: 103 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagaatctttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggttgattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 104 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagaatctttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaaggaaacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 105 - DNA, 1641 bp, synthetic construct
  source 1..1641: mol_type = other DNA
atgtcttcagctaaactgggaagtgcctccgaggacgtttctcgtcgggatgcaaattaccaccctactgtatggggtgatttctttttgacgcatagctccaacttcctcgaaaacaatgactcgatccttgaaaagcacgaagaattgaagcaggaagtgaggaacctattagtcgttgaaacatctgacttgccatctaaaattcaattgaccgatgagataattagattgggtgttggctatcattttgaaactgaaatcaaggctcaattagaaaagttgcacgaccaccaattgcatttaaacttcgatcttttgaccacttcggtctggttcagacttttgcgaggtcacggtttttccattccatcagatgtttttaagagattcaaaaacaccaagggtgaatttgagactgaagacgcgagaacattatggtgtttgtacgaagccacccatcttagagttgacggggaggatatcctagaagaggctatacaattctctcgtaagagattggaagctctgttacccaaattgagcttcccattgtccgaatgcgtcagagatgctctgcatattccttaccacagaaatgtacaaagattggccgctagacaatatatcccacaatacgatgctgaacagactaagattgaatctctatctttattcgccaagatcgactttaacatgttgcaagctttgcaccaaagtgaactgagagaagcctcacgctggtggaaagaatttgacttcccatccaagttaccttacgcaagggatagaattgctgaaggttattactggatgatgggtgctcattttgaaccgaagttctctttgtctcgtaagttcttgcatagaattgtcggtatcgtgtcgttaattgatgacacgtatgatgtttacgggacccttgaagaggtgacactattcactgaagcggtcgaacgttgggacattgaggcagttaaagatatcccaaagtacatgcaagtcatctacatcggtatgttgggtatctttgaagactttaaagataacttaattaatgctcgaggtaaagattactgtatcgactatgctattgaagtctttaaggaaattgttcgttcctatcaaagagaagctgaatatttccacaccggctacgttccaagttacgatgaatacatggagaatagtataatatctggaggttacaagatgttaattattcttatgttgattggtagaggggaatttgaattgaaggaaacactagactgggcctcaactattcctgaaatggttaaggctagttctctcattgctagatacatcgacgatctccagacctataaggcagaggaggaaagaggtgaaactgtatcagctgttagatgttacatgagagaatttggtgtgtctgaggaacaagcttgcaagaaaatgcgtgaaatgatcgaaattgaatggaagcgattgaacaagacgactttggaggcagacgaaatatcctccagtgttgtaataccatctctcaacctcaccagagttttggaagtcatgtatgacaaaggtgatggttactctgattctcaaggtgtcacaaaggatagaatcgccgctttacttagacacgcaattgaaatc

SEQ ID NO: 106 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgtctgcccacttcgaaccaaagttctccttatctagaaaatttttgcatagaatcgttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 107 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatattcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagaatcgttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 108 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagaatcgttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgtgtatttctggtggttataaaatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 109 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcatagaatctttggtattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 110 - protein, 547 aa, synthetic construct
MSSAKLGSASEDVSRRDANYHPTVWGDFFLTHSSNFLENNDSILEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHKVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARLIDDLQTYKAEEERGETVSAVRCYMREFGVSEEQACKKMREMIEIEWKRLNKETLEADEISSSVVIPSLNLTRVLEVMYDKGDGYSDSQGVTKDRIAALLRHAIEI

SEQ ID NO: 111 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 112 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFEEEIKAQLEKLHDHQLHLNFDLLTTSVWFRLLRQHGFSIPSDVFKRFKNDKGEFETEDARTLLCLYEATHLRVDGEDILEEAIQFSRKRLEALLPKLSFPLSECVRDALHIPYHRNVQRLAARQYIPFYDAEETKIESLLLFAKIDFNMLQALHQSELREASRWWKELDFASKLPYARDRIAEGYYWMMGAHFEPKFSLSRKFLHKVFQIVSLIDDTYDVYGTLEEVTLFTEAVERWDIEAVKDIPKYMQVIYIGMLGIFEEFKDNLINARGKDYCIDYAIEVFKEIVRSYQREAEYFHTGYVPSFDEYMENSIISGGYWMLIILMLIGRGEFESKETLDWASTIPEMVKASSLIARLIDDLQTYKAEEERGEVVSAVRCYMKEFGVSEEQACKKMREMIEIEWKRLNKTTLEADEISSSVVIPSLNLTRVLEVMYDKGDGYSDSQGVTKDRIAALLVHAIEI

SEQ ID NO: 113 - protein, 547 aa, synthetic construct
MSSAKLGSASEDVSRRDANYHPDVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNVQRLAARQYIPQYDAEQTKIESLSLFAKIDFNMLQALHQSELREASRWWKEFDFPSKLPYARDRIAEGYYWMMGAHFEPKFSLSRKFLHRVFQIVSLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYWMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNLTRVLEVMYDKGDGYSDSQGVTKDRIAALLRHAIEI

SEQ ID NO: 114 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLHRVFNIVSLIDDTYDVYGTLEEVTLFTEAVERWDIEAVKDIPKYMQVIYIGMLGIFEDFKDNLINARGKDYCIDYAIEVFKEIVRSYQREAEWFHTGYVPSYDEYMENSIISGGYWMLIILMLIGRGEFELKETLDWASTIPEMVKASSLIARYIDDLQTYKAEEERGETVSAVRCYMREFGVSEEQACKKMREMIEIEWKRLNKETLEADEISSSVVIPSLNLTRVLEVMYDKGDGYSDSQGVTKDRIAALLRHAIEI

SEQ ID NO: 115 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 116 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRVVEVMYDKGDGYSDSQGVTKDRIAALLRHAIEI

SEQ ID NO: 117 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRVVEVMYDKGDGYSDSQGVTKDRIAALLRHAIEI

SEQ ID NO: 118 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRVAEVMYDKGDGYSDSQGVTKDRIAALLRHAIEI

SEQ ID NO: 119 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYWNDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 120 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRVLEVLYDKGDGYSDSQGVTKDRIAALLRHAIEI

SEQ ID NO: 121 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLVCLYEATHLRVDGEDILEEAIQFSRKRLEALLPKLSFPLSEQVRDALHIPYHRNSQRLAARQYIPQYDAEQTKIESLLLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 122 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSLWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLLLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYTNDLQGYKAEEERGETVSAVRCYMREFGVSEEQACKKMREMIEIEWKRLNKTTLEADEISSSVVIPSLNFTRVLEVLYDKGDGYSDSQGVTKDRIAALLRHAIEI

SEQ ID NO: 123 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSLWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMIKASSLIARYTNDLQGYKAEEERGETVSAVRCYMREFGVSEEQACKKMREMIEIEWKRLNKTTLEANEISSSVVIPSLNFTRVVEVMYDKGDGYSDSQGVTKDRIAALLRHAIEI

SEQ ID NO: 124 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRVVEVLYDKGDGYSDSQGVTKDRIAALLRHAIEI

SEQ ID NO: 125 - protein, 547 aa, synthetic construct
MSSAKLGSASEDVSRRDANYHPTVWGDFFLTHSSNFLENNDSIVEKHEELKQEVRNLLVVETSDLPSKIQLIDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGETVSAVRCYMREFGVSEEQACKKMREMIEIEWKRLNKTTLEADEISSSVVIPSLNFTRMVEVMYDKGDGYSDSQGVTKDRIAALLRHAIEI

SEQ ID NO: 126 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSLWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLLLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYTNDLQTYKAEEERGETVSAVRCYMREFGVSEEQACKKMREMIEIEWKRLNKTTLEADEISSSVVIPSLNFTRVVEVMYDKGDGYSDSQGVTKDRIAALLRHAIEI

SEQ ID NO: 127 - protein, 547 aa, synthetic construct; identical to SEQ ID NO: 116

SEQ ID NO: 128 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYINDLQTYKAEEERGELVSAVRCYMREFGVSEEQACKKMREMIEIEWKRLNKTTLEADEISSSVVIPSLNFTRVAEVMYDKGDGYSDSQGVTKDRIAALLRHAIEI

SEQ ID NO: 129 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYTNDLQTYKAEEERGETVSAVRCYMREFGVSEEQACKKMREMIEIEWKRLNKTTLEADEISSSVVIPSLNFTRVVEVMYDKGDGYSDSQGVTKDRIAALLRHAIEI

SEQ ID NO: 130 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMIKASSLIARYINDLQTYKAEEERGETVSAVRCYMREFGVSEEQACKKMREMIEIEWKRLNKTTLEADEISSSVVIPSLNFTRVVEVMYDKGDGYSDSQGVTKDRIAALLRHAIEI

SEQ ID NO: 131 - protein, 547 aa, synthetic construct
MSSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSIPSDVFKRFKNTKGEFETEDARTLWCLYEATHLRVDGEDILEEAIQFSRKRLEALLPKLSFPLSECVRDALHIPYHRNSQRLAARQYIPQYDAEQTKIESLSLFAKIDFNMLQALHQSELREASRWWKEFDFPSKLPYARDRIAEGYYWMMGAHFEPKFSLSRKFLNRICGIISLIDDTYDVYGTLEEVTLFTEAVERWDIEAVKDIPKYMQVIYIGMLGIFEDFKDNLINARGKDYCIDYAIEVFKEIVRSYQREAEYFHTGYVPSYDEYMENSIISGGYKMFIILMLIGRGEFELKETLDWASTIPEMLKASSLIARYINDLQTYKAEEERGETVSAVRCYMREFGVSEEQACKKMREMIEIEWKRLNKTTLEADEISSSVVIPSLNFTRVVEVMYDKGDGYSDSQGVTKDRIAALLRHAIEI

SEQ ID NO: 132 - skipped

SEQ ID NO: 133 - DNA, 1641 bp, synthetic construct
  source 1..1641: mol_type = other DNA
atgtcatctgctaagttaggttctgctagcgaagatgtttccagaagggacgcaaattaccatcccactgtctggggcgactttttcttaacgcattcttcgaactttctggagaacaacgactctattttggaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctcgttggtggaaggagttcgatttcccttctaagttaccatacgccagagacagaattgctgagggttattactggatgatgggtgcccacttcgaaccaaagttctccttatctcgtaaatttttgcataaggtgtttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgttaattattttgatgcttatcggtagaggtgaatttgaattaaaagaaaccttggattgggcttctaccatcccagaaatggtcaaggcttcctctttgattgctaggttgattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaaggaaacccttgaagctgacgaaatttcctcatccgtagtcatcccaagcttgaacttgaccagagtcttggaggtcatgtacgataaaggcgatggttattccgattctcaaggtgtaacaaaggacagaattgctgctttgttaagacacgctatcgaaatt

SEQ ID NO: 134 - DNA, 1641 bp, synthetic construct
  source 1..1641: mol_type = other DNA
atgtcatctgctaagttaggttctgctagcgaagatgtttccagaagggacgcaaattaccatcccactgtctggggcgactttttcttaacgcattcttcgaactttctggagaacaacgactctattgttgaaaagcatgaggagttgaagcaagaagttagaaacttgcttgtggtcgaaacctccgacttaccaagtaagatacaattaatcgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctcgttggtggaaggagttcgatttcccttctaagttaccatacgccagagacagaattgctgagggttattactggatgatgggtgcccacttcgaaccaaagttctccttatctcgtaaatttttgcacagagttttccaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgctaattattttgatgcttatcggtagaggtgaatttgaattaaaagaaaccttggattgggcttctaccatcccagaaatggtcaaggcttcctctttgattgctaggttgattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacctaaccagagtcttggaggtcatgtacgataaaggcgatggttattccgattctcaaggtgtaacaaaggacagaattgctgctttgttaagacacgctatcgaaatt

SEQ ID NO: 135 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgttgaaaagcatgaggagttgaagcaagaagttagaaacttgcttgtggtcgaaacctccgacttaccaagtaagatacaattaattgatgaaattattagattgggtgtcggttaccactttgaagaagagatcaaggcacaattagaaaagttgcacgaccatcaattacatttgaattttgatttgttgactacaagcgtatggtttagactattgcgtcaacacggtttctctatcccatctgacgtttttaagagatttaagaacgataagggtgagttcgaaacagaagatgcaagaaccctattatgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctttctacgatgctgaagagactaaaattgagtctctattgctatttgccaagatcgactttaatatgttacaagcactgcaccagtctgaactaagagaagcctctagatggtggaaggaattggatttcgcctctaagttaccatacgccagagacagaattgctgagggttattactggatgatgggtgcccacttcgaaccaaagttctccttatctagaaaatttttgcataaggtatttcaaattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagaattcaaggataacttgattaatgcaagaggaaaagactattgcattgactatgctatcgaagtttttaaagaaattgtcaggtcgtaccaaagagaagctgagtatttccacactggatacgtcccatcattcgacgagtacatggaaaactcgattatttctggtggttattggatgttgattattttgatgcttatcggtagaggtgaatttgaatctaaagaaaccttggattgggcttctaccatcccagaaatggtcaaggcttcctctttgattgctaggttgattgacgacttgcaaacctacaaggccgaggaagaaagaggtgaagtcgtctccgctgttagatgttatatgaaggagttcggtgttagtgaagaacaagcttgtaagaaaatgcgtgaaatgatcgaaattgaatggaagagactaaacaagactacccttgaagctgacgaaatttcctcatccgtagtcatcccaagcttgaaccttaccagagtcttggaggtcatgtacgataaaggcgatggttattccgattctcaaggtgtaacaaaggacagaattgctgctttgttagttcacgctatcgaaatt

SEQ ID NO: 136 - DNA, 1641 bp, synthetic construct
  source 1..1641: mol_type = other DNA
atgtcatctgctaagttaggttctgctagcgaagatgtttctagaagggacgcaaattaccatcccgatgtctggggcgatttttttttgacgcattcttcgaactttctggagaacaacgactctattgttgaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatgttcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgcacagagtgttccagattgtttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttattggatgctgattattttgatgcttatcggtagaggtgaatttgaattaaaagaaaccttggattgggcttctaccatcccagaaatggtcaaggcttcctctttgattgctaggttgattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaacttaaccagagtcttggaggtcatgtacgataaaggcgatggttattccgattctcaaggtgtaacaaaggacagaattgctgctttgttaagacacgctatcgaaatt

SEQ ID NO: 137 - DNA, 1641 bp, synthetic construct
  source 1..1641: mol_type = other DNA
atgtcatctgctaagttaggttctgctagcgaagatgtttccagaagggacgcaaattaccatcccactgtctggggcgactttttcttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccatcgtaactctcaaagattggctgccagacaatacattcctcaatacgatgctgaacaaactaaaattgagtctctatctctatttgccaagatcgactttaatatgttacaagcactgcaccagtctgaactaagagaagcctcgagatggtggaaggaatttgatttcccttctaagttaccatacgccagagacagaattgctgagggttattactggatgatgggtgcccacttcgaaccaaagttctccttatccaggaaatttttgcacagagtctttaatattgtatcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtggttccacactggatacgtcccatcatacgacgagtacatggaaaactcgattatttctggtggttattggatgttgattattttgatgcttatcggtagaggtgaatttgaattaaaagaaaccttggattgggcttctaccatcccagaaatggtcaaggcttcctctttgattgctaggtacattgacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaaggaaacccttgaagctgacgaaatttcctcatccgtagtcatcccaagcttgaacttaaccagagtcttggaggtcatgtacgataaaggcgatggttattccgattctcaaggtgtaacaaaggacagaattgctgctttgttaagacacgctatcgaaatt

SEQ ID NO: 138 - DNA, 1641 bp, synthetic construct
  source 1..1641: mol_type = other DNA
atgtcatctgctaagttaggttctgctagcgaagatgtttccagaagggacgcaaattaccatcccactgtctggggcgactttttcttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccatagaaacagccaaagattggctgccagacaatacattcctcaatacgatgctgaacaaactaaaattgagtctctatctctatttgccaagatcgactttaatatgttacaagcactgcaccagtctgaactaagagaagcctcgagatggtggaaggaatttgatttcccttctaagttaccatacgccagagacagaattgctgagggttattactggatgatgggtgcccacttcgaaccaaagttctccttatccaggaaatttttgaatagaatctgtggtattatctcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaatgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcttggaggtcatgtacgataaaggcgatggttattccgattctcaaggtgtaacaaaggacagaattgctgctttgttaagacacgctatcgaaatt

SEQ ID NO: 139 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcccaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgcggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgttgaggtcatgtacgataaaggcgatggttattccgattctcaaggtgtaacaaaggacagaattgctgctttgttaagacacgctatcgaaatt

SEQ ID NO: 140 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgtcgaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcccaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgttgaggtcatgtacgataaaggcgatggttattccgattctcaaggtgtaacaaaggacagaattgctgctttgttaagacacgctatcgaaatt

SEQ ID NO: 141 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgtcgaaaagcatgaggagttgaagcaagaagttagaaacttgcttgtggtcgaaacctccgacttaccaagtaagatacaattaatcgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattctcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatctcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgctgaggtcatgtacgataaaggcgatggttattccgattctcaaggtgtaacaaaggacagaattgctgctttgttaagacacgctatcgaaatt

SEQ ID NO: 142 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgttgaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcacaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatctcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtactggaatgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcttggaggtcatgtacgataaaggcgatggttattccgattctcaaggtgtaacaaaggacagaattgctgctttgttaagacacgctatcgaaatt

SEQ ID NO: 143 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgtcgaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcccaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcttggaggtcttgtacgataaaggcgatggttattccgattctcaaggtgtaacaaaggacagaattgctgctttgttaagacacgctatcgaaatt

SEQ ID NO: 144 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctagtctgtttatatgaagccacgcatttgagagtcgatggagaggacatccttgaggaagccattcaattctccagaaaaagacttgaggcattgctgcctaaattgtcttttcctttgtctgaacaagtcagggatgcattacacatcccctaccataggaattcacaaagattggctgccagacaatacattcctcaatacgatgctgaacaaactaaaattgagtctctattgctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcttggaggtcatgtacgataaaggcgatggttattccgattctcaaggtgtaacaaaggacagaattgctgctttgttaagacacgctatcgaaatt

SEQ ID NO: 145 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgttgaaaagcatgaggagttgaagcaagaagttagaaacttgcttgtggtcgaaacctccgacttaccaagtaagatacaattaatcgatgaaattattagattgggtgtcggttaccactttgaaactgagatcaaggcacaattagaaaagttgcacgaccatcaattacatttgaattttgatttgttgactacaagcttgtggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcacaaagattggctgccagacaatacattcctcaatacgatgctgaacaaactaaaattgagtctctattgctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacactaatgacttgcaaggttacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcttggaggtcttgtacgataaaggcgatggttattccgattctcaaggtgtaacaaaggacagaattgctgctttgttaagacacgctatcgaaatt

SEQ ID NO: 146 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgtcgaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcttgtggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcccaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgcggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatgattaaggcttcctctttgattgctaggtacactaacgacttgcaaggttacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctaacgaaatttcctcatccgtagtcatcccaagcttgaactttaccagagtcgttgaggtcatgtacgataaaggcgatggttattccgattctcaaggtgtaacaaaggacagaattgctgctttgttaagacacgctatcgaaatt

SEQ ID NO: 147 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgttgaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcccaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatatcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaatgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgtcgaggtcttatacgataaaggcgatggttattccgattctcaaggtgtaacaaaggacagaattgctgctttgttaagacacgctatcgaaatt

SEQ ID NO: 148 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattgttgaaaagcatgaggagttgaagcaagaagttagaaacttgcttgtggtcgaaacctccgacttaccaagtaagatacaattaattgatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcccaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatctcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagaatggtggaggtcatgtacgataaaggcgatggttattccgattctcaaggtgtaacaaaggacagaattgctgctttgttaagacacgctatcgaaatt

SEQ ID NO: 149 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcttatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattctcaaagattggctgccagacaatacattcctcaatacgatgctgaacaaactaaaattgagtctctactgctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacactaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgtcgaggtcatgtacgataaaggcgatggttattccgattctcaaggtgtaacaaaggacagaattgctgctttgttaagacacgctatcgaaatt

SEQ ID NO: 150 - DNA, 1641 bp, synthetic construct
  source 1..1641: mol_type = other DNA
atgtcctcagctaaattgggtagcgcttccgaagacgtaagcagaagagatgctaactaccatcctaccgtttggggtgacttcttcttgactcactcttcaaactttttagaaaacaacgactctatcttggaaaagcacgaggaattgaagcaagaagttagaaatttgttggtagtggaaacctcggacttgccatccaagattcaattgacggatgaaattattaggttgggtgtaggttaccacttcgagactgaaataaaagctcaattagaaaaattgcatgaccaccaactacacttgaacttcgacttgctaaccacttccgtttggtttagattacttaggggtcacggtttctctattccaagcgatgtcttcaaaagattcaagaacacaaaaggtgaatttgaaactgaagatgctagaaccttgtggtgcttatatgaagcaactcacttgagagtcgatggtgaagatatcctagaagaagccatccaattctcgaggaagagacttgaagctttattgccaaagttgtctttccctttgtccgaatgcgtgagagatgccctacatataccataccacagaaactctcaacgtttggctgctagacaatatatcccacaatatgacgctgaacaaactaagatcgaatcactatctctattcgcaaagattgatttcaatatgttgcaagccttacaccaatctgagcttagagaggcctcaagatggtggaaggaatttgatttcccatccaaattgccatacgctagagacagaatcgctgaaggttactattggatgatgggtgcccatttcgaaccaaagttttctttatcaagaaagtttctgaatagaatctgtggtatcatttccttgattgatgacacttacgacgtttacggtactttggaggaagttaccttgttcaccgaagctgttgaacgttgggatatcgaagcagttaaggacatcccaaagtacatgcaagttatatacatcggaatgttaggtatcttcgaagatttcaaggataatcttatcaacgcaagaggtaaagattactgtattgattacgctatcgaagtttttaaggaaatagtcagatcataccaaagagaagccgagtacttccatactggttatgttccatcatacgatgaatatatggagaacagcataatttctggtggttataagatgttcatcatcttaatgttaattggtagaggtgaatttgagttgaaggaaaccttagactgggcctctactatccccgaaatggtcaaggcaagctccctgatcgctagatacataaatgatttgcaaacctacaaggctgaagaggaaagaggtgaaaccgtttctgctgtcagatgctacatgagagaatttggagtctctgaagagcaagcctgtaagaaaatgagagaaatgattgaaattgaatggaagaggttgaataaaactactttggaggctgacgagatttcctcttctgttgtcatcccatccttgaacttcacaagagttgttgaagtaatgtacgataagggcgacggttactctgactctcaaggcgttaccaaggacagaattgccgctcttttaagacacgccattgagatt

SEQ ID NO: 151 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaatagtcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgcggtattatctcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacattaatgacttgcaaacctacaaggccgaggaagaaagaggtgaattagtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgctgaggtcatgtacgataaaggcgatggttattccgattctcaaggtgtaacaaaggacagaattgctgctttgttaagacacgctatcgaaatt

SEQ ID NO: 152 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcccaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgtggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatggtcaaggcttcctctttgattgctaggtacacgaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgtagaggtcatgtacgataaaggcgatggttattccgattctcaaggtgtaacaaaggacagaattgctgctttgttaagacacgctatcgaaatt

SEQ ID NO: 153 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattcacaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgcggtattatttcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatgatcaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgtcgaggtcatgtacgataaaggcgatggttattccgattctcaaggtgtaacaaaggacagaattgctgctttgttaagacacgctatcgaaatt

SEQ ID NO: 154 - DNA, 1641 bp, synthetic construct
  source 1..1641: mol_type = other DNA
atgtcatctgctaagttaggttctgctagcgaagatgtttctagaagggacgcaaattaccatcccactgtctggggcgatttttttttgacgcattcttcgaactttctggagaacaacgactctattttggaaaagcatgaggagttgaagcaagaagttagaaacttgcttgtggtcgaaacctccgacttaccaagtaagatacaattaacagatgaaattattagattgggtgtcggttaccactttgaaactgagatcaaggcacaattagaaaagttgcacgaccatcaattacatttgaattttgatttgttgactacaagcgtatggtttagactattgcgtggtcacggtttctctatcccatctgacgtttttaagagatttaagaacaccaagggtgagttcgaaacagaagatgcaagaaccctatggtgtttatatgaagccacgcatttgagagtcgatggagaggacatccttgaggaagccattcaattctccagaaaaagacttgaggcattgctgcctaaattgtcttttcctttgtctgaatgtgtcagggatgcattacacatcccctaccataggaattctcaaagattggctgccagacaatacattcctcaatacgatgctgaacaaactaaaattgagtctctatctctatttgccaagatcgactttaatatgttacaagcactgcaccagtctgaactaagagaagcctctagatggtggaaggaattcgatttcccttctaagttaccatacgccagagacagaattgctgagggttattactggatgatgggtgcccacttcgaaccaaagttctccttatctagaaaatttttgaatagaatctgcggtattatctcactaatcgacgatacctatgatgtttacggtaccttggaagaagtgactttgtttactgaagccgtcgaaagatgggatatcgaagctgttaaggacattcccaagtacatgcaagtcatttatattggaatgttaggtatcttcgaagatttcaaggataacttgattaatgcaagaggaaaagactattgcattgactatgctatcgaagtttttaaagaaattgtcaggtcgtaccaaagagaagctgagtatttccacactggatacgtcccatcatacgacgagtacatggaaaactcgattatttctggtggttataaaatgtttattattttgatgcttatcggtagaggtgaatttgaattaaaagaaaccttggattgggcttctaccatcccagaaatgttaaaggcttcctctttgattgctaggtacattaacgacttgcaaacctacaaggccgaggaagaaagaggtgaaactgtctccgctgttagatgttatatgagagagttcggtgttagtgaagaacaagcttgtaagaaaatgcgtgaaatgatcgaaattgaatggaagagactaaacaagactacccttgaagctgacgaaatttcctcatccgtagtcatcccaagcttgaactttaccagagtcgttgaggtcatgtacgataaaggcgatggttattccgattctcaaggtgtaacaaaggacagaattgctgctttgttaagacacgctatcgaaatt

(19) United States
(12) Patent Application Publication
(10) Pub. No.: US 2025/0027070 A1
(43) Pub. Date: Jan. 23, 2025

(54) ENGINEERED SESQUITERPENE SYNTHASES

(71) Applicant: Ginkgo Bioworks, Inc., Boston, MA (US)

(72) Inventors: Jeffrey Ian Boucher, Allston, MA (US); Dylan Alexander Carlin, Jamaica Plain, MA (US); Cinzia Formighieri, Saugus, MA (US); Bryan Greenhagen, Melrose, MA (US); Jaide Jensen, Cambridge, MA (US); Scott Marr, Boston, MA (US)

(73) Assignee: Ginkgo Bioworks, Inc., Boston, MA (US)

(21) Appl. No.: 18/713,132

(22) PCT Filed: Nov. 18, 2022
(86) PCT No.: PCT/US2022/080127

§ 371 Date: May 23, 2024

Related U.S. Application Data
(60) Provisional application No. 63/283,194, filed on Nov. 24, 2021.
(60) Provisional application No. 63/285,468, filed on Dec. 2, 2021.

(51) Int. Cl. C12N 9/88; C12P 5/00

(52) U.S. Cl. CPC C12N 9/88; C12P 5/007; C12Y 402/03

(57) ABSTRACT

Provided in this disclosure are engineered sesquiterpene synthases, host cells expressing engineered sesquiterpene synthases and methods for making alpha-guaiene.

### CROSS-REFERENCE TO RELATED APPLICATIONS

This application claims the benefit under 35 U.S.C. § 119 (e) of U.S. Provisional Application No. 63/283,194, filed Nov. 24, 2021, entitled “ENGINEERED SESQUITERPENE SYNTHASES” and U.S. Provisional Application No. 63/285,468, filed Dec. 2, 2021, entitled “ENGINEERED SESQUITERPENE SYNTHASES,” the entire disclosure of each of which is hereby incorporated by reference in its entirety.

### REFERENCE TO AN ELECTRONIC SEQUENCE LISTING

The contents of the electronic sequence listing (G091970079WO00-SEQ-KVC.xml; Size: 300,953 bytes; and Date of Creation: Nov. 15, 2022) is herein incorporated by reference in its entirety.

### FIELD OF THE INVENTION

The disclosure relates to sesquiterpene synthases, host cells comprising sesquiterpene synthases, and methods for making sesquiterpenes.

### BACKGROUND

Terpenes are a diverse class of organic compounds built from five carbon building blocks and encompass at least 400 distinct structural families. Given their structural diversity, terpenes have numerous roles, including acting as pheromones, anti-oxidants, and anti-microbial agents. Monoterpenes (C10), sesquiterpenes (C15), diterpenes (C20), and triterpenes (C30) make up the majority of terpenes. Guaienes are one type of sesquiterpenes, which are formed from three isoprene units and often have the molecular formula C₁₅H₂₄, include compounds such as alpha-guaiene, delta-guaiene (alpha-bulnesene), aciphyllene, beta-guaiene, gamma-guaiene and gamma-gurjunene, which all have a guaiane sesquiterpene skeleton. Guaienes have been used in numerous settings including in the production of fragrances and flavoring agents. Although guaienes may be extracted from plants, these resources are limited as many of these plants are endangered species. Furthermore, the wide array of sesquiterpene isomers often hinders high yield extractions from naturally occurring sources, while the structural complexity of guaienes often limits de novo chemical synthesis.

### SUMMARY

Aspects of the disclosure relate to host cells that comprise a heterologous polynucleotide encoding a sesquiterpene synthase, wherein the sequence of the sesquiterpene synthase comprises one or more amino acid substitutions relative to SEQ ID NO: 1, and wherein at least one of the amino acid substitutions is at a position corresponding to position 122, 274, 275, 293, 295, 301, 368, 398, 404, 407, 507, 515, 525, and/or 533 in SEQ ID NO: 1. In some embodiments, the sequence of the sesquiterpene synthase further comprises at least one amino acid substitution at a position corresponding to position 72, 124, 153, 191, 201, 205, 289, 290, 291, 292, 346, 406, 442, 480, 494, 509, 512, and/or 526 in SEQ ID NO: 1.

Further aspects of the disclosure relate to host cells that comprise a heterologous polynucleotide encoding a sesquiterpene synthase, wherein the sequence of the sesquiterpene synthase comprises two or more amino acid substitutions relative to SEQ ID NO: 1, wherein: at least one of the amino acid substitutions is at a position corresponding to position 122, 274, 275, 293, 295, 301, 368, 398, 404, 407, 507, 515, 525, and/or 533 in SEQ ID NO: 1; and at least one of the amino acid substitutions is at a position corresponding to position 72, 124, 153, 191, 201, 205, 289, 290, 291, 292, 346, 406, 442, 480, 494, 509, 512, and/or 526 in SEQ ID NO: 1.

In some embodiments, the host cell is capable of producing sesquiterpene products, wherein at least 50% of the total sesquiterpene products produced by the host cell is alpha-guaiene. In some embodiments, at least 15% of the total sesquiterpene products produced by the host cell is aciphyllene.

Further aspects of the disclosure relate to host cells that comprise a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, wherein the two or more amino acid substitutions are at positions corresponding to position 72, 122, 124, 153, 191, 201, 205, 274, 275, 289, 290, 291, 292, 293, 295, 301, 346, 368, 398, 404, 406, 407, 442, 480, 494, 507, 509, 512, 526 and/or 533 in SEQ ID NO: 1, wherein the host cell is capable of producing sesquiterpene products, and wherein at least 50% of the total sesquiterpene products produced by the host cell is alpha-guaiene. In some embodiments, the sesquiterpene synthase comprises 4, 5, 6, 7, 8, 9, or more than 9 amino acid substitutions relative to SEQ ID NO: 1.

In some embodiments, the sesquiterpene synthase comprises: an isoleucine (I) residue at a position corresponding to position 72 in SEQ ID NO: 1; an asparagine (N) residue at a position corresponding to position 122 in SEQ ID NO: 1; a serine(S) residue at a position corresponding to position 124 in SEQ ID NO: 1; an S residue at a position corresponding to position 153 in SEQ ID NO: 1; an N residue at a position corresponding to position 191 in SEQ ID NO: 1; an I residue at a position corresponding to position 201 of SEQ ID NO: 1; a glutamate (E) residue at a position corresponding to position 205 in SEQ ID NO: 1; an S residue at a position corresponding to position 274 in SEQ ID NO: 1; a glycine (G) residue at a position corresponding to position 275 in SEQ ID NO: 1; a histidine (H) residue at a position corresponding to position 289 in SEQ ID NO: 1; a lysine (K) residue at a position corresponding to position 290 in SEQ ID NO: 1; a valine (V) residue at a position corresponding to position 291 in SEQ ID NO: 1; a phenylalanine (F) or methionine (M) residue at a position corresponding to position 292 in SEQ ID NO: 1; a glutamine (Q), I, or N residue at a position corresponding to position 293 in SEQ ID NO: 1; a V residue at a position corresponding to position 295 in SEQ ID NO: 1; an S residue at a position corresponding to position 301 in SEQ ID NO: 1; an E residue at a position corresponding to position 346 in SEQ ID NO: 1; a cysteine (C) residue at a position corresponding to position 368 in SEQ ID NO: 1; a C residue at a position corresponding to position 398 in SEQ ID NO: 1; a tryptophan (W) residue at a position corresponding to position 404 in SEQ ID NO: 1; a leucine (L) residue at a position corresponding to position 406 in SEQ ID NO: 1; a G residue at a position corresponding to position 407 in SEQ ID NO: 1; an L residue at a position corresponding to position 442 in SEQ ID NO: 1; an I residue at a position corresponding to position 480 in SEQ ID NO: 1; an E residue at a position corresponding to position 494 in SEQ ID NO: 1; a tryptophan (W) residue at a position corresponding to position 507 in SEQ ID NO: 1; an alanine (A) residue at a position corresponding to position 509 in SEQ ID NO: 1; an L residue at a position corresponding to position 512 in SEQ ID NO: 1; an F residue at a position corresponding to position 526 in SEQ ID NO: 1; an N residue at a position corresponding to position 533 in SEQ ID NO: 1; or any combination thereof.

In some embodiments, the sesquiterpene synthase comprises: an I residue at a position corresponding to position 72 in SEQ ID NO: 1; an N residue at a position corresponding to position 122 in SEQ ID NO: 1; an S residue at a position corresponding to position 124 in SEQ ID NO: 1; an S residue at a position corresponding to position 153 in SEQ ID NO: 1; an N residue at a position corresponding to position 191 in SEQ ID NO: 1; an I residue at a position corresponding to position 201 in SEQ ID NO: 1; an E residue at a position corresponding to position 205 in SEQ ID NO: 1; an S residue at a position corresponding to position 274 in SEQ ID NO: 1; a G residue at a position corresponding to position 275 in SEQ ID NO: 1; an L, T, S, H, M or D residue at a position corresponding to position 289 in SEQ ID NO: 1; a K residue at a position corresponding to position 290 in SEQ ID NO: 1; an F, L, T, V or C residue at a position corresponding to position 291 in SEQ ID NO: 1; an A, Q, C, Y, H, E, F, M, W, T or F residue at a position corresponding to position 292 in SEQ ID NO: 1; an L, V, T, Y, C, F, W, Q, I, N, or M residue at a position corresponding to position 293 in SEQ ID NO: 1;an E, D, N, W, G, V or I residue at a position corresponding to position 295 in SEQ ID NO: 1; an S residue at a position corresponding to position 301 in SEQ ID NO: 1; an E residue at a position corresponding to position 346 in SEQ ID NO: 1; a C residue at a position corresponding to position 368 in SEQ ID NO: 1; a C residue at a position corresponding to position 398 in SEQ ID NO: 1; a W residue at a position corresponding to position 404 in SEQ ID NO: 1; an L, N, W, or T residue at a position corresponding to position 406 in SEQ ID NO: 1; a G residue at a position corresponding to position 407 in SEQ ID NO: 1; an L residue at a position corresponding to position 442 in SEQ ID NO: 1; an I residue at a position corresponding to position 480 in SEQ ID NO: 1; an E residue at a position corresponding to position 494 in SEQ ID NO: 1; a W residue at a position corresponding to position 507 in SEQ ID NO: 1; an A residue at a position corresponding to position 509 in SEQ ID NO: 1; an L residue at a position corresponding to position 512 in SEQ ID NO: 1; an F residue at a position corresponding to position 526 of SEQ ID NO: 1; an N residue at a position corresponding to position 533 of SEQ ID NO: 1; or any combination thereof.

In some embodiments, the sesquiterpene synthase comprises the following amino acid substitutions relative to SEQ ID NO: 1: N289H, V292F, G293N, T295V, K404W, F406L, and F512L; N289H, 1291V, V292F, G293Q, T295V, K404W, F406L, and F512L; N289H, F406L, F512L, D19IN, and A205E; 1291V, V292F, G293Q, T295V, K404W, F406L, 1507W, and F512L; N289H, V292F, T295V, K404W, F406L, and F512L; N289H, 1291V, V292F, G293N, T295V, K404W, F406L, and F512L; V292F, G293Q, K404W, and F406L; N289H, V292F, G293I, T295V, F406L, 1407G, and F512L; V292F, G293Q, K404W, F406L, 1407G, and F512L; N289H, I291V, V292F, G2931, T295V, F406L, 1407G, and F512L; V292F, T295V, K404W, F406L, 1507W, and F512L; N289H, V292M, G293I, F406L, F512L, and M480I; N289H, 1291V, V292F, G293Q, T295V, K404W, and F406L; V292F, G293Q, T295V, F406L, and F512L; V292F, T295V, K404W, F406L, and F512L; 1291V, V292F, G293I, K404W, F406L, and I507W; V292F, G293I, T295V, F406L, 1407G, and F512L; N289H, V292F, G293I, T295V, F406L, 1507W, and F512L; N289H, 1291V, V292F, G293I, T295V, K404W, F406L, and F512L; N289H, V292F, G293Q, T295V, F406L, 1407G, and F512L; N289H, 1291V, V292F, G293N, T295V, K404W, and F406L; N289H, V292F, G293Q, F406L, 1507W, and F512L; 1291V, V292F, G293N, T295V, K404W, F406L, 1507W, and F512L; N289H, V292F, G293N, K404W, F406L, 1507W, and F512L; N289H, V292F, G293I, T295V, F406L, 1407G, 1507W, and F512L; N289H, 1291V, V292F, G293N, T295V, F406L, and F512L; 1291V, V292F, G293Q, K404W, F406L, 1407G, and F512L; I291V, V292F, G293Q, T295V, K404W, F406L, 1407G, and F512L; N289H, V292F, T295V, F406L, and F512L; N289H, V292F, K404W, F406L, and F512L; V292F, G293N, T295V, F406L, and F512L; N289H, 1291V, V292F, G293N, T295V, F406L, 1407G, 1507W, and F512L; N289H, 1291V, V292F, G293I, T295V, F406L, and F512L; N289H, 1291V, V292F, G293Q, T295V, F406L, and I507W; N289H, V292F, G293I, F406L, 1407G, 1507W, and F512L; N289H, V292F, G293Q, T295V, K404W, F406L, and I507W; 1291V, V292F, G293Q, T295V, F406L, 1507W, and F512L; S122N, N289H, 1291V, V292F, G293Q, T295V, K404W, F406L, and F512L; P124S, N289H, 1291V, V292F, G293Q, T295V, K404W, F406L, and F512L; T153S, N289H, 1291V, V292F, G293Q, T295V, D346E, K404W, F406L, and F512L; P124S, N289H, 1291V, V292F, G293Q, T295V, K404W, and F406L; R290K, V292F, T295V, K404W, F406L, and F512L; N289H, I291V, V292F, G293Q, T295V, K404W, F406L, Y442L, and F512L; N289H, V292F, G293N, T295V, K404W, F406L, Y442L, and F512L; T721, N289H, 1291V, V292F, G293Q, T295V, K404W, F406L, T494E, and F512L; T72I, N289H, V292F, T295V, K404W, F406L, T494E, and F512L; T721, N289H, V292F, G2931, T295V, F406L, 1407G, Y442L, 1507W, and F512L; P124S, V292F, T295V, K404W, F406L, 1507W, and F512L; N289H, V292F, T295V, K404W, F406L, Y442L, and F512L; N289H, F406L, F512L, and T295V; N289H, F406L, F512L, T295V, and G274S; N289H, F406L, F512L, T295V, and V201I; or N289H, F406L, F512L, T295V, and I398C.

In some embodiments, the sesquiterpene synthase comprises a sequence that is at least 90% identical to any one of SEQ ID NOs: 3-39 or 77-92.

In some embodiments, the sesquiterpene synthase that comprises the amino acid substitutions N289H, V292F, G293N, T295V, K404W, F406L, and F512L relative to SEQ ID NO: 1 comprises the sequence of SEQ ID NO: 3; the sesquiterpene synthase that comprises the amino acid substitutions N289H, 1291V, V292F, G293Q, T295V, K404W, F406L, and F512L relative to SEQ ID NO: 1 comprises the sequence of SEQ ID NO: 4; the sesquiterpene synthase that comprises the amino acid substitutions N289H, F406L, F512L, D191N, and A205E relative to SEQ ID NO: 1 comprises the sequence of SEQ ID NO: 5; the sesquiterpene synthase that comprises the amino acid substitutions 1291V, V292F, G293Q, T295V, K404W, F406L, 1507W, and F512L relative to SEQ ID NO: 1 comprises the sequence of SEQ ID NO: 6; the sesquiterpene synthase that comprises the amino acid substitutions N289H, V292F, T295V, K404W, F406L, and F512L relative to SEQ ID NO: 1 comprises the sequence of SEQ ID NO: 7; the sesquiterpene synthase that comprises the amino acid substitutions N289H, 1291V, V292F, G293N, T295V, K404W, F406L, and F512L relative to SEQ ID NO: 1 comprises the sequence of SEQ ID NO: 8; the sesquiterpene synthase that comprises the amino acid substitutions V292F, G293Q, K404W, and F406L relative to SEQ ID NO: 1 comprises the sequence of SEQ ID NO: 9; the sesquiterpene synthase that comprises the amino acid substitutions N289H, V292F, G293I, T295V, F406L, 1407G, and F512L relative to SEQ ID NO: 1 comprises the sequence of SEQ ID NO: 10; the sesquiterpene synthase that comprises the amino acid substitutions V292F, G293Q, K404W, F406L, 1407G, and F512L relative to SEQ ID NO: 1 comprises the sequence of SEQ ID NO: 11; the sesquiterpene synthase that comprises the amino acid substitutions N289H, 1291V, V292F, G2931, T295V, F406L, 1407G, and F512L relative to SEQ ID NO: 1 comprises the sequence of SEQ ID NO: 12; the sesquiterpene synthase that comprises the amino acid substitutions V292F, T295V, K404W, F406L, 1507W, and F512L relative to SEQ ID NO: 1 comprises the sequence of SEQ ID NO: 13; the sesquiterpene synthase that comprises the amino acid substitutions N289H, V292M, G293I, F406L, F512L, and M480I relative to SEQ ID NO: 1 comprises the sequence of SEQ ID NO: 14; the sesquiterpene synthase that comprises the amino acid substitutions N289H, 1291V, V292F, G293Q, T295V, K404W, and F406L relative to SEQ ID NO: 1 comprises the sequence of SEQ ID NO: 15; the sesquiterpene synthase that comprises the amino acid substitutions V292F, G293Q, T295V, F406L, and F512L relative to SEQ ID NO: 1 comprises the sequence of SEQ ID NO: 16; the sesquiterpene synthase that comprises the amino acid substitutions V292F, T295V, K404W, F406L, and F512L relative to SEQ ID NO: 1 comprises the sequence of SEQ ID NO: 17; the sesquiterpene synthase that comprises the amino acid substitutions I291V, V292F, G293I, K404W, F406L, and I507W relative to SEQ ID NO: 1 comprises the sequence of SEQ ID NO: 18; the sesquiterpene synthase that comprises the amino acid substitutions V292F, G293I, T295V, F406L, 1407G, and F512L relative to SEQ ID NO: 1 comprises the sequence of SEQ ID NO: 19; the sesquiterpene synthase that comprises the amino acid substitutions N289H, V292F, G2931, T295V, F406L, 1507W, and F512L relative to SEQ ID NO: 1 comprises the sequence of SEQ ID NO: 20; the sesquiterpene synthase that comprises the amino acid substitutions N289H, 1291V, V292F, G2931, T295V, K404W, F406L, and F512L relative to SEQ ID NO: 1 comprises the sequence of SEQ ID NO: 21; the sesquiterpene synthase that comprises the amino acid substitutions N289H, V292F, G293Q, T295V, F406L, 1407G, and F512L relative to SEQ ID NO: 1 comprises the sequence of SEQ ID NO: 22; the sesquiterpene synthase that comprises the amino acid substitutions N289H, 1291V, V292F, G293N, T295V, K404W, and F406L relative to SEQ ID NO: 1 comprises the sequence of SEQ ID NO: 23; the sesquiterpene synthase that comprises the amino acid substitutions N289H, V292F, G293Q, F406L, 1507W, and F512L relative to SEQ ID NO: 1 comprises the sequence of SEQ ID NO: 24; the sesquiterpene synthase that comprises the amino acid substitutions 1291V, V292F, G293N, T295V, K404W, F406L, 1507W, and F512L relative to SEQ ID NO: 1 comprises the sequence of SEQ ID NO: 25; the sesquiterpene synthase that comprises the amino acid substitutions N289H, V292F, G293N, K404W, F406L, 1507W, and F512L relative to SEQ ID NO: 1 comprises the sequence of SEQ ID NO: 26; the sesquiterpene synthase that comprises the amino acid substitutions N289H, V292F, G293I, T295V, F406L, 1407G, 1507W, and F512L relative to SEQ ID NO: 1 comprises the sequence of SEQ ID NO: 27; the sesquiterpene synthase that comprises the amino acid substitutions N289H, 1291V, V292F, G293N, T295V, F406L, and F512L relative to SEQ ID NO: 1 comprises the sequence of SEQ ID NO: 28; the sesquiterpene synthase that comprises the amino acid substitutions I291V, V292F, G293Q, K404W, F406L, 1407G, and F512L relative to SEQ ID NO: 1 comprises the sequence of SEQ ID NO: 29; the sesquiterpene synthase that comprises the amino acid substitutions I291V, V292F, G293Q, T295V, K404W, F406L, 1407G, and F512L relative to SEQ ID NO: 1 comprises the sequence of SEQ ID NO: 30; the sesquiterpene synthase that comprises the amino acid substitutions N289H, V292F, T295V, F406L, and F512L relative to SEQ ID NO: 1 comprises the sequence of SEQ ID NO: 31; the sesquiterpene synthase that comprises the amino acid substitutions N289H, V292F, K404W, F406L, and F512L relative to SEQ ID NO: 1 comprises the sequence of SEQ ID NO: 32; the sesquiterpene synthase that comprises the amino acid substitutions V292F, G293N, T295V, F406L, and F512L relative to SEQ ID NO: 1 comprises the sequence of SEQ ID NO: 33; the sesquiterpene synthase that comprises the amino acid substitutions N289H, 1291V, V292F, G293N, T295V, F406L, 1407G, 1507W, and F512L relative to SEQ ID NO: 1 comprises the sequence of SEQ ID NO: 34; the sesquiterpene synthase that comprises the amino acid substitutions N289H, 1291V, V292F, G2931, T295V, F406L, and F512L relative to SEQ ID NO: 1 comprises the sequence of SEQ ID NO: 35; the sesquiterpene synthase that comprises the amino acid substitutions N289H, 1291V, V292F, G293Q, T295V, F406L, and I507W relative to SEQ ID NO: 1 comprises the sequence of SEQ ID NO: 36; the sesquiterpene synthase that comprises the amino acid substitutions N289H, V292F, G2931, F406L, 1407G, 1507W, and F512L relative to SEQ ID NO: 1 comprises the sequence of SEQ ID NO: 37; the sesquiterpene synthase that comprises the amino acid substitutions N289H, V292F, G293Q, T295V, K404W, F406L, and I507W relative to SEQ ID NO: 1 comprises the sequence of SEQ ID NO: 38; the sesquiterpene synthase that comprises the amino acid substitutions I291V, V292F, G293Q, T295V, F406L, 1507W, and F512L relative to SEQ ID NO: 1 comprises the sequence of SEQ ID NO: 39; the sesquiterpene synthase that comprises the amino acid substitutions S122N, N289H, 1291V, V292F, G293Q, T295V, K404W, F406L, and F512L relative to SEQ ID NO: 1 comprises the sequence of SEQ ID NO: 84; the sesquiterpene synthase that comprises the amino acid substitutions P124S, N289H, 1291V, V292F, G293Q, T295V, K404W, F406L, and F512L relative to SEQ ID NO: 1 comprises the sequence of SEQ ID NO: 80; the sesquiterpene synthase that comprises the amino acid substitutions T153S, N289H, 1291V, V292F, G293Q, T295V, D346E, K404W, F406L, and F512L relative to SEQ ID NO: 1 comprises the sequence of SEQ ID NO: 82; the sesquiterpene synthase that comprises the amino acid substitutions P124S, N289H, 1291V, V292F, G293Q, T295V, K404W, and F406L relative to SEQ ID NO: 1 comprises the sequence of SEQ ID NO: 81; the sesquiterpene synthase that comprises the amino acid substitutions R290K, V292F, T295V, K404W, F406L, and F512L relative to SEQ ID NO: 1 comprises the sequence of SEQ ID NO: 83; the sesquiterpene synthase that comprises the amino acid substitutions N289H, 1291V, V292F, G293Q, T295V, K404W, F406L, Y442L, and F512L relative to SEQ ID NO: 1 comprises the sequence of SEQ ID NO: 78; the sesquiterpene synthase that comprises the amino acid substitutions N289H, V292F, G293N, T295V, K404W, F406L, Y442L, and F512L relative to SEQ ID NO: 1 comprises the sequence of SEQ ID NO: 77; the sesquiterpene synthase that comprises the amino acid substitutions T721, N289H, 1291V, V292F, G293Q, T295V, K404W, F406L, T494E, and F512L relative to SEQ ID NO: 1 comprises the sequence of SEQ ID NO: 79; the sesquiterpene synthase that comprises the amino acid substitutions T721, N289H, V292F, T295V, K404W, F406L, T494E, and F512L relative to SEQ ID NO: 1 comprises the sequence of SEQ ID NO: 88; the sesquiterpene synthase that comprises the amino acid substitutions T72I, N289H, V292F, G293I, T295V, F406L, 1407G, Y442L, 1507W, and F512L relative to SEQ ID NO: 1 comprises the sequence of SEQ ID NO: 86; the sesquiterpene synthase that comprises the amino acid substitutions P124S, V292F, T295V, K404W, F406L, 1507W, and F512L relative to SEQ ID NO: 1 comprises the sequence of SEQ ID NO: 85; the sesquiterpene synthase that comprises the amino acid substitutions N289H, V292F, T295V, K404W, F406L, Y442L, and F512L relative to SEQ ID NO: 1 comprises the sequence of SEQ ID NO: 87; the sesquiterpene synthase that comprises the amino acid substitutions N289H, F406L, F512L, and T295V relative to SEQ ID NO: 1 comprises the sequence of SEQ ID NO: 89; the sesquiterpene synthase that comprises the amino acid substitutions N289H, F406L, F512L, T295V, and G274S relative to SEQ ID NO: 1 comprises the sequence of SEQ ID NO: 90; the sesquiterpene synthase that comprises the amino acid substitutions N289H, F406L, F512L, T295V, and V201I relative to SEQ ID NO: 1 comprises the sequence of SEQ ID NO: 91; or the sesquiterpene synthase that comprises the amino acid substitutions N289H, F406L, F512L, T295V, and 1398C relative to SEQ ID NO: 1 comprises the sequence of SEQ ID NO: 92.

In some embodiments, the heterologous nucleic acid comprises a sequence that is at least 90% identical to any one of SEQ ID NOs: 40-76 or 93-109.

In some embodiments, the sesquiterpene synthase further comprises at least one amino acid substitution at a position corresponding to position 294, 296, 297, 403, 444, 515, and/or 525 in SEQ ID NO: 1.

In some embodiments, the sesquiterpene synthase comprises: an L, C, Y, V, A, or S residue at a position corresponding to position 294 in SEQ ID NO: 1; an L, A, proline (P), Y, N, F or R residue at a position corresponding to position 296 in SEQ ID NO: 1; an E, Y, I, lysine (K), M, or H residue at a position corresponding to position 297 in SEQ ID NO: 1; an M, Q, N, S, T, A, E, H, C, or V residue at a position corresponding to position 403 in SEQ ID NO: 1; an A or N residue at a position corresponding to position 444 in SEQ ID NO: 1; an H, A, E, or Q residue at a position corresponding to position 515 in SEQ ID NO: 1; an H, C, L, or N residue at a position corresponding to position 525 in SEQ ID NO: 1; or any combination thereof.

In some embodiments, the host cell is capable of producing more alpha-guaiene than a host cell that expresses a sesquiterpene synthase that comprises the sequence of SEQ ID NO: 1. In some embodiments, the host cell produces more alpha-guaiene than delta-guaiene.

In some embodiments, the sesquiterpene synthase comprises: (a) one or more amino acid substitutions in a first region of the active site relative to SEQ ID NO: 1, wherein the one or more amino acid substitutions in the first region are at positions that correspond to positions 295, 291, 406, 512, and/or 519 of SEQ ID NO: 1, and the one or more amino acid substitutions in the first region each comprises a residue with a smaller side chain than that of the amino acid at positions 295, 291, 406, 512, and/or 519 in SEQ ID NO: 1; (b) an amino acid substitution in a second region of the active site relative to SEQ ID NO: 1, wherein the amino acid substitution in the second region corresponds to position 292 of SEQ ID NO: 1, and wherein the amino acid substitution in the second region comprises a larger side chain than that of the amino acid at position 292 in SEQ ID NO: 1; or (c) any combination thereof. In some embodiments, the side chain in (a) is hydrophobic.

In some embodiments, the sesquiterpene synthase comprises: a V residue at a position corresponding to position 295 in SEQ ID NO: 1; a C residue at a position corresponding to position 291 in SEQ ID NO: 1; a L residue at a position corresponding to position 406 in SEQ ID NO: 1; a L residue at a position corresponding to position 512 in SEQ ID NO: 1; a F residue at a position corresponding to position 292 in SEQ ID NO: 1; or any combination thereof.

In some embodiments, the sesquiterpene synthase further comprises one or more amino acid substitutions relative to SEQ ID NO: 1 at one or more positions corresponding to position 294, 296, 297, 403, 444, 515, and/or 525 in SEQ ID NO: 1.

In some embodiments, the sesquiterpene synthase comprises: a L, T, S, D, or M residue at a position corresponding to position 289 in SEQ ID NO: 1; an F, L, T, or C residue at a position corresponding to position 291 in SEQ ID NO: 1; an A, Q, C, Y, H, E, F, T, or W residue at a position corresponding to position 292 in SEQ ID NO: 1; a L, V, T, Y, C, F, W, or M residue at a position corresponding to position 293 in SEQ ID NO: 1; a L, C, Y, V, A, or S residue at a position corresponding to position 294 in SEQ ID NO: 1; an E, D, N, W, G, or I residue at a position corresponding to position 295 in SEQ ID NO: 1; a L, A, P, Y, N, arginine (R), or F residue at a position corresponding to position 296 in SEQ ID NO: 1; an E, Y, I, K, M, or H residue at a position corresponding to position 297 in SEQ ID NO: 1; a M, Q, N, V, C, H, E, A, T, or S residue at a position corresponding to position 403 in SEQ ID NO: 1; a L, T, W, or N residue at a position corresponding to position 406 in SEQ ID NO: 1; an A or N residue at a position corresponding to position 444 in SEQ ID NO: 1; an H, A, E, or Q residue at a position corresponding to position 515 in SEQ ID NO: 1; an H, C, L, or N residue at a position corresponding to position 525 in SEQ ID NO: 1; or any combination thereof.

In some embodiments, the sesquiterpene synthase does not comprise: a Q, C, V, F, A, I, H, G, W, or Y residue at a position corresponding to position 289 in SEQ ID NO: 1; a V, M, or A residue at a position corresponding to position 291 in SEQ ID NO: 1; an I, M, S, L, G, or N residue at a position corresponding to position 292 in SEQ ID NO: 1; an I, Q, S, N, or E residue at a position corresponding to position 293 in SEQ ID NO: 1; a L, V, A, or S residue at a position corresponding to position 295 in SEQ ID NO: 1; a K or Q residue at a position corresponding to position 296 in SEQ ID NO: 1; a T or A residue at a position corresponding to position 297 in SEQ ID NO: 1; a P, F, I, L, G, or D residue at a position corresponding to position 403 in SEQ ID NO: 1; a G, S, A, I, Y, M, H, V, Q, or C residue at a position corresponding to position 406 in SEQ ID NO: 1; an H residue at a position corresponding to position 444 in SEQ ID NO: 1; a M, C, F, G, N, S, or I residue at a position corresponding to position 515 in SEQ ID NO: 1; or any combination thereof.

Further aspects of the disclosure relate to non-naturally occurring sesquiterpene synthases, wherein the sequence of the sesquiterpene synthase comprises one or more amino acid substitutions relative to SEQ ID NO: 1, wherein at least one of the amino acid substitutions is at a position corresponding to position 122, 274, 275, 293, 295, 301, 368, 398, 404, 407, 507, 515, 525, and/or 533 in SEQ ID NO: 1. In some embodiments, the sequence of the sesquiterpene synthase further comprises at least one amino acid substitution at a position corresponding to position 72, 124, 153, 191, 201, 205, 289, 290, 291, 292, 346, 406, 442, 480, 494, 509, 512, and/or 526 in SEQ ID NO: 1.

Further aspects of the disclosure relate to non-naturally occurring sesquiterpene synthases, wherein the sequence of the sesquiterpene synthases comprises two or more amino acid substitutions relative to SEQ ID NO: 1, wherein: at least one amino acid substitution is at a position corresponding to position 122, 274, 275, 293, 295, 301, 368, 398, 404, 407, 507, 515, 525, and/or 533 in SEQ ID NO: 1; and at least one amino acid substitution is at a position corresponding to position 72, 124, 153, 191, 201, 205, 289, 290, 291, 292, 346, 406, 442, 480, 494, 509, 512, and/or 526 in SEQ ID NO: 1.

Further aspects of the disclosure relate to non-naturally occurring sesquiterpene synthases, wherein the sesquiterpene synthase comprises an amino acid sequence that is at least 90% identical to the sequence of SEQ ID NO: 1, wherein the sesquiterpene synthase comprises an amino acid sequence having two or more amino acid substitutions relative to SEQ ID NO: 1, and wherein the two or more amino acid substitutions are at positions corresponding to position 72, 122, 124, 153, 191, 201, 205, 274, 275, 289, 290, 291, 292, 293, 295, 301, 346, 368, 398, 404, 406, 407, 442, 480, 494, 507, 509, 512, 526 and/or 533 in SEQ ID NO: 1, wherein the sesquiterpene synthase is capable of producing sesquiterpene products, and wherein at least 50% of sesquiterpene products produced by the sesquiterpene synthase is alpha-guaiene.

In some embodiments, the amino acid sequence of the sesquiterpene synthase is at least 90% identical to any one of SEQ ID NOs: 3-39 or 77-92.

Further aspects of the disclosure relate to non-naturally occurring nucleic acids encoding a sesquiterpene synthase, wherein the non-naturally occurring nucleic acid comprises a sequence that is at least 90% identical to SEQ ID NOs: 40-76 or 93-109.

Further aspects of the disclosure relate to methods of producing a sesquiterpene comprising culturing host cells associated with the disclosure in culture medium in the presence of an FPP substrate and optionally isolating or recovering the sesquiterpene from the host cell and/or the culture medium. In some embodiments, the sesquiterpene is alpha-guaiene. In some embodiments, the sesquiterpene is aciphyllene.

In some embodiments, at least 50% of the sesquiterpenes isolated or recovered from the host cell or culture medium is alpha-guaiene. In some embodiments, at least 15% of the sesquiterpenes isolated or recovered from the host cell or culture medium is aciphyllene. In some embodiments, the alpha-guaiene is recovered from the culture medium. In some embodiments, the aciphyllene is recovered from the culture medium. In some embodiments, the method further comprises obtaining a composition comprising alpha-guaiene. In some embodiments, the method further comprises obtaining a composition comprising aciphyllene.

Further aspects of the disclosure relate to sesquiterpenes obtainable from host cells associated with the disclosure and/or from methods associated with the disclosure. In some embodiments, the sesquiterpene is alpha-guaiene. In some embodiments, the sesquiterpene is aciphyllene.

Further aspects of the disclosure relate to culture media comprising sesquiterpenes associated with the disclosure.

Further aspects of the disclosure relate to compositions comprising sesquiterpenes associated with the disclosure. In some embodiments, the sesquiterpene is alpha-guaiene. In some embodiments, the sesquiterpene is aciphyllene.

Further aspects of the disclosure relate to compositions comprising (a) a sesquiterpene, wherein at least 50% of the sesquiterpene is alpha-guaiene, and (b) one or more additional components, wherein the one or more additional components include fermentation medium, cell culture supernatant and/or a hydrophobic overlay. In some embodiments, at least 15% of the sesquiterpene is aciphyllene. In some embodiments, the alpha-guaiene is produced using a microbial host cell. In some embodiments, the alpha-guaiene is produced using an in-vitro or an in-vivo system. In some embodiments, about 50% to about 90% of the sesquiterpene is alpha-guaiene. In some embodiments, the composition further comprises delta-guaiene. In some embodiments, the composition further comprises aciphyllene. In some embodiments, about 50% to about 10% of the sesquiterpene is alpha-guaiene.

In some embodiments, the composition further comprises one or more non-terpene components or one or more additional terpene components. In some embodiments, the one or more non-terpene components include FPP. In some embodiments, the one or more additional terpene components include: delta-guaiene, beta-guaiene, gamma guaiene, germacrene A, aciphyllene, and/or alpha-humulene, as determined by GC.

Further aspects of the disclosure relate to methods of making compositions associated with the disclosure comprising culturing a host cell that comprises a heterologous polynucleotide encoding a sesquiterpene synthase, wherein the sesquiterpene synthase comprises one or more amino acid substitutions relative to SEQ ID NO: 1, wherein at least one of the amino acid substitutions is at a position corresponding to position 122, 274, 275, 293, 295, 301, 368, 398, 404, 407, 507, 515, 525, and/or 533 in SEQ ID NO: 1. In some embodiments, the sequence of the sesquiterpene synthase further comprises at least one amino acid substitution at a position corresponding to position 72, 124, 153, 191, 201, 205, 289, 290, 291, 292, 346, 406, 442, 480, 494, 509, 512, and/or 526 in SEQ ID NO: 1.

Further aspects of the disclosure relate to methods of making compositions associated with the disclosure comprising culturing a host cell that comprises a heterologous polynucleotide encoding a sesquiterpene synthase, wherein the sequence of the sesquiterpene synthase comprises two or more amino acid substitutions relative to SEQ ID NO: 1, wherein: at least one amino acid substitution is at a position corresponding to position 122, 274, 275, 293, 295, 301, 368, 398, 404, 407, 507, 515, 525, and/or 533 in SEQ ID NO: 1; and at least one amino acid substitution is at a position corresponding to position 72, 124, 153, 191, 201, 205, 289, 290, 291, 292, 346, 406, 442, 480, 494, 509, 512, and/or 526 in SEQ ID NO: 1.

Further aspects of the disclosure relate to methods of making compositions associated with the disclosure comprising culturing a host cell that comprises a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, wherein the two or more amino acid substitutions are at positions corresponding to position 72, 122, 124, 153, 191, 201, 205, 274, 275, 289, 290, 291, 292, 293, 295, 301, 346, 368, 398, 404, 406, 407, 442, 480, 494, 507, 509, 512, 526 and/or 533 in SEQ ID NO: 1.

Further aspects of the disclosure relate to host cells that comprise a heterologous polynucleotide encoding a sesquiterpene synthase, wherein the sequence of the sesquiterpene synthase comprises one or more amino acid substitutions relative to SEQ ID NO: 1, and wherein at least one of the amino acid substitutions is at a position corresponding to position 44, 212, 217, 293, 295, 404, 515, and/or 542 in SEQ ID NO: 1. In some embodiments, the sequence of the sesquiterpene synthase further comprises at least one amino acid substitution at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

Further aspects of the disclosure relate to host cells that comprise a heterologous polynucleotide encoding a sesquiterpene synthase, wherein the sequence of the sesquiterpene synthase comprises two or more amino acid substitutions relative to SEQ ID NO: 1, wherein: at least one of the amino acid substitutions is at a position corresponding to position 44, 212, 217, 293, 295, 404, 515, and/or 542 in SEQ ID NO: 1; and at least one of the amino acid substitutions is at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1. In some embodiments, the host cell is capable of producing sesquiterpene products, wherein at least 50% of the total sesquiterpene products produced by the host cell is alpha-guaiene. In some embodiments, at least 15% of the total sesquiterpene products produced by the host cell is aciphyllene.

Further aspects of the disclosure relate to host cells that comprise a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, wherein the two or more amino acid substitutions are at positions corresponding to position 23, 44, 72, 86, 111, 118, 134, 147, 188, 201, 212, 217, 224, 252, 255, 289, 290, 291, 292, 293, 295, 346, 381, 390, 404, 406, 419, 433, 442, 443, 444, 448, 458, 467, 494, 499, 512, 515, 516, 519, 542 in SEQ ID NO: 1, wherein the host cell is capable of producing sesquiterpene products, and wherein at least 50% of the total sesquiterpene products produced by the host cell is alpha-guaiene. In some embodiments, the sesquiterpene synthase comprises 4, 5, 6, 7, 8, 9, 10, 11, 12, 13, 14, 15, 16, 17, 18, 19, 20, 21, 22, 23, 24, 25, 26, 27, or more than 27 amino acid substitutions relative to SEQ ID NO: 1.

In some embodiments, the sesquiterpene synthase comprises: an aspartate (D) residue at a position corresponding to position 23 in SEQ ID NO: 1; a valine (V) residue at a position corresponding to position 44 in SEQ ID NO: 1; an isoleucine (I) residue at a position corresponding to position 72 in SEQ ID NO: 1; a glutamate (E) residue at a position corresponding to position 86 in SEQ ID NO: 1; a leucine (L) residue at a position corresponding to position 111 in SEQ ID NO: 1; a glutamine (Q) residue at a position corresponding to position 118 in SEQ ID NO: 1; a D residue at a position corresponding to position 134 in SEQ ID NO: 1; a V residue at a position corresponding to position 147 in SEQ ID NO: 1; an L residue at a position corresponding to position 147 in SEQ ID NO: 1; a Q residue at a position corresponding to position 188 in SEQ ID NO: 1; a serine(S) residue at a position corresponding to position 201 in SEQ ID NO: 1; a phenylalanine (F) residue at a position corresponding to position 212 in SEQ ID NO: 1; an E residue at a position corresponding to position 217 in SEQ ID NO: 1; an L residue at a position corresponding to position 224 in SEQ ID NO: 1; an L residue at a position corresponding to position 252 in SEQ ID NO: 1; an alanine (A) residue at a position corresponding to position 255 in SEQ ID NO: 1; a lysine (K) residue at a position corresponding to position 289 in SEQ ID NO: 1; a histidine (H) residue at a position corresponding to position 290 in SEQ ID NO: 1; a V residue at a position corresponding to position 291 in SEQ ID NO: 1; a cysteine (C) residue at a position corresponding to position 292 in SEQ ID NO: 1; an F residue at a position corresponding to position 292 in SEQ ID NO: 1; an asparagine (N) residue at a position corresponding to position 293 in SEQ ID NO: 1; a Q residue at a position corresponding to position 293 in SEQ ID NO: 1; an I residue at a position corresponding to position 295 in SEQ ID NO: 1; a V residue at a position corresponding to position 295 in SEQ ID NO: 1; an E residue at a position corresponding to position 346 in SEQ ID NO: 1; a tryptophan (W) residue at a position corresponding to position 381 in SEQ ID NO: 1; an F residue at a position corresponding to position 390 in SEQ ID NO: 1; a W residue at a position corresponding to position 404 in SEQ ID NO: 1; an L residue at a position corresponding to position 406 in SEQ ID NO: 1; an S residue at a position corresponding to position 419 in SEQ ID NO: 1; an I residue at a position corresponding to position 433 in SEQ ID NO: 1; an L residue at a position corresponding to position 433 in SEQ ID NO: 1; an L residue at a position corresponding to position 442 in SEQ ID NO: 1; a W residue at a position corresponding to position 443 in SEQ ID NO: 1; a threonine (T) residue at a position corresponding to position 443 in SEQ ID NO: 1; an N residue at a position corresponding to position 444 in SEQ ID NO: 1; a G residue at a position corresponding to position 448 in SEQ ID NO: 1; an L residue at a position corresponding to position 458 in SEQ ID NO: 1; a V residue at a position corresponding to position 458 in SEQ ID NO: 1; a K residue at a position corresponding to position 467 in SEQ ID NO: 1; an E residue at a position corresponding to position 494 in SEQ ID NO: 1; an N residue at a position corresponding to position 499 in SEQ ID NO: 1; an L residue at a position corresponding to position 512 in SEQ ID NO: 1; a methionine (M) residue at a position corresponding to position 515 in SEQ ID NO: 1; an A residue at a position corresponding to position 516 in SEQ ID NO: 1; a V residue at a position corresponding to position 516 in SEQ ID NO: 1; an L residue at a position corresponding to position 519 in SEQ ID NO: 1; a V residue at a position corresponding to position 542 in SEQ ID NO: 1; or any combination thereof.

In some embodiments, the sesquiterpene synthase comprises the following amino acid substitutions relative to SEQ ID NO: 1: T721, N289H, R290K, 1291V, V292F, G293Q, T295V, K404W, F406L, Y442L, T494E, and F512L; L44V, T72I, N289H, 1291V, V292F, G293Q, T295V, K404W, F406L, Y442L, and F512L; L44V, T72I, T86E, G118Q, T134D, W147L, Q212F, Q217E, S224L, F252L, P255A, N289H, R290K, 1291V, V292F, G293Q, T295V, D346E, Y390F, K404W, F406L, L419S, Y442L, T458V, R467K, F512L, and R542V; T23D, L44V, T721, N289H, 1291V, V292F, G293Q, T295V, K404W, F406L, Y442L, and F512L; V201S, N289H, 1291V, V292F, G293N, T295V, Y381W, K404W, F406L, T494E, and F512L; V201S, V292C, T295I, and D444N; V201S, V292C, T295I, D444N, and L516V; L44V, T72I, V201S, V292C, T295I, D444N, and L516V; L44V, T72I, V201S, V292C, T295I, D444N, and L516A; L44V, T72I, V201S, V292C, T2951, 1443W, and D444N; L44V, T72I, V201S, V292C, T295I, D444N, and M519L; W147V, C188Q, V201S, S224L, V292C, T2951, and D444N; L44V, T72I, V111L, V201S, S224L, V292C, T2951, 1443T, D444N, T448G, and M519L; L44V, T72I, V11IL, V201S, V292C, T295I, V4331, 1443T, D444N, T448G, D499N, and L516V; L44V, T72I, V201S, V292C, T295I, D444N, L516V, and M519L; L44V, T72I, V201S, V292C, T295I, D444N, V515M, and L516V; V11IL, V201S, S224L, V292C, T2951, 1443T, D444N, and L516V; V201S, V292C, T2951, D444N, and L516V; V201S, V292C, T2951, D444N, T458L, and L516A; V201S, V292C, T2951, 1443T, D444N, and L516V; V201S, V292C, T295I, V433I, D444N, and L516V; or V201S, V292C, T295I, V433L, D444N, and L516V.

In some embodiments, the sesquiterpene synthase comprises a sequence that is at least 90% identical to any one of SEQ ID NOs: 110-131.

In some embodiments, the sesquiterpene synthase that comprises the amino acid substitutions T721, N289H, R290K, 1291V, V292F, G293Q, T295V, K404W, F406L, Y442L, T494E, and F512L relative to SEQ ID NO: 1 comprises the sequence of SEQ ID NO: 110; the sesquiterpene synthase that comprises the amino acid substitutions L44V, T72I, N289H, 1291V, V292F, G293Q, T295V, K404W, F406L, Y442L, and F512L relative to SEQ ID NO: 1 comprises the sequence of SEQ ID NO: 111; the sesquiterpene synthase that comprises the amino acid substitutions L44V, T72I, T86E, G118Q, T134D, W147L, Q212F, Q217E, S224L, F252L, P255A, N289H, R290K, 1291V, V292F, G293Q, T295V, D346E, Y390F, K404W, F406L, L419S, Y442L, T458V, R467K, F512L, and R542V relative to SEQ ID NO: 1 comprises the sequence of SEQ ID NO: 112; the sesquiterpene synthase that comprises the amino acid substitutions T23D, L44V, T721, N289H, 1291V, V292F, G293Q, T295V, K404W, F406L, Y442L, and F512L relative to SEQ ID NO: 1 comprises the sequence of SEQ ID NO: 113; the sesquiterpene synthase that comprises the amino acid substitutions V201S, N289H, 1291V, V292F, G293N, T295V, Y381W, K404W, F406L, T494E, and F512L relative to SEQ ID NO: 1 comprises the sequence of SEQ ID NO: 114; the sesquiterpene synthase that comprises the amino acid substitutions V201S, V292C, T2951, and D444N relative to SEQ ID NO: 1 comprises the sequence of SEQ ID NO: 115; the sesquiterpene synthase that comprises the amino acid substitutions V201S, V292C, T2951, D444N, and L516V relative to SEQ ID NO: 1 comprises the sequence of SEQ ID NO: 116; the sesquiterpene synthase that comprises the amino acid substitutions L44V, T72I, V201S, V292C, T2951, D444N, and L516V relative to SEQ ID NO: 1 comprises the sequence of SEQ ID NO: 117; the sesquiterpene synthase that comprises the amino acid substitutions L44V, T72I, V201S, V292C, T2951, D444N, and L516A relative to SEQ ID NO: 1 comprises the sequence of SEQ ID NO: 118; the sesquiterpene synthase that comprises the amino acid substitutions L44V, T72I, V201S, V292C, T2951, 1443W, and D444N relative to SEQ ID NO: 1 comprises the sequence of SEQ ID NO: 119; the sesquiterpene synthase that comprises the amino acid substitutions L44V, T72I, V201S, V292C, T295I, D444N, and M519L relative to SEQ ID NO: 1 comprises the sequence of SEQ ID NO: 120; the sesquiterpene synthase that comprises the amino acid substitutions W147V, C188Q, V201S, S224L, V292C, T295I, and D444N relative to SEQ ID NO: 1 comprises the sequence of SEQ ID NO: 121; the sesquiterpene synthase that comprises the amino acid substitutions L44V, T721, V11IL, V201S, S224L, V292C, T2951, 1443T, D444N, T448G, and M519L relative to SEQ ID NO: 1 comprises the sequence of SEQ ID NO: 122; the sesquiterpene synthase that comprises the amino acid substitutions L44V, T72I, V111L, V201S, V292C, T295I, V433I, 1443T, D444N, T448G, D499N, and L516V relative to SEQ ID NO: 1 comprises the sequence of SEQ ID NO: 123; the sesquiterpene synthase that comprises the amino acid substitutions L44V, T72I, V201S, V292C, T295I, D444N, L516V, and M519L relative to SEQ ID NO: 1 comprises the sequence of SEQ ID NO: 124; the sesquiterpene synthase that comprises the amino acid substitutions L44V, T72I, V201S, V292C, T295I, D444N, V515M, and L516V relative to SEQ ID NO: 1 comprises the sequence of SEQ ID NO: 125; the sesquiterpene synthase that comprises the amino acid substitutions V111L, V201S, S224L, V292C, T2951, 1443T, D444N, and L516V relative to SEQ ID NO: 1 comprises the sequence of SEQ ID NO: 126; the sesquiterpene synthase that comprises the amino acid substitutions V201S, V292C, T2951, D444N, and L516V relative to SEQ ID NO: 1 comprises the sequence of SEQ ID NO: 127; the sesquiterpene synthase that comprises the amino acid substitutions V201S, V292C, T2951, D444N, T458L, and L516A relative to SEQ ID NO: 1 comprises the sequence of SEQ ID NO: 128; the sesquiterpene synthase that comprises the amino acid substitutions V201S, V292C, T2951, 1443T, D444N, and L516V relative to SEQ ID NO: 1 comprises the sequence of SEQ ID NO: 129; the sesquiterpene synthase that comprises the amino acid substitutions V201S, V292C, T295I, V433I, D444N, and L516V relative to SEQ ID NO: 1 comprises the sequence of SEQ ID NO: 130; or the sesquiterpene synthase that comprises the amino acid substitutions V201S, V292C, T295I, V433L, D444N, and L516V relative to SEQ ID NO: 1 comprises the sequence of SEQ ID NO: 131.

In some embodiments, the heterologous nucleic acid comprises a sequence that is at least 90% identical to any one of SEQ ID NOs: 133-154.

In some embodiments, the host cell is capable of producing more alpha-guaiene than a host cell that expresses a sesquiterpene synthase that comprises the sequence of SEQ ID NO: 1. In some embodiments, the host cell produces more alpha-guaiene than delta-guaiene. In some embodiments, the host cell produces more aciphyllene than delta-guaiene.

Further aspects of the disclosure relate to non-naturally occurring sesquiterpene synthases, wherein the sequence of the sesquiterpene synthase comprises one or more amino acid substitutions relative to SEQ ID NO: 1, wherein at least one of the amino acid substitutions is at a position corresponding to position 44, 212, 217, 293, 295, 404, 515, and/or 542 in SEQ ID NO: 1. In some embodiments, the sequence of the sesquiterpene synthase further comprises at least one amino acid substitution at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

Further aspects of the disclosure relate to non-naturally occurring sesquiterpene synthases, wherein the sequence of the sesquiterpene synthase comprises two or more amino acid substitutions relative to SEQ ID NO: 1, wherein: at least one amino acid substitution is at a position corresponding to position 44, 212, 217, 293, 295, 404, 515, and/or 542 in SEQ ID NO: 1; and at least one amino acid substitution is at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

Further aspects of the disclosure relate to non-naturally occurring sesquiterpene synthases, wherein the sesquiterpene synthase comprises an amino acid sequence that is at least 90% identical to the sequence of SEQ ID NO: 1, wherein the sesquiterpene synthase comprises an amino acid sequence having two or more amino acid substitutions relative to SEQ ID NO: 1, and wherein the two or more amino acid substitutions are at positions corresponding to position 23, 44, 72, 86, 111, 118, 134, 147, 188, 201, 212, 217, 224, 252, 255, 289, 290, 291, 292, 293, 295, 346, 381, 390, 404, 406, 419, 433, 442, 443, 444, 448, 458, 467, 494, 499, 512, 515, 516, 519, or 542 in SEQ ID NO: 1, wherein the sesquiterpene synthase is capable of producing sesquiterpene products, and wherein at least 50% of sesquiterpene products produced by the sesquiterpene synthase is alpha-guaiene.

In some embodiments, the amino acid sequence of the sesquiterpene synthase is at least 90% identical to any one of SEQ ID NOs: 110-131.

Further aspects of the disclosure relate to non-naturally occurring nucleic acids encoding sesquiterpene synthases, wherein the non-naturally occurring nucleic acid comprises a sequence that is at least 90% identical to any one of SEQ ID NOs: 133-154.

Further aspects of the disclosure relate to methods of producing sesquiterpenes comprising culturing a host cell associated with the disclosure in culture medium in the presence of an FPP substrate and optionally isolating or recovering the sesquiterpene from the host cell and/or the culture medium. In some embodiments, the sesquiterpene is alpha-guaiene. In some embodiments, the sesquiterpene is aciphyllene. In some embodiments, 50% of the sesquiterpenes isolated or recovered from the host cell or culture medium is alpha-guaiene. In some embodiments, at least 15% of the sesquiterpenes isolated or recovered from the host cell or culture medium is aciphyllene. In some embodiments, the alpha-guaiene is recovered from the culture medium. In some embodiments, the aciphyllene is recovered from the culture medium. In some embodiments, the method further comprises obtaining a composition comprising alpha-guaiene. In some embodiments, the method further comprises obtaining a composition comprising aciphyllene.

Further aspects of the disclosure relate to sesquiterpenes obtainable from a host cell associated with the disclosure or from a method associated with this disclosure. In some embodiments, the sesquiterpene is alpha-guaiene. In some embodiments, the sesquiterpene is aciphyllene.

Further aspects of the disclosure relate to culture media comprising a sesquiterpene associated with the disclosure.

Further aspects of the disclosure relate to compositions comprising a sesquiterpene associated with the disclosure. In some embodiments, the sesquiterpene is alpha-guaiene. In some embodiments, the sesquiterpene is aciphyllene.

Further aspects of the disclosure relate to compositions comprising (a) a sesquiterpene, wherein at least 50% of the sesquiterpene is alpha-guaiene, and (b) one or more additional components, wherein the one or more additional components include fermentation medium, cell culture supernatant and/or a hydrophobic overlay. In some embodiments, at least 15% of the sesquiterpene is aciphyllene. In some embodiments, the alpha-guaiene is produced using a microbial host cell. In some embodiments, the alpha-guaiene is produced using an in-vitro or an in-vivo system. In some embodiments, about 50% to about 90% of the sesquiterpene is alpha-guaiene. In some embodiments, the composition further comprises delta-guaiene. In some embodiments, the composition further comprises aciphyllene. In some embodiments, about 50% to about 10% of the sesquiterpene is alpha-guaiene. In some embodiments, at least 15% of the sesquiterpene is aciphyllene.

In some embodiments, the composition further comprises one or more non-terpene components or one or more additional terpene components. In some embodiments, the one or more non-terpene components include FPP. In some embodiments, the one or more additional terpene components include: delta-guaiene, beta-guaiene, gamma guaiene, germacrene A, aciphyllene, and/or alpha-humulene, as determined by GC.

Further aspects of the disclosure relate to methods of making compositions associated with the disclosure comprising culturing a host cell that comprises a heterologous polynucleotide encoding a sesquiterpene synthase, wherein the sesquiterpene synthase comprises one or more amino acid substitutions relative to SEQ ID NO: 1, wherein at least one of the amino acid substitutions is at a position corresponding to position 44, 212, 217, 293, 295, 404, 448, 515, and/or 542 in SEQ ID NO: 1. In some embodiments, the sequence of the sesquiterpene synthase further comprises at least one amino acid substitution at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

Further aspects of the disclosure relate to methods of making compositions associated with the disclosure comprising culturing a host cell that comprises a heterologous polynucleotide encoding a sesquiterpene synthase, wherein the sequence of the sesquiterpene synthase comprises two or more amino acid substitutions relative to SEQ ID NO: 1, wherein: at least one amino acid substitution is at a position corresponding to position 44, 212, 217, 293, 295, 404, 515, and/or 542 in SEQ ID NO: 1; and at least one amino acid substitution is at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

Further aspects of the disclosure relate to methods of making compositions associated with the disclosure comprising culturing a host cell that comprises a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, and wherein the two or more amino acid substitutions are at positions corresponding to position 23, 44, 72, 86, 111, 118, 134, 147, 188, 201, 212, 217, 224, 252, 255, 289, 290, 291, 292, 293, 295, 346, 381, 390, 404, 406, 419, 433, 442, 443, 444, 448, 458, 467, 494, 499, 512, 515, 516, 519, or 542 in SEQ ID NO: 1.

Each of the limitations of the compositions and methods described in this disclosure may encompass various described embodiments. It is, therefore, anticipated that each of the limitations of the invention involving any one element or combinations of elements can be included in each aspect of the invention. This present disclosure is not limited in its application to the details of construction and the arrangement of components set forth in the following description or illustrated in the drawings.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1 depicts the sesquiterpene profile produced by Delta-guaiene synthase 2 from A. crassna. The protein sequence for Delta-guaiene synthase 2 from A. crassna is accessible at UniProtKB Accession No. D0VMR7 and is provided in this disclosure as SEQ ID NO: 1.

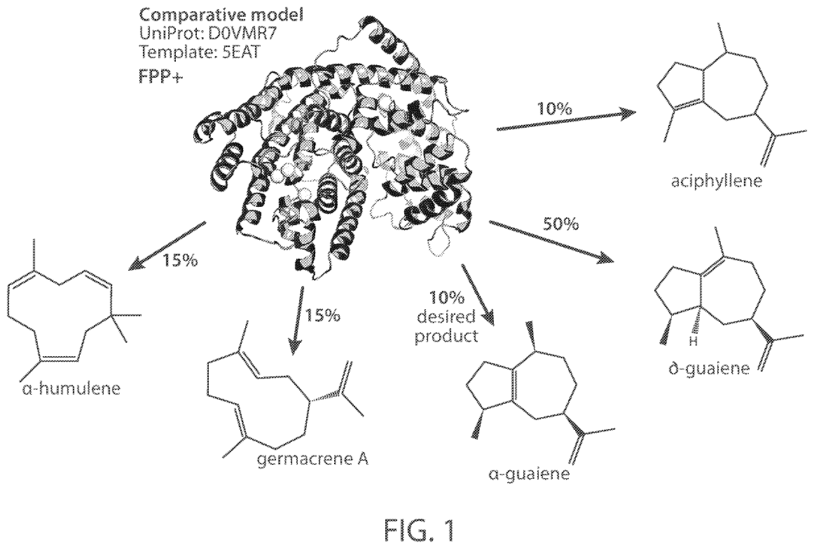

FIG. 2 depicts results from a screen of S. cerevisiae strains comprising sesquiterpene synthases that include amino acid substitutions relative to Delta-guaiene synthase 2 (SEQ ID NO: 1) as described in Example 1. A strain expressing GFP was used as a negative control. A strain expressing Delta-guaiene synthase 2 (SEQ ID NO: 1), labeled as “WT D0VMR7,” was used as a positive control. Strains for which the percentage of alpha-guaiene relative to total sesquiterpene products was higher than that of the positive control are depicted on the graph.

FIG. 3 depicts results from a screen of S. cerevisiae strains comprising sesquiterpene synthases that include amino acid substitutions relative to Delta-guaiene synthase 2 (SEQ ID NO: 1) as described in Example 2. Results from a secondary screen are shown. A strain expressing Delta-guaiene synthase 2 (SEQ ID NO: 1), labeled as “WT D0VMR7,” was used as a positive control.

FIG. 4 depicts results from a screen of S. cerevisiae strains comprising sesquiterpene synthases that include amino acid substitutions relative to Delta-guaiene synthase 2 (SEQ ID NO: 1) as described in Example 3. Results from a secondary screen are shown. A strain expressing Delta-guaiene synthase 2 (SEQ ID NO: 1), labeled as “WT D0VMR7,” was used as a positive control.

### DETAILED DESCRIPTION

Although sesquiterpenes such as guaienes are widely used in the fragrance and flavor industries, purification of sesquiterpenes from natural sources and de novo chemical synthesis often have high production costs and/or low yield. This disclosure is premised, at least in part, on the unexpected finding that amino acid substitutions in the Delta-guaiene synthase 2 protein from A. crassna (SEQ ID NO: 1) can lead to increased alpha-guaiene production relative to total sesquiterpene products or increased alpha-guaiene production relative to that of the wild type Delta-guaiene synthase of SEQ ID NO:1. Accordingly, this disclosure provides, in some aspects, enzymes that are engineered for increased production of alpha-guaiene, host cells expressing the engineered enzymes, and methods for producing alpha-guaiene using such enzymes and host cells.

### Terpenes

As used in this disclosure, unless otherwise indicated, terpenes are unsaturated hydrocarbon compounds comprising isoprene (i.e., C₅H₈) units and derivatives thereof. The terms “isoprenoid,” “terpene,” and “terpenoid” are used interchangeably in this application. For example, terpenes include pure hydrocarbons with the molecular formula (C₅H₈) n, in which n represents the number of isoprene subunits. Terpenes also include oxygenated compounds (often referred to as terpenoids). Terpenes are structurally diverse compounds and, for example, may be cyclic (e.g., monocyclic, multi-cyclic, homocyclic and heterocyclic compounds) or acyclic (e.g., linear and branched compounds). In some embodiments, a terpene may be an aroma compound. As used in this disclosure, an aroma compound refers to a compound that has characteristic scent. Any methods known in the art, including high-performance liquid chromatography (HPLC), gas chromatography (GC) (e.g., gas chromatography coupled with mass spectrometry (GC/MS) or gas chromatography coupled with flame ionization detector (GC/FID)), may be used to identify a terpene of interest.

Non-limiting examples of terpenes include monoterpenes, sesquiterpenes, diterpenes, sesterterpenes, triterpenes, and tetraterpenes. Monoterpenes comprise ten carbons. Non-limiting examples of monoterpenes include, but are not limited to, myrcene, methanol, carvone, hinokitiol, linalool, limonene, sabinene, thujene, carene, borneol, eucalyptol and camphene. Sesquiterpenes comprise 15 carbons. As used in this disclosure, sesquiterpenes include sesquiterpene hydrocarbons and sesquiterpene alcohols (sesquiterpenols). Non-limiting examples of sesquiterpenes include but are not limited to, delta-cadinene, epi-cubenol, tau-cadinol, alpha-cadinol, gamma-selinene, 10-epi-gamma-eudesmol, gamma-eudesmol, alpha/beta-eudesmol, juniper camphor, 7-epi-alpha-eudesmol, cryptomeridiol isomer 1, cryptomeridiol isomer 2, cryptomeridiol isomer 3, humulene, alpha-guaiene, delta-guaiene (alpha-bulnesene), aciphyllene, beta-guaiene, gamma-guaiene, and gamma-gurjunene, zingiberene, beta-bisabolene, beta-farnesene, beta-sesquiphellandrene, cubenol, alpha-bisabolol, alpha-curcumene, trans-nerolidol, gamma, bisabolene, beta-caryophyllene, trans-Sesquisabinene hydrate, delta-elemene, cis-eudesm-6-en-11-ol, daucene, isodaucene, trans-bergamotene, alpha-zingiberene, sesquisabinene hydrate, and 8-Isopropenyl-1,5-dimethyl-1,5-cyclodecadiene. Guaienes are one type of sesquiterpenes, which are formed from three isoprene units and often have the molecular formula C₁₅H₂₄, including compounds such as alpha-guaiene, delta-guaiene (alpha-bulnesene), aciphyllene, beta-guaiene, gamma-guaiene and gamma-gurjunene, which all have a guaiane sesquiterpene skeleton. Diterpenes comprise 20 carbons. Non-limiting examples of diterpenes include, but are not limited to, cembrene and sclareol. Sesterterpenes comprise 25 carbons. A non-limiting example of a sesterterpene is geranylfarnesol. Triterpenes comprise 30 carbons. Non-limiting examples of triterpenes include squalene, polypodatetraene, malabaricane, lanostane, cucurbitacin, hopane, oleanane, and ursolic acid. Tetraterpenes comprise 40 carbons. Non-limiting examples of tetraterpenes include carotenoids, e.g., xanthophylls and carotenes. See also, e.g., WO 2019/161141. In some embodiments, an isoprenoid is a cannabinoid. See, e.g., WO 2020/176547.

### Terpene Synthases

As used in this disclosure, a “terpene synthase” refers to a protein that is capable of producing a terpene, optionally using a prenyl diphosphate as a substrate. At least two types of terpene synthases have been characterized: classic terpene synthases and isoprenyl diphosphate synthase-type terpene synthases. Classic terpene synthases are found in prokaryotes (e.g., bacteria) and in eukaryotes (e.g., plants, fungi and amoebae), while isoprenyl diphosphate synthase-type terpene synthases have been found in insects (see, e.g., Chen et al., Proc Natl Acad Sci USA. 2016; 113 (43):12132-12137, which is hereby incorporated by reference in its entirety). Several highly conserved structural motifs have been reported in classic terpene synthases, including an aspartate-rich “DDxx (x) D/E” motif and a “NDxxSxxxD/E” motif, which have both been implicated in coordinating substrate binding (see, e.g., Starks et al., Science. 1997 Sep. 19;277 (5333): 1815-20; and Christianson et al., Curr Opin Chem Biol. 2008 April; 12 (2): 141-50, each of which is hereby incorporated by reference in its entirety). See also, e.g., WO 2019/161141 and WO 2020/176547.

Terpene synthases may be classified by the type of terpenes they produce. Terpene synthases may include, for example, monoterpene synthases, diterpene synthases, and sesquiterpene synthases. Certain non-limiting examples of monoterpene synthases and sesquiterpene synthases may be found, for example, in Degenhardt et al., Phytochemistry. 2009 October-Nov; 70 (15-16): 1621-37, which is hereby incorporated by reference in its entirety.

Monoterpene synthases catalyze the formation of 10-carbon monoterpenes. Generally, monoterpene synthases use geranyl diphosphate (GPP) as a substrate. Non-limiting examples of monoterpene synthases include Myrcene synthase (UniProtKb Accession No. O24474), (R)-limonene synthase (UniprotKB Accession No. Q2XSC6), (E)-beta-ocimene synthase (UniProtKB Accession No. Q5CD81) and Limonene synthase (UniProtKB Accession No. Q9FV72).

Diterpene synthases catalyze the formation of 20-carbon diterpenes. Generally, diterpene synthases use geranylgeranyl diphosphate as a substrate. Non-limiting examples of diterpene synthases include cis-abienol synthase (UniProtKB Accession No. H8ZM73), sclareol synthase (UniProtKB Accession No. K4HYB0) and abietadiene synthase (UniProtKB Accession No. Q38710). See, e.g., Gong et al., Nat Prod Bioprospect. 2014; 4 (2): 59-72, which is hereby incorporated by reference in its entirety.

Sesquiterpene synthases catalyze the formation of 15-carbon sesquiterpenes.

Generally, sesquiterpene synthases convert farnesyl diphosphate (FDP) into sesquiterpenes. Non-limiting examples of sesquiterpene synthases include (+)-delta-cadinene synthase (UniProtKB Accession No. Q9SANO), UniProtKB Accession No. A0A067FTE8, Beta-eudesmol synthase (UniProtKB Accession No. B1B1U4), (+)-delta-cadinene synthase isozyme XC14 (UniProtKB Accession No. Q39760), (+)-delta-cadinene synthase isozyme XC1 (UniProtKB Accession No. Q39761), (+)-delta-cadinene synthase isozyme A (UniProtKB Accession No. Q43714), Sesquiterpene synthase 2 (UniProtKB Accession No. Q9FQ26), Putative delta-guaiene synthase (UniProtKB Accession No. A0A0A0QUT9), Delta-guaiene synthase 1 (UniProtKB Accession No. D0VMR6), Alpha-zingiberene synthase (UniProtKB Accession No. Q5SBP4), (Z)-gamma-bisabolene synthase 1 (UniProtKB Accession No. Q9TOJ9), A0A067D5M4, Delta-elemene synthase (UniProtKB Accession No. A0A097ZIE0), ShoBecSQTS1, A0A068UHT0, terpene synthase (UniProtKB Accession No. G5CV47), A0A068VE40 and A0A068VI46.

The present disclosure also encompasses sesquiterpene synthases that are multi-functional (e.g., capable of producing more than one sesquiterpene). In some embodiments, a sesquiterpene synthase is capable of producing delta-cadinene and alpha-cadinol. In some embodiments, a sesquiterpene synthase is capable of producing delta-cadinene, tau-cadinol, and alpha-cadinol. In some embodiments, a sesquiterpene synthase is capable of producing alpha-guaiene and delta-guaiene. In some embodiments, a sesquiterpene synthase is capable of producing beta-caryophyllene and humulene. In some embodiments, a sesquiterpene synthase is capable of producing alpha-guaiene, delta-guaiene, beta-elemene, neointermediol, and/or humulene. Beta-elemene is further described in and incorporated by reference from WO2005/052163. In some embodiments, a sesquiterpene synthase is capable of producing aciphyllene. In some embodiments, a sesquiterpene synthase is capable of producing alpha-guaiene and aciphyllene. In some embodiments, the most abundant sesquiterpene produced by a sesquiterpene synthase is alpha-guaiene and the second most abundant sesquiterpene produced by the sesquiterpene synthases is aciphyllene.

The present disclosure also encompasses fragments of sesquiterpene synthases. As used in this disclosure, a fragment of a sesquiterpene synthase refers to a portion of a sesquiterpene synthase that is smaller than the full-length molecule. A fragment of a sesquiterpene synthase of the disclosure may include a biologically active portion of the enzyme, such as a catalytic domain. A functional fragment of a sesquiterpene synthase refers to a fragment of a sesquiterpene synthase that has the same type of activity as the full length sesquiterpene synthase, although the level of activity of the fragment may vary compared to the level of activity of the full length sesquiterpene synthase.

### Production of Alpha-Guaiene

Guaienes are sesquiterpenes with the molecular formula C₁₅H₂₄. Non-limiting examples of guaienes include alpha-guaiene (α-guaiene; CAS Registry Number 3691 Dec. 1), beta-guaiene (β-guaiene; CAS Registry Number 88-84-6), alpha-bulnesene (also known as delta-guaiene (δ-guaiene; CAS Registry Number 3691-11-0), gamma guaiene (γ-guaiene; CAS Registry Number 145267-53-4), aciphyllene (CAS Registry Number 87745-31-1) and gamma-gurjunene (γ-gurjunene; CAS Registry Number 22567-17-5).

In some embodiments, a sesquiterpene synthase associated with the disclosure is the Delta-guaiene synthase 2 protein from A. crassna. The amino acid sequence of the Delta-guaiene synthase 2 protein from A. crassna is accessible through UniProtKB Accession No. D0VMR7, and is provided here as SEQ ID NO: 1:

(SEQ ID NO: 1)
MSSAKLGSASEDVSRRDANYHPTVWGDFFLTHSSNFLENNDSILEKHEELKQEVRNLLVV
ETSDLPSKIQLTDEIIRLGVGYHFETEIKAQLEKLHDHQLHLNFDLLTTSVWFRLLRGHG
FSIPSDVFKRFKNTKGEFETEDARTLWCLYEATHLRVDGEDILEEAIQFSRKRLEALLPK
LSFPLSECVRDALHIPYHRNVQRLAARQYIPQYDAEQTKIESLSLFAKIDFNMLQALHQS
ELREASRWWKEFDFPSKLPYARDRIAEGYYWMMGAHFEPKFSLSRKFLNRIVGITSLIDD
TYDVYGTLEEVTLFTEAVERWDIEAVKDIPKYMQVIYIGMLGIFEDFKDNLINARGKDYC
IDYAIEVFKEIVRSYQREAEYFHTGYVPSYDEYMENSIISGGYKMFIILMLIGRGEFELK
ETLDWASTIPEMVKASSLIARYIDDLQTYKAEEERGETVSAVRCYMREFGVSEEQACKKM
REMIEIEWKRLNKTTLEADEISSSVVIPSLNFTRVLEVMYDKGDGYSDSQGVTKDRIAAL
LRHAIEI

A non-limiting example of a nucleic acid sequence encoding SEQ ID NO: 1 is provided here as SEQ ID NO: 2.

(SEQ ID NO: 2)
atgtcttcagctaaactgggaagtgcctccgaggacgtttctcgtcgggatgcaaattacca
ccctactgtatggggtgatttctttttgacgcatagctccaacttcctcgaaaacaatgact
cgatccttgaaaagcacgaagaattgaagcaggaagtgaggaacctattagtcgttgaaaca
tctgacttgccatctaaaattcaattgaccgatgagataattagattgggtgttggctatca
ttttgaaactgaaatcaaggctcaattagaaaagttgcacgaccaccaattgcatttaaact
tcgatcttttgaccacttcggtctggttcagacttttgcgaggtcacggtttttccattcca
tcagatgtttttaagagattcaaaaacaccaagggtgaatttgagactgaagacgcgagaac
attatggtgtttgtacgaagccacccatcttagagttgacggggaggatatcctagaagagg
ctatacaattctctcgtaagagattggaagctctgttacccaaattgagcttcccattgtcc
gaatgcgtcagagatgctctgcatattccttaccacagaaatgtacaaagattggccgctag
acaatatatcccacaatacgatgctgaacagactaagattgaatctctatctttattcgcca
agatcgactttaacatgttgcaagctttgcaccaaagtgaactgagagaagcctcacgctggt
ggaaagaatttgacttcccatccaagttaccttacgcaagggatagaattgctgaaggttatt
actggatgatgggtgctcattttgaaccgaagttctctttgtctcgtaagttcttgaatagaa
ttgtcggtatcacttcgttaattgatgacacgtatgatgtttacgggacccttgaagaggtga
cactattcactgaagcggtcgaacgttgggacattgaggcagttaaagatatcccaaagtaca
tgcaagtcatctacatcggtatgttgggtatctttgaagactttaaagataacttaattaatg
ctcgaggtaaagattactgtatcgactatgctattgaagtctttaaggaaattgttcgttcct
atcaaagagaagctgaatatttccacaccggctacgttccaagttacgatgaatacatggaga
atagtataatatctggaggttacaagatgttcattattcttatgttgattggtagaggggaat
ttgaattgaaggaaacactagactgggcctcaactattcctgaaatggttaaggctagttctc
tcattgctagatacatcgacgatctccagacctataaggcagaggaggaaagaggtgaaactg
tatcagctgttagatgttacatgagagaatttggtgtgtctgaggaacaagcttgcaagaaaa
tgcgtgaaatgatcgaaattgaatggaagcgattgaacaagacgactttggaggcagacgaaa
tatcctccagtgttgtaataccatctctcaacttcaccagagttttggaagtcatgtatgaca
aaggtgatggttactctgattctcaaggtgtcacaaaggatagaatcgccgctttacttagac
acgcaattgaaatc

Other non-limiting examples of sesquiterpene synthases include those associated with UniProt Accession No. D0VMR6, UniProt Accession No. D0VMR8, and UniProt Accession No. Q49SP3.

A sesquiterpene synthase may comprise a sequence that is at least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to SEQ ID NO: 1 or a conservatively substituted version thereof. In certain embodiments, a sesquiterpene synthase comprises SEQ ID NO: 1 or a conservatively substituted version thereof. In certain embodiments a sesquiterpene synthase consists of or consists essentially of SEQ ID NO: 1 or a conservatively substituted version thereof.

### Sesquiterpene Synthase Variants for Increased Production of Alpha Guaiene

Naturally occurring sesquiterpene synthases that produce guaienes generally produce a mixture of different sesquiterpene products. For example, as shown in Example 1, expression of the naturally occurring Delta-guaiene synthase 2 protein from A. crassna (SEQ ID NO: 1) in a host cell led to production of a mixture of sesquiterpene products, which included ˜14.6% alpha-guaiene as determined using gas chromatography (GC) (Table 4).

It would be advantageous to be able to influence the amount or ratio of different guaiene products produced by a sesquiterpene synthase. In particular, given the importance of alpha-guaiene for fragrance and flavoring, it would be advantageous to be able to produce increased amounts of alpha-guaiene or increased amounts of alpha-guaiene relative to other sesquiterpene products. Aspects of the disclosure relate to the surprising identification of synthetic sesquiterpene synthases that produce increased amounts of alpha-guaiene, or increased ratios of alpha-guaiene relative to total sesquiterpene products, compared to that produced by a control sesquiterpene synthase. In some embodiments, a control sesquiterpene synthase comprises the sequence of SEQ ID NO: 1. In some embodiments, sesquiterpene synthases associated with the disclosure produce more alpha-guaiene than one or more other sesquiterpene products, such as delta-guaiene. As shown in Examples 1-3, it was surprisingly found that variant sesquiterpene synthases that included amino acid substitutions relative to SEQ ID NO: 1 produced an increased percentage of alpha-guaiene relative to total sesquiterpene products compared to a sesquiterpene synthase that comprises the sequence of SEQ ID NO: 1 (Tables 4, 6 and 7).

In some embodiments, the sequence of a sesquiterpene synthase associated with the disclosure comprises one or more amino acid substitutions relative to SEQ ID NO: 1, wherein the one or more amino acid substitutions are at one or more positions corresponding to position 23, 44, 72, 86, 111, 118, 122, 124, 134, 147, 153, 188, 191, 201, 205, 212, 217, 224, 252, 255, 274, 275, 289, 290, 291, 292, 293, 295, 301, 346, 368, 381, 390, 398, 404, 406, 407, 419, 433, 442, 443, 448, 458, 467, 480, 494, 499, 507, 509, 512, 516, 519, 526, 533, and/or 542 in SEQ ID NO: 1.

In some embodiments, the sequence of a sesquiterpene synthase associated with the disclosure comprises one or more amino acid substitutions relative to SEQ ID NO: 1, wherein at least one of the amino acid substitutions is at a position corresponding to position 44, 122, 212, 217, 274, 275, 293, 295, 301, 368, 398, 404, 407, 507, 515, 525, 533, and/or 542 in SEQ ID NO: 1. In some embodiments, the sequence of the sesquiterpene synthase further comprises at least one amino acid substitution at a position corresponding to position 23, 72, 86, 111, 118, 124, 134, 147, 153, 188, 191, 201, 205, 224, 252, 255, 289, 290, 291, 292, 346, 381, 390, 406, 419, 433, 442, 443, 448, 458, 467, 480, 494, 499, 509, 512, 516, 519, and/or 526 in SEQ ID NO: 1.

In some embodiments, the sequence of a sesquiterpene synthase associated with the disclosure comprises two or more amino acid substitutions relative to SEQ ID NO: 1, wherein:
(i) at least one of the amino acid substitutions is at a position corresponding to position 122, 274, 275, 293, 295, 301, 368, 398, 404, 407, 507, 515, 525, and/or 533 in SEQ ID NO: 1; and
(ii) at least one of the amino acid substitutions is at a position corresponding to position 72, 124, 153, 191, 201, 205, 289, 290, 291, 292, 346, 406, 442, 480, 494, 509, 512, and/or 526 in SEQ ID NO: 1.

In some embodiments, the sequence of a sesquiterpene synthase associated with the disclosure comprises two or more amino acid substitutions relative to SEQ ID NO: 1, wherein the two or more amino acid substitutions are at positions corresponding to position 72, 122, 124, 153, 191, 201, 205, 274, 275, 289, 290, 291, 292, 293, 295, 301, 346, 368, 398, 404, 406, 407, 442, 480, 494, 507, 509, 512, 526 and/or 533 in SEQ ID NO: 1.

While one or more amino acid substitutions are described in this disclosure relative to the sequence of SEQ ID NO: 1, it should be appreciated that the disclosure encompasses the same one or more substitutions at corresponding residues in other sesquiterpene synthases sequences. One of ordinary skill in the art would be able to determine which one or more residues in a different sesquiterpene synthase correspond to any one or more residues in the sequence of SEQ ID NO: 1. As used in this application, a residue (such as a nucleic acid residue or an amino acid residue) in a first sequence is referred to as corresponding to a position or residue (such as a nucleic acid residue or an amino acid residue) in a second sequence when the residue in the first sequence is at the counterpart position of the residue in the second sequence when the first and the second sequences are aligned using amino acid sequence alignment tools known in the art.

For example, SEQ ID NO: 1 has a valine (V) residue at amino acid position 292. Some sesquiterpene synthases have an isoleucine (I) residue at the corresponding position instead of a V residue. It should be appreciated that in sesquiterpene synthases that have an I residue at the position corresponding to amino acid 292 in SEQ ID NO: 1, the amino acid substitutions disclosed herein for that position are still applicable. Similarly, SEQ ID NO: 1 has a serine(S) residue at amino acid position 296. Some sesquiterpene synthases have a proline (P) residue at the corresponding position instead of an S residue. It should be appreciated that in sesquiterpene synthases that have a P residue at the position corresponding to amino acid 296 in SEQ ID NO: 1, the amino acid substitutions disclosed herein (with the exception of an S to P substitution) for that position are still applicable.

In some embodiments, a sesquiterpene synthase comprises: an aspartate (D) residue at a position corresponding to position 23 in SEQ ID NO: 1; a valine (V) residue at a position corresponding to position 44 in SEQ ID NO: 1; an isoleucine (I) residue at a position corresponding to position 72 in SEQ ID NO: 1; a glutamate (E) residue at a position corresponding to position 86 in SEQ ID NO: 1; a leucine (L) residue at a position corresponding to position 111 in SEQ ID NO: 1; a glutamine (Q) residue at a position corresponding to position 118 in SEQ ID NO: 1; an asparagine (N) residue at a position corresponding to position 122 in SEQ ID NO: 1; a serine(S) residue at a position corresponding to position 124 in SEQ ID NO: 1; a D residue at a position corresponding to position 134 in SEQ ID NO: 1; a V residue at a position corresponding to position 147 in SEQ ID NO: 1; an L residue at a position corresponding to position 147 in SEQ ID NO: 1; an S residue at a position corresponding to position 153 in SEQ ID NO: 1; a Q residue at a position corresponding to position 188 in SEQ ID NO: 1; an N residue at a position corresponding to position 191 in SEQ ID NO: 1; an S residue at a position corresponding to position 201 in SEQ ID NO: 1; an I residue at a position corresponding to position 201 of SEQ ID NO: 1; an E residue at a position corresponding to position 205 in SEQ ID NO: 1; a phenylalanine (F) residue at a position corresponding to position 212 in SEQ ID NO: 1; a E residue at a position corresponding to position 217 in SEQ ID NO: 1; an L residue at a position corresponding to position 224 in SEQ ID NO: 1; an L residue at a position corresponding to position 252 in SEQ ID NO: 1; an alanine (A) residue at a position corresponding to position 255 in SEQ ID NO: 1; an S residue at a position corresponding to position 274 in SEQ ID NO: 1; a glycine (G) residue at a position corresponding to position 275 in SEQ ID NO: 1; a lysine (K) residue at a position corresponding to position 289 in SEQ ID NO: 1; a histidine (H) residue at a position corresponding to position 289 in SEQ ID NO: 1; an H residue at a position corresponding to position 290 in SEQ ID NO: 1; a K residue at a position corresponding to position 290 in SEQ ID NO: 1; a V residue at a position corresponding to position 291 in SEQ ID NO: 1; a F or methionine (M) residue at a position corresponding to position 292 in SEQ ID NO: 1; a cysteine (C) residue at a position corresponding to position 292 in SEQ ID NO: 1; a Q, I, or N residue at a position corresponding to position 293 in SEQ ID NO: 1; an I residue at a position corresponding to position 295 in SEQ ID NO: 1; a V residue at a position corresponding to position 295 in SEQ ID NO: 1; an S residue at a position corresponding to position 301 in SEQ ID NO: 1; an E residue at a position corresponding to position 346 in SEQ ID NO: 1; a C residue at a position corresponding to position 368 in SEQ ID NO: 1; a tryptophan (W) residue at a position corresponding to position 381 in SEQ ID NO: 1; a F residue at a position corresponding to position 390 in SEQ ID NO: 1; a C residue at a position corresponding to position 398 in SEQ ID NO: 1; a W residue at a position corresponding to position 404 in SEQ ID NO: 1; an L residue at a position corresponding to position 406 in SEQ ID NO: 1; a G residue at a position corresponding to position 407 in SEQ ID NO: 1; a S residue at a position corresponding to position 419 in SEQ ID NO: 1; a I residue at a position corresponding to position 433 in SEQ ID NO: 1; an L residue at a position corresponding to position 433 in SEQ ID NO: 1; an L residue at a position corresponding to position 442 in SEQ ID NO: 1; a W residue at a position corresponding to position 443 in SEQ ID NO: 1; a threonine (T) residue at a position corresponding to position 443 in SEQ ID NO: 1; an N residue at a position corresponding to position 444 in SEQ ID NO: 1; a G residue at a position corresponding to position 448 in SEQ ID NO: 1; an L residue at a position corresponding to position 458 in SEQ ID NO: 1; a V residue at a position corresponding to position 458 in SEQ ID NO: 1; a K residue at a position corresponding to position 467 in SEQ ID NO: 1; an I residue at a position corresponding to position 480 in SEQ ID NO: 1; an E residue at a position corresponding to position 494 in SEQ ID NO: 1; an N residue at a position corresponding to position 499 in SEQ ID NO: 1; a W residue at a position corresponding to position 507 in SEQ ID NO: 1; an A residue at a position corresponding to position 509 in SEQ ID NO: 1; an L residue at a position corresponding to position 512 in SEQ ID NO: 1; an M residue at a position corresponding to position 515 in SEQ ID NO: 1; an A residue at a position corresponding to position 516 in SEQ ID NO: 1; a V residue at a position corresponding to position 516 in SEQ ID NO: 1; an L residue at a position corresponding to position 519 in SEQ ID NO: 1; an F residue at a position corresponding to position 526 in SEQ ID NO: 1; an N residue at a position corresponding to position 533 in SEQ ID NO: 1; a V residue at a position corresponding to position 542 in SEQ ID NO: 1; or any combination thereof.

In some embodiments, a sesquiterpene synthase comprises: a D residue at a position corresponding to position 23 in SEQ ID NO: 1; a V residue at a position corresponding to position 44 in SEQ ID NO: 1; an I residue at a position corresponding to position 72 in SEQ ID NO: 1; an E residue at a position corresponding to position 86 in SEQ ID NO: 1; a L residue at a position corresponding to position 111 in SEQ ID NO: 1; a Q residue at a position corresponding to position 118 in SEQ ID NO: 1; an N residue at a position corresponding to position 122 in SEQ ID NO: 1; an S residue at a position corresponding to position 124 in SEQ ID NO: 1; a D residue at a position corresponding to position 134 in SEQ ID NO: 1; a V or L residue at a position corresponding to position 147 in SEQ ID NO: 1; an S residue at a position corresponding to position 153 in SEQ ID NO: 1; a Q residue at a position corresponding to position 188 in SEQ ID NO: 1; an N residue at a position corresponding to position 191 in SEQ ID NO: 1; an I or S residue at a position corresponding to position 201 in SEQ ID NO: 1; an E residue at a position corresponding to position 205 in SEQ ID NO: 1; a F residue at a position corresponding to position 212 in SEQ ID NO: 1; a E residue at a position corresponding to position 217 in SEQ ID NO: 1; a L residue at a position corresponding to position 224 in SEQ ID NO: 1; a L residue at a position corresponding to position 252 in SEQ ID NO: 1; an A residue at a position corresponding to position 255 in SEQ ID NO: 1; an S residue at a position corresponding to position 274 in SEQ ID NO: 1; a G residue at a position corresponding to position 275 in SEQ ID NO: 1; an L, T, S, H, M, K or D residue at a position corresponding to position 289 in SEQ ID NO: 1; a K or H residue at a position corresponding to position 290 in SEQ ID NO: 1; an F, L, T, V or C residue at a position corresponding to position 291 in SEQ ID NO: 1; an A, Q, C, Y, H, E, F, M, W, T or F residue at a position corresponding to position 292 in SEQ ID NO: 1; an L, V, T, Y, C, F, W, Q, I, N, or M residue at a position corresponding to position 293 in SEQ ID NO: 1; an E, D, N, W, G, V or I residue at a position corresponding to position 295 in SEQ ID NO: 1; an S residue at a position corresponding to position 301 in SEQ ID NO: 1; an E residue at a position corresponding to position 346 in SEQ ID NO: 1; a C residue at a position corresponding to position 368 in SEQ ID NO: 1; a W residue at a position corresponding to position 381 in SEQ ID NO: 1; a F residue at a position corresponding to position 390 in SEQ ID NO: 1; a C residue at a position corresponding to position 398 in SEQ ID NO: 1; a W residue at a position corresponding to position 404 in SEQ ID NO: 1; an L, N, W, or T residue at a position corresponding to position 406 in SEQ ID NO: 1; a G residue at a position corresponding to position 407 in SEQ ID NO: 1; a S residue at a position corresponding to position 419 in SEQ ID NO: 1; an I or L residue at a position corresponding to position 433 in SEQ ID NO: 1; an L residue at a position corresponding to position 442 in SEQ ID NO: 1; a W or T residue at a position corresponding to position 443 in SEQ ID NO: 1; an N residue at a position corresponding to position 444 in SEQ ID NO: 1; a G residue at a position corresponding to position 448 in SEQ ID NO: 1; a L or V residue at a position corresponding to position 458 in SEQ ID NO: 1; a K residue at a position corresponding to position 467 in SEQ ID NO: 1; an I residue at a position corresponding to position 480 in SEQ ID NO: 1; an E residue at a position corresponding to position 494 in SEQ ID NO: 1; an N residue at a position corresponding to position 499 in SEQ ID NO: 1; a W residue at a position corresponding to position 507 in SEQ ID NO: 1; an A residue at a position corresponding to position 509 in SEQ ID NO: 1; an L residue at a position corresponding to position 512 in SEQ ID NO: 1; a M residue at a position corresponding to position 515 in SEQ ID NO: 1; an A or V residue at a position corresponding to position 516 in SEQ ID NO: 1; a L residue at a position corresponding to position 519 in SEQ ID NO: 1; an F residue at a position corresponding to position 526 of SEQ ID NO: 1; an N residue at a position corresponding to position 533 of SEQ ID NO: 1; a V residue at a position corresponding to position 542 in SEQ ID NO: 1; or any combination thereof.

In some embodiments, a sesquiterpene synthase comprises the following amino acid substitutions relative to SEQ ID NO: 1: N289H, V292F, G293N, T295V, K404W, F406L, and F512L; N289H, 1291V, V292F, G293Q, T295V, K404W, F406L, and F512L; N289H, F406L, F512L, D191N, and A205E; 1291V, V292F, G293Q, T295V, K404W, F406L, 1507W, and F512L; N289H, V292F, T295V, K404W, F406L, and F512L; N289H, 1291V, V292F, G293N, T295V, K404W, F406L, and F512L; V292F, G293Q, K404W, and F406L; N289H, V292F, G293I, T295V, F406L, 1407G, and F512L; V292F, G293Q, K404W, F406L, 1407G, and F512L; N289H, 1291V, V292F, G2931, T295V, F406L, 1407G, and F512L; V292F, T295V, K404W, F406L, 1507W, and F512L; N289H, V292M, G293I, F406L, F512L, and M480I; N289H, 1291V, V292F, G293Q, T295V, K404W, and F406L; V292F, G293Q, T295V, F406L, and F512L; V292F, T295V, K404W, F406L, and F512L; 1291V, V292F, G293I, K404W, F406L, and I507W; V292F, G293I, T295V, F406L, 1407G, and F512L; N289H, V292F, G2931, T295V, F406L, 1507W, and F512L; N289H, 1291V, V292F, G2931, T295V, K404W, F406L, and F512L; N289H, V292F, G293Q, T295V, F406L, 1407G, and F512L; N289H, 1291V, V292F, G293N, T295V, K404W, and F406L; N289H, V292F, G293Q, F406L, 1507W, and F512L; 1291V, V292F, G293N, T295V, K404W, F406L, 1507W, and F512L; N289H, V292F, G293N, K404W, F406L, 1507W, and F512L; N289H, V292F, G2931, T295V, F406L, 1407G, 1507W, and F512L; N289H, 1291V, V292F, G293N, T295V, F406L, and F512L; 1291V, V292F, G293Q, K404W, F406L, 1407G, and F512L; I291V, V292F, G293Q, T295V, K404W, F406L, 1407G, and F512L; N289H, V292F, T295V, F406L, and F512L; N289H, V292F, K404W, F406L, and F512L; V292F, G293N, T295V, F406L, and F512L; N289H, 1291V, V292F, G293N, T295V, F406L, 1407G, 1507W, and F512L; N289H, 1291V, V292F, G293I, T295V, F406L, and F512L; N289H, 1291V, V292F, G293Q, T295V, F406L, and I507W; N289H, V292F, G293I, F406L, 1407G, 1507W, and F512L; N289H, V292F, G293Q, T295V, K404W, F406L, and I507W; I291V, V292F, G293Q, T295V, F406L, 1507W, and F512L; S122N, N289H, 1291V, V292F, G293Q, T295V, K404W, F406L, and F512L; P124S, N289H, 1291V, V292F, G293Q, T295V, K404W, F406L, and F512L; T153S, N289H, 1291V, V292F, G293Q, T295V, D346E, K404W, F406L, and F512L; P124S, N289H, 1291V, V292F, G293Q, T295V, K404W, and F406L; R290K, V292F, T295V, K404W, F406L, and F512L; N289H, 1291V, V292F, G293Q, T295V, K404W, F406L, Y442L, and F512L; N289H, V292F, G293N, T295V, K404W, F406L, Y442L, and F512L; V292F, G293Q, and K404W; T721, N289H, 1291V, V292F, G293Q, T295V, K404W, F406L, T494E, and F512L; T72I, N289H, V292F, T295V, K404W, F406L, T494E, and F512L; T721, N289H, V292F, G2931, T295V, F406L, 1407G, Y442L, 1507W, and F512L; A275G, G293W, 1294V, T295W, T301S, F368C, S509A, F512L, Y526F, and T533N; P124S, V292F, T295V, K404W, F406L, 1507W, and F512L; N289H, V292F, T295V, K404W, F406L, Y442L, and F512L; N289H, F406L, F512L, and T295V; N289H, F406L, F512L, T295V, and G274S; N289H, F406L, F512L, T295V, and V201I; N289H, F406L, F512L, T295V, and 1398C; T721, N289H, R290K, 1291V, V292F, G293Q, T295V, K404W, F406L, Y442L, T494E, and F512L; L44V, T72I, N289H, I291V, V292F, G293Q, T295V, K404W, F406L, Y442L, and F512L; L44V, T72I, T86E, G118Q, T134D, W147L, Q212F, Q217E, S224L, F252L, P255A, N289H, R290K, 1291V, V292F G293Q, T295V, D346E, Y390F, K404W, F406L, L419S, Y442L, T458V, R467K, F512L, and R542V; T23D, L44V, T72I, N289H, 1291V, V292F, G293Q, T295V, K404W, F406L, Y442L, and F512L; V201S, N289H, 1291V, V292F, G293N, T295V, Y381W, K404W, F406L, T494E, and F512L; V201S, V292C, T2951, and D444N; V201S, V292C, T295I, D444N, and L516V; L44V, T72I, V201S, V292C, T2951, D444N, and L516V; L44V, T72I, V201S, V292C, T295I, D444N, and L516A; L44V, T72I, V201S, V292C, T295I, 1443W, and D444N; L44V, T72I, V201S, V292C, T295I, D444N, and M519L; W147V, C188Q, V201S, S224L, V292C, T295I, and D444N; L44V, T72I, V111L, V201S, S224L, V292C, T2951, 1443T, D444N, T448G, and M519L; L44V, T72I, V111L, V201S, V292C, T295I, V4331, 1443T, D444N, T448G, D499N, and L516V; L44V, T72I, V201S, V292C, T295I, D444N, L516V, and M519L; L44V, T72I, V201S, V292C, T295I, D444N, V515M, and L516V; V111L, V201S, S224L, V292C, T2951, 1443T, D444N, and L516V; V201S, V292C, T2951, D444N, and L516V; V201S, V292C, T2951, D444N, T458L, and L516A; V201S, V292C, T2951, 1443T, D444N, and L516V; V201S, V292C, T295I, V433I, D444N, and L516V; or V201S, V292C, T295I, V433L, D444N, and L516V.

In some embodiments, the sesquiterpene synthase that comprises the amino acid substitutions N289H, V292F, G293N, T295V, K404W, F406L, and F512L relative to SEQ ID NO: 1 comprises the sequence of SEQ ID NO: 3; the sesquiterpene synthase that comprises the amino acid substitutions N289H, 1291V, V292F, G293Q, T295V, K404W, F406L, and F512L relative to SEQ ID NO: 1 comprises the sequence of SEQ ID NO: 4; the sesquiterpene synthase that comprises the amino acid substitutions N289H, F406L, F512L, D191N, and A205E relative to SEQ ID NO: 1 comprises the sequence of SEQ ID NO: 5; the sesquiterpene synthase that comprises the amino acid substitutions 1291V, V292F, G293Q, T295V, K404W, F406L, 1507W, and F512L relative to SEQ ID NO: 1 comprises the sequence of SEQ ID NO: 6; the sesquiterpene synthase that comprises the amino acid substitutions N289H, V292F, T295V, K404W, F406L, and F512L relative to SEQ ID NO: 1 comprises the sequence of SEQ ID NO: 7; the sesquiterpene synthase that comprises the amino acid substitutions N289H, 1291V, V292F, G293N, T295V, K404W, F406L, and F512L relative to SEQ ID NO: 1 comprises the sequence of SEQ ID NO: 8; the sesquiterpene synthase that comprises the amino acid substitutions V292F, G293Q, K404W, and F406L relative to SEQ ID NO: 1 comprises the sequence of SEQ ID NO: 9; the sesquiterpene synthase that comprises the amino acid substitutions N289H, V292F, G2931, T295V, F406L, 1407G, and F512L relative to SEQ ID NO: 1 comprises the sequence of SEQ ID NO: 10; the sesquiterpene synthase that comprises the amino acid substitutions V292F, G293Q, K404W, F406L, 1407G, and F512L relative to SEQ ID NO: 1 comprises the sequence of SEQ ID NO: 11; the sesquiterpene synthase that comprises the amino acid substitutions N289H, 1291V, V292F, G293I, T295V, F406L, 1407G, and F512L relative to SEQ ID NO: 1 comprises the sequence of SEQ ID NO: 12; the sesquiterpene synthase that comprises the amino acid substitutions V292F, T295V, K404W, F406L, 1507W, and F512L relative to SEQ ID NO: 1 comprises the sequence of SEQ ID NO: 13; the sesquiterpene synthase that comprises the amino acid substitutions N289H, V292M, G2931, F406L, F512L, and M480I relative to SEQ ID NO: 1 comprises the sequence of SEQ ID NO: 14; the sesquiterpene synthase that comprises the amino acid substitutions N289H, 1291V, V292F, G293Q, T295V, K404W, and F406L relative to SEQ ID NO: 1 comprises the sequence of SEQ ID NO: 15; the sesquiterpene synthase that comprises the amino acid substitutions V292F, G293Q, T295V, F406L, and F512L relative to SEQ ID NO: 1 comprises the sequence of SEQ ID NO: 16; the sesquiterpene synthase that comprises the amino acid substitutions V292F, T295V, K404W, F406L, and F512L relative to SEQ ID NO: 1 comprises the sequence of SEQ ID NO: 17; the sesquiterpene synthase that comprises the amino acid substitutions I291V, V292F, G293I, K404W, F406L, and 1507W relative to SEQ ID NO: 1 comprises the sequence of SEQ ID NO: 18; the sesquiterpene synthase that comprises the amino acid substitutions V292F, G2931, T295V, F406L, 1407G, and F512L relative to SEQ ID NO: 1 comprises the sequence of SEQ ID NO: 19; the sesquiterpene synthase that comprises the amino acid substitutions N289H, V292F, G2931, T295V, F406L, 1507W, and F512L relative to SEQ ID NO: 1 comprises the sequence of SEQ ID NO: 20; the sesquiterpene synthase that comprises the amino acid substitutions N289H, 1291V, V292F, G2931, T295V, K404W, F406L, and F512L relative to SEQ ID NO: 1 comprises the sequence of SEQ ID NO: 21; the sesquiterpene synthase that comprises the amino acid substitutions N289H, V292F, G293Q, T295V, F406L, 1407G, and F512L relative to SEQ ID NO: 1 comprises the sequence of SEQ ID NO: 22; the sesquiterpene synthase that comprises the amino acid substitutions N289H, 1291V, V292F, G293N, T295V, K404W, and F406L relative to SEQ ID NO: 1 comprises the sequence of SEQ ID NO: 23; the sesquiterpene synthase that comprises the amino acid substitutions N289H, V292F, G293Q, F406L, 1507W, and F512L relative to SEQ ID NO: 1 comprises the sequence of SEQ ID NO: 24; the sesquiterpene synthase that comprises the amino acid substitutions I291V, V292F, G293N, T295V, K404W, F406L, 1507W, and F512L relative to SEQ ID NO: 1 comprises the sequence of SEQ ID NO: 25; the sesquiterpene synthase that comprises the amino acid substitutions N289H, V292F, G293N, K404W, F406L, 1507W, and F512L relative to SEQ ID NO: 1 comprises the sequence of SEQ ID NO: 26; the sesquiterpene synthase that comprises the amino acid substitutions N289H, V292F, G293I, T295V, F406L, 1407G, 1507W, and F512L relative to SEQ ID NO: 1 comprises the sequence of SEQ ID NO: 27; the sesquiterpene synthase that comprises the amino acid substitutions N289H, 1291V, V292F, G293N, T295V, F406L, and F512L relative to SEQ ID NO: 1 comprises the sequence of SEQ ID NO: 28; the sesquiterpene synthase that comprises the amino acid substitutions I291V, V292F, G293Q, K404W, F406L, 1407G, and F512L relative to SEQ ID NO: 1 comprises the sequence of SEQ ID NO: 29; the sesquiterpene synthase that comprises the amino acid substitutions 1291V, V292F, G293Q, T295V, K404W, F406L, 1407G, and F512L relative to SEQ ID NO: 1 comprises the sequence of SEQ ID NO: 30; the sesquiterpene synthase that comprises the amino acid substitutions N289H, V292F, T295V, F406L, and F512L relative to SEQ ID NO: 1 comprises the sequence of SEQ ID NO: 31; the sesquiterpene synthase that comprises the amino acid substitutions N289H, V292F, K404W, F406L, and F512L relative to SEQ ID NO: 1 comprises the sequence of SEQ ID NO: 32; the sesquiterpene synthase that comprises the amino acid substitutions V292F, G293N, T295V, F406L, and F512L relative to SEQ ID NO: 1 comprises the sequence of SEQ ID NO: 33; the sesquiterpene synthase that comprises the amino acid substitutions N289H, 1291V, V292F, G293N, T295V, F406L, 1407G, 1507W, and F512L relative to SEQ ID NO: 1 comprises the sequence of SEQ ID NO: 34; the sesquiterpene synthase that comprises the amino acid substitutions N289H, 1291V, V292F, G2931, T295V, F406L, and F512L relative to SEQ ID NO: 1 comprises the sequence of SEQ ID NO: 35; the sesquiterpene synthase that comprises the amino acid substitutions N289H, 1291V, V292F, G293Q, T295V, F406L, and 1507W relative to SEQ ID NO: 1 comprises the sequence of SEQ ID NO: 36; the sesquiterpene synthase that comprises the amino acid substitutions N289H, V292F, G2931, F406L, 1407G, 1507W, and F512L relative to SEQ ID NO: 1 comprises the sequence of SEQ ID NO: 37; the sesquiterpene synthase that comprises the amino acid substitutions N289H, V292F, G293Q, T295V, K404W, F406L, and I507W relative to SEQ ID NO: 1 comprises the sequence of SEQ ID NO: 38; the sesquiterpene synthase that comprises the amino acid substitutions I291V, V292F, G293Q, T295V, F406L, 1507W, and F512L relative to SEQ ID NO: 1 comprises the sequence of SEQ ID NO: 39; the sesquiterpene synthase that comprises the amino acid substitutions S122N, N289H, 1291V, V292F, G293Q, T295V, K404W, F406L, and F512L relative to SEQ ID NO: 1 comprises the sequence of SEQ ID NO: 84; the sesquiterpene synthase that comprises the amino acid substitutions P124S, N289H, 1291V, V292F, G293Q, T295V, K404W, F406L, and F512L relative to SEQ ID NO: 1 comprises the sequence of SEQ ID NO: 80; the sesquiterpene synthase that comprises the amino acid substitutions T153S, N289H, 1291V, V292F, G293Q, T295V, D346E, K404W, F406L, and F512L relative to SEQ ID NO: 1 comprises the sequence of SEQ ID NO: 82; the sesquiterpene synthase that comprises the amino acid substitutions P124S, N289H, 1291V, V292F, G293Q, T295V, K404W, and F406L relative to SEQ ID NO: 1 comprises the sequence of SEQ ID NO: 81; the sesquiterpene synthase that comprises the amino acid substitutions R290K, V292F, T295V, K404W, F406L, and F512L relative to SEQ ID NO: 1 comprises the sequence of SEQ ID NO: 83; the sesquiterpene synthase that comprises the amino acid substitutions N289H, 1291V, V292F, G293Q, T295V, K404W, F406L, Y442L, and F512L relative to SEQ ID NO: 1 comprises the sequence of SEQ ID NO: 78; the sesquiterpene synthase that comprises the amino acid substitutions N289H, V292F, G293N, T295V, K404W, F406L, Y442L, and F512L relative to SEQ ID NO: 1 comprises the sequence of SEQ ID NO: 77; the sesquiterpene synthase that comprises the amino acid substitutions T72I, N289H, 1291V, V292F, G293Q, T295V, K404W, F406L, T494E, and F512L relative to SEQ ID NO: 1 comprises the sequence of SEQ ID NO: 79; the sesquiterpene synthase that comprises the amino acid substitutions T721, N289H, V292F, T295V, K404W, F406L, T494E, and F512L relative to SEQ ID NO: 1 comprises the sequence of SEQ ID NO: 88; the sesquiterpene synthase that comprises the amino acid substitutions T721, N289H, V292F, G293I, T295V, F406L, 1407G, Y442L, 1507W, and F512L relative to SEQ ID NO: 1 comprises the sequence of SEQ ID NO: 86; the sesquiterpene synthase that comprises the amino acid substitutions P124S, V292F, T295V, K404W, F406L, 1507W, and F512L relative to SEQ ID NO: 1 comprises the sequence of SEQ ID NO: 85; the sesquiterpene synthase that comprises the amino acid substitutions N289H, V292F, T295V, K404W, F406L, Y442L, and F512L relative to SEQ ID NO: 1 comprises the sequence of SEQ ID NO: 87; the sesquiterpene synthase that comprises the amino acid substitutions N289H, F406L, F512L, and T295V relative to SEQ ID NO: 1 comprises the sequence of SEQ ID NO: 89; the sesquiterpene synthase that comprises the amino acid substitutions N289H, F406L, F512L, T295V, and G274S relative to SEQ ID NO: 1 comprises the sequence of SEQ ID NO: 90; the sesquiterpene synthase that comprises the amino acid substitutions N289H, F406L, F512L, T295V, and V201I relative to SEQ ID NO: 1 comprises the sequence of SEQ ID NO: 91; the sesquiterpene synthase that comprises the amino acid substitutions N289H, F406L, F512L, T295V, and 1398C relative to SEQ ID NO: 1 comprises the sequence of SEQ ID NO: 92; the sesquiterpene synthase that comprises the amino acid substitutions T721, N289H, R290K, 1291V, V292F, G293Q, T295V, K404W, F406L, Y442L, T494E, and F512L relative to SEQ ID NO: 1 comprises the sequence of SEQ ID NO: 110; the sesquiterpene synthase that comprises the amino acid substitutions L44V, T721, N289H, 1291V, V292F, G293Q, T295V, K404W, F406L, Y442L, and F512L relative to SEQ ID NO: 1 comprises the sequence of SEQ ID NO: 111; the sesquiterpene synthase that comprises the amino acid substitutions L44V, T72I, T86E, G118Q, T134D, W147L, Q212F, Q217E, S224L, F252L, P255A, N289H, R290K, 1291V, V292F G293Q, T295V, D346E, Y390F, K404W, F406L, L419S, Y442L, T458V, R467K, F512L, and R542V relative to SEQ ID NO: 1 comprises the sequence of SEQ ID NO: 112; the sesquiterpene synthase that comprises the amino acid substitutions T23D, L44V, T72I, N289H, 1291V, V292F, G293Q, T295V, K404W, F406L, Y442L, and F512L relative to SEQ ID NO: 1 comprises the sequence of SEQ ID NO: 113; the sesquiterpene synthase that comprises the amino acid substitutions V201S, N289H, 1291V, V292F, G293N, T295V, Y381W, K404W, F406L, T494E, and F512L relative to SEQ ID NO: 1 comprises the sequence of SEQ ID NO: 114; the sesquiterpene synthase that comprises the amino acid substitutions V201S, V292C, T295I, and D444N relative to SEQ ID NO: 1 comprises the sequence of SEQ ID NO: 115; the sesquiterpene synthase that comprises the amino acid substitutions V201S, V292C, T2951, D444N, and L516V relative to SEQ ID NO: 1 comprises the sequence of SEQ ID NO: 116; the sesquiterpene synthase that comprises the amino acid substitutions L44V, T72I, V201S, V292C, T2951, D444N, and L516V relative to SEQ ID NO: 1 comprises the sequence of SEQ ID NO: 117; the sesquiterpene synthase that comprises the amino acid substitutions L44V, T72I, V201S, V292C, T2951, D444N, and L516A relative to SEQ ID NO: 1 comprises the sequence of SEQ ID NO: 118; the sesquiterpene synthase that comprises the amino acid substitutions L44V, T72I, V201S, V292C, T2951, 1443W, and D444N relative to SEQ ID NO: 1 comprises the sequence of SEQ ID NO: 119; the sesquiterpene synthase that comprises the amino acid substitutions L44V, T72I, V201S, V292C, T295I, D444N, and M519L relative to SEQ ID NO: 1 comprises the sequence of SEQ ID NO: 120; the sesquiterpene synthase that comprises the amino acid substitutions W147V, C188Q, V201S, S224L, V292C, T295I, and D444N relative to SEQ ID NO: 1 comprises the sequence of SEQ ID NO: 121; the sesquiterpene synthase that comprises the amino acid substitutions L44V, T72I, V111L, V201S, S224L, V292C, T2951, 1443T, D444N, T448G, and M519L relative to SEQ ID NO: 1 comprises the sequence of SEQ ID NO: 122; the sesquiterpene synthase that comprises the amino acid substitutions L44V, T72I, V11IL, V201S, V292C, T295I, V433I, 1443T, D444N, T448G, D499N, and L516V relative to SEQ ID NO: 1 comprises the sequence of SEQ ID NO: 123; the sesquiterpene synthase that comprises the amino acid substitutions L44V, T72I, V201S, V292C, T295I, D444N, L516V, and M519L relative to SEQ ID NO: 1 comprises the sequence of SEQ ID NO: 124; the sesquiterpene synthase that comprises the amino acid substitutions L44V, T72I, V201S, V292C, T295I, D444N, V515M, and L516V relative to SEQ ID NO: 1 comprises the sequence of SEQ ID NO: 125; the sesquiterpene synthase that comprises the amino acid substitutions V11IL, V201S, S224L, V292C, T2951, 1443T, D444N, and L516V relative to SEQ ID NO: 1 comprises the sequence of SEQ ID NO: 126; the sesquiterpene synthase that comprises the amino acid substitutions V201S, V292C, T295I, D444N, and L516V relative to SEQ ID NO: 1 comprises the sequence of SEQ ID NO: 127; the sesquiterpene synthase that comprises the amino acid substitutions V201S, V292C, T2951, D444N, T458L, and L516A relative to SEQ ID NO: 1 comprises the sequence of SEQ ID NO: 128; the sesquiterpene synthase that comprises the amino acid substitutions V201S, V292C, T2951, 1443T, D444N, and L516V relative to SEQ ID NO: 1 comprises the sequence of SEQ ID NO: 129; the sesquiterpene synthase that comprises the amino acid substitutions V201S, V292C, T295I, V433I, D444N, and L516V relative to SEQ ID NO: 1 comprises the sequence of SEQ ID NO: 130; or the sesquiterpene synthase that comprises the amino acid substitutions V201S, V292C, T295I, V433L, D444N, and L516V relative to SEQ ID NO: 1 comprises the sequence of SEQ ID NO: 131.

In some embodiments, a sesquiterpene synthase further comprises one or more amino acid substitutions relative to SEQ ID NO: 1, the one or more amino acid substitutions being at one or more positions corresponding to position 294, 296, 297, 403, 444, 515, and/or 525 in SEQ ID NO: 1. In some embodiments, the sesquiterpene synthase comprises:
an L, C, Y, V, A, or S residue at a position corresponding to position 294 in SEQ ID NO: 1;
an L, A, proline (P), Y, N, F or R residue at a position corresponding to position 296 in SEQ ID NO: 1;
an E, Y, I, lysine (K), M, or H residue at a position corresponding to position 297 in SEQ ID NO: 1;
an M, Q, N, S, T, A, E, H, C, or V residue at a position corresponding to position 403 in SEQ ID NO: 1;
an A or N residue at a position corresponding to position 444 in SEQ ID NO: 1; an H, A, E, or Q residue at a position corresponding to position 515 in SEQ ID NO: 1;
an H, C, L, or N residue at a position corresponding to position 525 in SEQ ID NO: 1; or any combination thereof.

In some embodiments, a sesquiterpene synthase does not comprise:
a Q, C, V, F, A, I, H, G, W, or Y residue at a position corresponding to position 289 in SEQ ID NO: 1;
a V, M, or A residue at a position corresponding to position 291 in SEQ ID NO: 1;
an I, M, S, L, G, or N residue at a position corresponding to position 292 in SEQ ID NO: 1;
an I, Q, S, N, or E residue at a position corresponding to position 293 in SEQ ID NO: 1;
a L, V, A, or S residue at a position corresponding to position 295 in SEQ ID NO: 1;
a K or Q residue at a position corresponding to position 296 in SEQ ID NO: 1;
a T or A residue at a position corresponding to position 297 in SEQ ID NO: 1;
a P, F, I, L, G, or D residue at a position corresponding to position 403 in SEQ ID NO: 1;
a G, S, A, I, Y, M, H, V, Q, or C residue at a position corresponding to position 406 in SEQ ID NO: 1;
an H residue at a position corresponding to position 444 in SEQ ID NO: 1;
an M, C, F, G, N, S, or I residue at a position corresponding to position 515 in SEQ ID NO: 1; or any combination thereof.

Table I provides examples of amino acid substitutions in SEQ ID NO: 1 associated 5 with the disclosure.

TABLE 1
Representative amino acid substitutions in Delta-guaiene
synthase 2 protein from A. crassna (SEQ ID NO: 1)

| Position | | | | | | | | | | | |
|---|---|---|---|---|---|---|---|---|---|---|---|
| in SEQ | | | | | | | | | | | |
| ID NO: 1 | Amino Acid Substitutions Relative to SEQ ID NO: 1 | | | | | | | | | | |
| 23 | D | | | | | | | | | | |
| 44 | V | | | | | | | | | | |
| 72 | I | | | | | | | | | | |
| 86 | E | | | | | | | | | | |
| 111 | L | | | | | | | | | | |
| 118 | Q | | | | | | | | | | |
| 122 | N | | | | | | | | | | |
| 124 | S | | | | | | | | | | |
| 134 | D | | | | | | | | | | |
| 147 | V | L | | | | | | | | | |
| 153 | S | | | | | | | | | | |
| 188 | Q | | | | | | | | | | |
| 191 | N | | | | | | | | | | |
| 201 | I | S | | | | | | | | | |
| 205 | E | | | | | | | | | | |
| 212 | F | | | | | | | | | | |
| 217 | E | | | | | | | | | | |
| 224 | L | | | | | | | | | | |
| 252 | L | | | | | | | | | | |
| 255 | A | | | | | | | | | | |
| 274 | S | | | | | | | | | | |
| 275 | G | | | | | | | | | | |
| 289 | L | T | S | D | M | H | K | | | | |
| 290 | K | H | | | | | | | | | |
| 291 | F | L | T | C | V | | | | | | |
| 292 | A | Q | C | Y | H | E | F | T | W | M | |
| 293 | L | V | T | Y | C | F | W | M | Q | I | N |
| 294 | L | C | Y | V | A | S | | | | | |
| 295 | E | D | N | W | G | I | V | | | | |
| 296 | L | A | P | Y | N | R | F | | | | |
| 297 | E | Y | I | K | M | H | | | | | |
| 301 | S | | | | | | | | | | |
| 346 | E | | | | | | | | | | |
| 368 | C | | | | | | | | | | |
| 381 | W | | | | | | | | | | |
| 390 | F | | | | | | | | | | |
| 398 | C | | | | | | | | | | |
| 403 | M | Q | N | V | C | H | E | A | T | S | |
| 404 | W | | | | | | | | | | |
| 406 | L | T | W | N | | | | | | | |
| 407 | G | | | | | | | | | | |
| 419 | S | | | | | | | | | | |
| 433 | I | L | | | | | | | | | |
| 442 | L | | | | | | | | | | |
| 443 | W | T | | | | | | | | | |
| 444 | A | N | | | | | | | | | |
| 448 | G | | | | | | | | | | |
| 458 | L | V | | | | | | | | | |
| 467 | K | | | | | | | | | | |
| 480 | I | | | | | | | | | | |
| 494 | E | | | | | | | | | | |
| 499 | N | | | | | | | | | | |
| 507 | W | | | | | | | | | | |
| 509 | A | | | | | | | | | | |
| 512 | L | | | | | | | | | | |
| 515 | H | A | E | Q | M | | | | | | |
| 516 | A | V | | | | | | | | | |
| 519 | L | | | | | | | | | | |
| 525 | H | C | L | N | | | | | | | |
| 526 | F | | | | | | | | | | |
| 533 | N | | | | | | | | | | |
| 542 | V | | | | | | | | | | |

In some embodiments, a sesquiterpene synthase comprises a sequence that is at least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to any one of SEQ ID NOs: 3-39, 77-92 or 110-131 or a conservatively substituted variant thereof. In certain embodiments, a sesquiterpene synthase comprises the sequence of any one of SEQ ID NOs: 3-39, 77-92 or 110-131 or a conservatively substituted version thereof. In certain embodiments a sesquiterpene synthase consists of or consists essentially of the sequence of any one of SEQ ID NOs: 3-39, 77-92 or 110-131 or a conservatively substituted variant thereof.

In some embodiments, a sesquiterpene synthase associated with the disclosure, or a host cell expressing a sesquiterpene synthase associated with the disclosure, produces one or more sesquiterpenes, including alpha-guaiene and/or delta-guaiene. In some embodiments, a sesquiterpene synthase associated with the disclosure, or a host cell expressing a sesquiterpene synthase associated with the disclosure, produces at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% alpha-guaiene as a percentage of total sesquiterpene products as determined by GC, or produces 100% alpha-guaiene as a percentage of total sesquiterpene products as determined by GC. In some embodiments, a sesquiterpene synthase associated with the disclosure, or a host cell expressing a sesquiterpene synthase associated with the disclosure, produces at least the preceding recited percentages more alpha-guaiene than that produced by the wild type enzyme of SEQ ID NO: 1 or a host cell expressing the wild type enzyme as determined by GC. In some embodiments, a sesquiterpene synthase associated with the disclosure, or a host cell expressing a sesquiterpene synthase associated with the disclosure, produce at least 50% alpha-guaiene as a percentage of total sesquiterpene products as determined by GC or at least 50% more alpha-guaiene than that produced by the wild type enzyme of SEQ ID NO: 1 or a host cell expressing the wild type enzyme as determined by GC.

In some embodiments, a sesquiterpene synthase associated with the disclosure, or a host cell expressing a sesquiterpene synthase associated with the disclosure, produces about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69% or 70% delta-guaiene as a percentage of total sesquiterpene products as determined by GC, or produces 100% delta-guaiene as a percentage of total sesquiterpene products as determined by GC.

In some embodiments, a sesquiterpene synthase associated with the disclosure, or a host cell expressing a sesquiterpene synthase associated with the disclosure, produces more alpha-guaiene than delta-guaiene. For example, in some embodiments, a sesquiterpene synthase associated with the disclosure, or a host cell expressing a sesquiterpene synthase associated with the disclosure, produces at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300%, 350%, 400%, 450%, or 500% more alpha-guaiene than delta-guaiene as determined by GC. In some embodiments, a sesquiterpene synthase associated with the disclosure, or a host cell expressing a sesquiterpene synthase associated with the disclosure, produces at least 0.5, 1, 2, 3, 4, 5, 6, 7, 8, 9, or 10-fold more alpha-guaiene than delta-guaiene as determined by GC.

In some embodiments, a sesquiterpene synthase associated with the disclosure, or a host cell expressing a sesquiterpene synthase associated with the disclosure, produces about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69% or 70% aciphyllene as a percentage of total sesquiterpene products as determined by GC, or produces 100% aciphyllene as a percentage of total sesquiterpene products as determined by GC.

In some embodiments, a sesquiterpene synthase associated with the disclosure, or a host cell expressing a sesquiterpene synthase associated with the disclosure, produces more alpha-guaiene than aciphyllene. For example, in some embodiments, a sesquiterpene synthase associated with the disclosure, or a host cell expressing a sesquiterpene synthase associated with the disclosure, produces at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300%, 350%, 400%, 450%, or 500% more alpha-guaiene than aciphyllene as determined by GC. In some embodiments, a sesquiterpene synthase associated with the disclosure, or a host cell expressing a sesquiterpene synthase associated with the disclosure, produces at least 0.5, 1, 2, 3, 4, 5, 6, 7, 8, 9, or 10-fold more alpha-guaiene than aciphyllene as determined by GC.

In some embodiments, a sesquiterpene synthase associated with the disclosure, or a host cell expressing a sesquiterpene synthase associated with the disclosure, produces more alpha-guaiene than that produced by the wild type enzyme of SEQ ID NO: 1 or a host cell expressing the wild type enzyme. For example, in some embodiments, a sesquiterpene synthase associated with the disclosure, or a host cell expressing a sesquiterpene synthase associated with the disclosure, produces at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300%, 350%, 400%, 450%, or 500% more alpha-guaiene than that produced by the wild type enzyme of SEQ ID NO: 1 or a host cell expressing the wild type enzyme as determined by GC. In some embodiments, a sesquiterpene synthase associated with the disclosure, or a host cell expressing a sesquiterpene synthase associated with the disclosure, produces at least 0.5, 1, 2, 3, 4, 5, 6, 7, 8, 9, or 10-fold more alpha-guaiene than that produced by the wild type enzyme of SEQ ID NO: 1 or a host cell expressing the wild type enzyme as determined by GC.

In some embodiments, a sesquiterpene synthase associated with the disclosure, or a host cell expressing a sesquiterpene synthase associated with the disclosure, produces a higher ratio of alpha-guaiene to delta-guaiene than that produced by a sesquiterpene synthase comprising the sequence of SEQ ID NO: 1 or a host cell expressing a sesquiterpene synthase comprising the sequence of SEQ ID NO: 1. In some embodiments, a sesquiterpene synthase associated with the disclosure, or a host cell expressing a sesquiterpene synthase associated with the disclosure, produces more alpha-guaiene as a percentage of total sesquiterpene products than that produced by a sesquiterpene synthase comprising the sequence of SEQ ID NO: 1 or a host cell expressing a sesquiterpene synthase comprising the sequence of SEQ ID NO: 1. In some embodiments, sesquiterpene synthase associated with the disclosure, or a host cell expressing a sesquiterpene synthase associated with the disclosure, produces less delta-guaiene as a percentage of total sesquiterpene products than that produced a sesquiterpene synthase comprising the sequence of SEQ ID NO: 1 or a host cell expressing a sesquiterpene synthase comprising the sequence of SEQ ID NO: 1. In some embodiments, a sesquiterpene synthase associated with the disclosure, or a host cell expressing a sesquiterpene synthase associated with the disclosure, produces more alpha-guaiene than that produced by a sesquiterpene synthase comprising the sequence of SEQ ID NO: 1 or a host cell expressing a sesquiterpene synthase comprising the sequence of SEQ ID NO: 1. In some embodiments, a sesquiterpene synthase associated with the disclosure, or a host cell expressing a sesquiterpene synthase associated with the disclosure, produces more aciphyllene than that produced by a sesquiterpene synthase comprising the sequence of SEQ ID NO: 1 or a host cell expressing a sesquiterpene synthase comprising the sequence of SEQ ID NO: 1. In some embodiments, a sesquiterpene synthase associated with the disclosure, or a host cell expressing a sesquiterpene synthase associated with the disclosure, produces more alpha-guaiene and aciphyllene than that produced by a sesquiterpene synthase comprising the sequence of SEQ ID NO: 1 or a host cell expressing a sesquiterpene synthase comprising the sequence of SEQ ID NO: 1.

For example, in some embodiments, a sesquiterpene synthase associated with the disclosure, or a host cell expressing a sesquiterpene synthase associated with the disclosure, produces at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300%, 350%, 400%, 450%, or 500% more alpha-guaiene as a percentage of total sesquiterpene products than that produced by a sesquiterpene synthase comprising the sequence of SEQ ID NO: 1 or a host cell expressing a sesquiterpene synthase comprising the sequence of SEQ ID NO: 1. In some embodiments, a sesquiterpene synthase associated with the disclosure, or a host cell expressing a sesquiterpene synthase associated with the disclosure, produces at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300%, 350%, 400%, 450%, or 500% less delta-guaiene as a percentage of total sesquiterpene products than that produced by a sesquiterpene synthase comprising the sequence of SEQ ID NO: 1 or a host cell expressing a sesquiterpene synthase comprising the sequence of SEQ ID NO: 1. In some embodiments, a sesquiterpene synthase associated with the disclosure, or a host cell expressing a sesquiterpene synthase associated with the disclosure, produces at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300%, 350%, 400%, 450%, or 500% more alpha-guaiene as a percentage of total sesquiterpene products than that produced by a sesquiterpene synthase comprising the sequence of SEQ ID NO: 1 or a host cell expressing a sesquiterpene synthase comprising the sequence of SEQ ID NO: 1.

It should be appreciated that in some embodiments, total sesquiterpene products within a sample may be measured and the amount of alpha-guaiene or delta-guaiene relative to the amount of total sesquiterpene products in the sample may be calculated. It should be appreciated that in some embodiments, total sesquiterpene products within a sample may be measured and the amount of alpha-guaiene or aciphyllene relative to the amount of total sesquiterpene products in the sample may be calculated. In other embodiments, less than all of the sesquiterpene products in a sample may be measured. For example, in some embodiments, only the amounts of alpha-guaiene and delta-guaiene may be measured in a sample. In some embodiments, only the amounts of alpha-guaiene and aciphyllene may be measured in a sample. In such embodiments the amount of alpha-guaiene relative to the combined amount of alpha-guaiene and delta-guaiene in the sample, or the amount of alpha-guaiene relative to the amount of delta-guaiene in the sample, may be calculated.

In some embodiments the amount of alpha-guaiene produced by a host cell, cell culture, or enzyme associated with the disclosure is at least 1 g/L. In some embodiments the amount of alpha-guaiene produced by a host cell, cell culture, or enzyme associated with the disclosure is at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5 or 5.0 g/L.

In some embodiments the amount of aciphyllene produced by a host cell, cell culture, or enzyme associated with the disclosure is at least 1 g/L. In some embodiments the amount of alpha-guaiene produced by a host cell, cell culture, or enzyme associated with the disclosure is at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5 or 5.0 g/L.

The mass of sesquiterpenes can be determined by any method known in the art. In some embodiments, the mass of sesquiterpenes is determined by GCMS.

In some embodiments, an amino acid substitution is introduced in a residue that is at or near the active site of the sesquiterpene synthase. Mutations at or near the active site may alter the structural conformation or the catalytic activity of the enzyme and bias the enzyme to produce one product over another. In some embodiments, the amino acid substitution results in a hydrophilic (polar) amino acid residue at or near the active site. As used in this disclosure, a “hydrophilic residue” refers to a positively charged amino acid that attracts water molecules. In some embodiments, the amino acid substitution results in a hydrophobic (nonpolar) amino acid residue at or near the active site. As used in this disclosure, a “hydrophobic residue” refers to a non-polar amino acid that repels water molecules. In some embodiments, the amino acid substitution results in an introduction of an aromatic amino acid residue at or near the active site that may help coordinate cation-dipole interactions to affect reaction specificity. Examples of an aromatic amino acid residue include tyrosine, tryptophane, and phenylalanine.

Without wishing to be bound by any theory, the shape of the active site pocket of the sesquiterpene synthase may influence the ratio of different products produced by the sesquiterpene synthase. In the reaction catalyzed by the Delta-guaiene synthase 2 protein from A. crassna (UniProt Accession No. D0VMR7; SEQ ID NO: 1), the final step involves a proton abstraction from either the pro-delta carbon, leading to delta-guaiene as the product, or the pro-alpha carbon, leading to alpha-guaiene as the product. In some embodiments, a variant sesquiterpene synthase may produce increased amounts of alpha-guaiene at least in part because it alters the substrate binding mode of the sesquiterpene synthase to allow easier access to the pro-alpha carbon from the catalytic residue Tyr-520.

In some embodiments, a sesquiterpene synthase associated with the disclosure comprises: (a) one or more amino acid substitutions in a first region of the active site relative to SEQ ID NO: 1, wherein the one or more amino acid substitutions in the first region are at positions that correspond to positions 295, 291, 406, 512, and/or 519 of SEQ ID NO: 1, and the one or more amino acid substitutions in the first region each comprises a residue with a smaller side chain than that of the amino acid at positions 295, 291, 406, 512, and/or 519 in SEQ ID NO: 1; (b) an amino acid substitution in a second region of the active site relative to SEQ ID NO: 1, wherein the amino acid substitution in the second region corresponds to position 292 of SEQ ID NO: 1, and wherein the amino acid substitution in the second region comprises a larger side chain than that of the amino acid at position 292 in SEQ ID NO: 1; or any combination thereof.

In some embodiments, the side chain in (a) is hydrophobic. In some embodiments, an amino acid residue having a smaller side chain in (a) is selected from the group consisting of alanine (A), serine(S), threonine (T), and valine (V). In some embodiments, an amino acid residue having a larger side chain in (b) is selected from the group consisting of arginine (R), phenylalanine (F), tyrosine (Y), and tryptophan (W).

In some embodiments, a sesquiterpene synthase associated with the disclosure comprises:
a V residue at a position corresponding to position 295 in SEQ ID NO: 1;
a C residue at a position corresponding to position 291 in SEQ ID NO: 1;
an L residue at a position corresponding to position 406 in SEQ ID NO: 1;
an L residue at a position corresponding to position 512 in SEQ ID NO: 1;
a F residue at a position corresponding to position 292 in SEQ ID NO: 1; or any combination thereof.

### Variants

Aspects of the disclosure relate to polynucleotides encoding any of the recombinant polypeptides, such as sesquiterpene synthases, associated with the disclosure. Variants of polynucleotide or polypeptide sequences described in this application are also encompassed by the present disclosure. As used in this disclosure, a “variant” polynucleotide refers to a polynucleotide for which the nucleic acid sequence differs from the nucleic acid sequence of a reference polynucleotide by one or more changes in the nucleic acid sequence. As used in this disclosure, a “variant” polypeptide refers to a polypeptide for which the amino acid sequence differs from the amino acid sequence of a reference polypeptide by one or more changes in the amino acid sequence.

A variant polynucleotide or polypeptide can be constructed synthetically. Typically, the polynucleotide or polypeptide from which a variant is derived is a wild-type polynucleotide, a wild-type polypeptide, or a wild-type polynucleotide or polypeptide domain. However, the variants usable in the present disclosure may also be derived from homologs, orthologs, or paralogs of a wild-type polynucleotide, a wild-type polypeptide, or a wild-type polynucleotide or polypeptide domain, or from synthetic polynucleotides or polypeptides. The changes in the nucleic acid and/or amino acid sequences may include substitutions, insertions, deletions, N-terminal truncations, (′-terminal truncations, N-terminal additions, (′-terminal additions, or any combination of these changes, which may occur at one or multiple positions.

A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, while in other embodiments, sequence identity is determined over a region of a sequence.

Identity can also refer to the degree of sequence relatedness between two sequences as determined by the number of matches between strings of two or more residues (e.g., nucleic acid or amino acid residues). Identity measures the percent of identical matches between two or more sequences with gap alignments (if any) addressed by a particular mathematical model, algorithms, or computer program.

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al., Nucleic Acids Res. 25 (17): 3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotides and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

Functional variants of sesquiterpene synthases and any other proteins disclosed in this application are also encompassed by the present disclosure. As used in this disclosure, a functional variant of a sesquiterpene synthase refers to a sesquiterpene synthase that has a different sequence than the sequence of a reference sesquiterpene synthase but that maintains, partially or fully, at least one activity of the reference sesquiterpene synthase. In some embodiments, a functional variant of a sesquiterpene synthase enhances one or more activities of a reference sesquiterpene synthase. For example, a functional variant may bind one or more of the same substrates (e.g., farnesyl diphosphate, or precursors thereof) or produce one or more of the same products (e.g., alpha-guaiene).

Variant sequences, including functional variants, may be homologous sequences. Homologous sequences include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution. As used in this disclosure, a functional homolog of a reference sesquiterpene synthase maintains, partially or fully, at least one activity of the reference sesquiterpene synthase. In some embodiments, a functional homolog of a sesquiterpene synthase enhances one or more activities of a reference sesquiterpene synthase. For example, a functional homolog may bind one or more of the same substrates (e.g., farnesyl diphosphate, or precursors thereof) or produce one or more of the same products (e.g., alpha-guaiene).

Functional variants may be variants of naturally occurring sequences. Functional variants can also be created via site-directed mutagenesis of the coding sequence for a polypeptide, or by combining domains from the coding sequences for different naturally-occurring polypeptides (“domain swapping”). Techniques for modifying genes encoding functional variants described in this disclosure are known in the art and include, inter alia, directed evolution techniques, site-directed mutagenesis techniques and random mutagenesis techniques, and can be useful, for example, to increase specific activity of a polypeptide, alter substrate specificity, alter expression levels, alter subcellular location, or modify polypeptide:polypeptide interactions in a desired manner.

Variants and homologs can be identified by analysis of polynucleotide and polypeptide sequence alignments. For example, performing a query on a database of polynucleotide or polypeptide sequences can identify variants and homologs of polynucleotide sequences encoding derivative polypeptides and the like.

Hybridization can also be used to identify functional variants or functional homologs and/or as a measure of homology between two polynucleotide sequences. A polynucleotide sequence encoding any of the polypeptides disclosed in this application, or a portion thereof, can be used as a hybridization probe according to standard hybridization techniques. The hybridization of a probe to DNA or RNA from a test source (e.g., a mammalian cell) is an indication of the presence of the relevant DNA or RNA in the test source. Hybridization conditions are known to those skilled in the art and can be found in, e.g., Current Protocols in Molecular Biology, John Wiley & Sons, N.Y., 6.3.1-6.3.6, 1991. In some embodiments, moderate hybridization conditions include hybridization in 2× sodium chloride/sodium citrate (SSC) at 30° C. followed by a wash in 1×SSC, 0.1% SDS at 50° C. In some embodiments, highly stringent conditions include hybridization in 6× sodium chloride/sodium citrate (SSC) at 45° C. followed by a wash in 0.2×SSC, 0.1% SDS at 65° C.

Sequence analysis to identify functional variants or functional homologs can also involve BLAST, Reciprocal BLAST, or PSI-BLAST analysis of non-redundant databases using a relevant amino acid sequence as the reference sequence. An amino acid sequence is, in some instances, deduced from a polynucleotide sequence. In some embodiments, polypeptides that have greater than 40% sequence identity may be identified as candidates for further evaluation for suitability for use according to the disclosure. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have, e.g., conserved functional domains.

In some embodiments, a polypeptide variant (e.g., sesquiterpene synthase variant or variant of any protein associated with the disclosure) comprises a domain that shares a secondary structure (e.g., alpha helix, beta sheet) with a reference polypeptide (e.g., a reference sesquiterpene synthase, or any protein associated with the disclosure). In some embodiments, a polypeptide variant (e.g., sesquiterpene synthase variant or variant of any protein associated with the disclosure) shares a tertiary structure with a reference polypeptide (e.g., a reference sesquiterpene synthase, or any protein associated with the disclosure). In some embodiments, a reference polypeptide is a sesquiterpene synthase comprising the sequence of SEQ ID NO: 1. As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82:488-492, 1985), by chemical synthesis of a gene encoding a polypeptide, by gene editing tools, or by insertions, such as insertion of a tag (e.g., a HIS tag or a GFP tag). Mutations can include, for example, substitutions, deletions, additions, insertions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January;29 (1): 18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce a protein with different functional characteristics (e.g., increased or decreased enzymatic activity, different substrate specificity, or different product specificity). See, e.g., Yu and Lutz, Trends Biotechnol. 2011 January;29 (1): 18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1;21 (7): 932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants or functional homologs may be identified using any method known in the art. For example, the algorithm of Karlin and Altschul Proc. Natl. Acad. Sci. USA 87:2264-68, 1990 described above may be used to identify homologous proteins with known functions.

Putative functional variants or functional homologs may also be identified by searching for polypeptides with functionally annotated domains. Databases including Pfam (Sonnhammer et al., Proteins. 1997 July;28 (3): 405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol. See, e.g., Stormo et al., Nucleic Acids Res. 1982 May 11;10 (9): 2997-3011.

PSSM may be paired with calculation of a Rosetta energy function, which determines the difference between the wild-type and the single-point mutant. Without being bound by a particular theory, potentially stabilizing mutations are desirable for protein engineering (e.g., production of functional homologs). In some embodiments, a potentially stabilizing mutation has a ΔΔG_{calc} value of less than-0.1 (e.g., less than-0.2, less than-0.3, less than-0.35, less than-0.4, less than-0.45, less than-0.5, less than-0.55, less than-0.6, less than-0.65, less than-0.7, less than-0.75, less than-0.8, less than-0.85, less than-0.9, less than-0.95, or less than-1.0) Rosetta energy units (R.e.u.). See, e.g., Goldenzweig et al., Mol Cell. 2016 Jul. 21;63 (2): 337-346. doi: 10.1016/j.molcel.2016.06.012.

In some embodiments, a sesquiterpene synthase coding sequence or coding sequence of any protein associated with the disclosure comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference coding sequence. In some embodiments, the sesquiterpene synthase coding sequence or coding sequence of any protein associated with the disclosure comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a sesquiterpene synthase sequence or other recombinant protein sequence associated with the disclosure alter the amino acid sequence of the polypeptide relative to the amino acid sequence of a reference polypeptide. In some embodiments, the one or more mutations alter the amino acid sequence of the recombinant polypeptide relative to the amino acid sequence of a reference polypeptide and alter (enhance or reduce) an activity of the polypeptide relative to the reference polypeptide.

Assays for determining and quantifying enzyme and/or enzyme variant activity are described herein and are known in the art. By way of example, enzyme and/or enzyme variant activity can be determined by incubating a purified enzyme or enzyme variant or extracts from host cells or a complete recombinant host organism that has produced the enzyme or enzyme variant with an appropriate substrate under appropriate conditions and carrying out an analysis of the reaction products (e.g., by gas chromatography (GC) or HPLC analysis). Further details on enzyme and/or enzyme variant activity assays and analysis of the reaction products are provided in the Examples. These assays include producing enzyme variants in recombinant host cells.

The activity, including specific activity, of any of the enzymes described in this application may be measured using methods known in the art. As a non-limiting example, an enzyme's activity may be determined by measuring its substrate specificity, product(s) produced, the concentration of product(s) produced, or any combination thereof.

As used in this disclosure, the term “activity” means the ability of an enzyme to react with a substrate to provide a target product. The activity of an enzyme can be determined in an activity test via measuring the increase of one or more target products, the decrease of one or more substrates (or starting materials) or via measuring a combination of these parameters as a function of time. As used in this application, “specific activity” of an enzyme refers to the amount (e.g., concentration) of a particular product produced for a given amount (e.g., concentration) of the enzyme per unit time.

A “biological activity” as used in this disclosure, refers to any activity a polypeptide may exhibit, including without limitation: enzymatic activity; binding activity to another compound (e.g., binding to another polypeptide, in particular binding to a receptor, or binding to a nucleic acid); inhibitory activity (e.g., enzyme inhibitory activity); activating activity (e.g., enzyme-activating activity); or toxic effects. In some embodiments, a functional variant polypeptide exhibits the relevant activity to a degree of at least 10% of the activity of the parent or reference polypeptide.

In some embodiments, a functional variant of an enzyme associated with the present disclosure produces a better yield than a reference or parent enzyme (e.g., a wild-type enzyme or a reference enzyme variant). As used in this disclosure, the term “yield” refers to the gram of recoverable product per gram of feedstock (which can be calculated as a percent molar conversion rate).

In some embodiments, a functional variant of an enzyme associated with the present disclosure exhibits modified (e.g., increased) productivity relative to a reference or parent enzyme (e.g., a wild-type enzyme or a reference enzyme variant).

As used in this disclosure, “productivity” of a variant sesquiterpene synthase refers to the fold increase in production of a desired product by the variant sesquiterpene synthase relative to the production of the desired product by a reference or parent enzyme (e.g., a wild-type enzyme or a reference enzyme variant). For example, when the desired product is alpha-guaiene, then productivity of a variant sesquiterpene synthase refers to the fold increase in production of alpha-guaiene by the variant sesquiterpene synthase relative to the production of alpha-guaiene by a reference or parent enzyme (e.g., a wild-type enzyme or a reference enzyme variant).

In some embodiments, a functional variant of an enzyme associated with the present disclosure exhibits a better target yield than a reference or parent enzyme The term “target yield” refers to the gram of recoverable product per gram of feedstock (which can be calculated as a percent molar conversion rate).

In some embodiments, a functional variant of an enzyme associated with the present disclosure exhibits a modified (e.g., increased) target productivity relative to a reference or parent enzyme. The term “target productivity” refers to the amount of recoverable target product in grams per liter of fermentation capacity per hour of bioconversion time (i.e., time after the substrate was added).

In some embodiments, a functional variant of an enzyme associated with the present disclosure exhibits a modified target yield factor relative to a reference or parent enzyme. The term “target yield factor” refers to the ratio between the product concentration obtained and the concentration of the variant/derivative (for example, purified enzyme or an extract from a recombinant host cell expressing the desired enzyme) in culture medium.

In some embodiments, a functional variant of an enzyme associated with the present disclosure exhibits a modified (e.g., increased) fold in enzymatic activity relative to a reference or parent enzyme (e.g., any one of SEQ ID NOs: 1, 3-39, 77-92 or 110-131). In some embodiments, the increase in activity is by at least a factor of: 2, 3, 4, 6, 8, 10, 12, 14, 16, 18, 20, 25, 30, 35, 40, 45, 50, 55, 60, 65, 70, 75, 80, 85, 90, 95, 100 or more than 100.

In some embodiments, a functional variant of an enzyme associated with the present disclosure exhibits a modified (e.g., increased) target productivity relative to a reference or parent enzyme. The term “target productivity” refers to the amount of recoverable target product in grams per liter of fermentation capacity per hour of bioconversion time (i.e., time after the substrate was added).

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

Accordingly, as used in this disclosure, the term “conservative substitution” means an exchange of an amino acid by another amino acid listed within the same group of the six standard amino acid groups shown below.
(1) hydrophobic (non-polar): Met, Ala, Val, Leu, Ile, Gly, Pro, Trp, Phe;
(2) neutral hydrophilic: Cys, Ser, Thr; Asn, Gln, Tyr;
(3) acidic: Asp, Glu;
(4) basic: His, Lys, Arg;
(5) residues that influence chain orientation: Gly, Pro;
(6) aromatic: Trp, Tyr, Phe.

For example, the exchange of Asp by Glu retains one negative charge in the modified polypeptide. In addition, glycine and proline may be substituted for one another based on their ability to disrupt alpha-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe. Given the known genetic code, and recombinant and synthetic DNA techniques, the skilled scientist readily can construct polynucleotide sequences encoding conservatively substituted amino acid variants.

As used herein, “non-conservative substitutions” or “non-conservative amino acid exchanges” are defined as exchanges of an amino acid by another amino acid listed in a different group of the six standard amino acid groups (1) to (6) as shown above. In some embodiments, variants of enzymes associated with the present disclosure are prepared using non-conservative substitutions that alter the biological function of the variants.

For ease of reference, the one-letter amino acid symbols recommended by the IUPAC-IUB Biochemical Nomenclature Commission are indicated as follows. The three letter codes are also provided for reference purposes.

TABLE 2
Amino Acid Symbols

| One Letter Code | Three Letter Code | Amino acid name |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Amino acid alterations such as amino acid substitutions may be introduced using known protocols of recombinant gene technology including PCR, gene cloning, site-directed mutagenesis of cDNA, transfection of host cells, and in-vitro transcription which may be used to introduce such changes to a sequence resulting in a variant/derivative enzyme. Variants containing amino acid alterations can be screened for functional activity. In some instances, an amino acid is characterized by its R group (see, e.g., Table 3). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 3.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 3
Non-limiting examples of conservative amino acid substitutions

| Original | | Conservative Amino |
|---|---|---|
| Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R group | Ala, Ser |
| Gln (Q) | polar uncharged R group | Asn, Asp, Glu |
| Glu (E) | negatively charged R group | Asn, Asp, Gln |
| Gly (G) | nonpolar aliphatic R group | Ala, Ser |
| His (H) | positively charged R group | Arg, Tyr, Trp |
| Ile (I) | nonpolar aliphatic R group | Leu, Met, Val |
| Leu (L) | nonpolar aliphatic R group | Ile, Met, Val |
| Lys (K) | positively charged R. group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

In some embodiments of the disclosure, an amino acid at a particular position in a protein may be replaced by an amino acid that has a different molecular weight. For example, in some embodiments, an amino acid at a particular position in a protein may be replaced by a “larger” amino acid, which refers to an amino acid that has a larger molecular weight. In other embodiments, an amino acid at a particular position in a protein may be replaced by a “smaller” amino acid, which refers to an amino acid that has a smaller molecular weight. The amino acids, ranked from smallest to largest based on molecular weight are: G, A, S, P, V, T, C, I, L, N, D, E, K, Q, M, H, F, R, Y, and W.

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide (e.g., sesquiterpene synthase, or any other protein associated with the disclosure).

### Expression of Nucleic Acids in Host Cells

Aspects of the present disclosure relate to the recombinant expression of genes encoding proteins, functional modifications and variants thereof, as well as uses relating thereto. For example, the methods described in this application may be used to produce alpha-guaiene.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system; or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13 (7): 563-567. A heterologous polynucleotide may comprise a wild-type sequence or a mutant sequence as compared with a reference polynucleotide sequence.

A nucleic acid encoding any of the recombinant polypeptides, such as sesquiterpene synthases, or any proteins associated with the disclosure, may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. A vector can contain one or more endonuclease restriction sites that are cut by a restriction endonuclease to insert and ligate a nucleic acid containing a gene described in this application to produce a recombinant vector that is able to replicate in a cell. Vectors can be composed of DNA or RNA. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell, such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker, to identify cells transformed or transfected with the recombinant vector. In some embodiments, the nucleic acid sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined or linked if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, tetracycline, galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters known to one of ordinary skill in the art are also contemplated.

Regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. Vectors may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a host cell is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

In some embodiments, introduction of a polynucleotide, such as a polynucleotide encoding a recombinant polypeptide, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the recombinant polypeptides described in this application, in its genome. Said copies may be inserted into the same locus or into different loci of a recombinant host cell of the disclosure.

In some embodiments, a polynucleotide encoding a sesquiterpene synthase comprises a sequence that is at least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to any one of SEQ ID NOs: 40-76, 93-109 or 133-154. In certain embodiments, a polynucleotide encoding a sesquiterpene synthase comprises any one of SEQ ID NOs: 40-76, 93-109 or 133-154. In certain embodiments a polynucleotide encoding a sesquiterpene synthase consists of or consists essentially of any one of SEQ ID NOs: 40-76, 93-109, 133-154.

### Host Cells

Any of the proteins of the disclosure may be expressed in a host cell. As used in this application, the term “host cell” refers to a cell that can be used to express a polynucleotide, such as a polynucleotide that encodes a protein used in production of alpha-guaiene and precursors thereof.

Any suitable host cell may be used to express any of the recombinant polypeptides, including sesquiterpene synthases, and other proteins disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranaefaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica. In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In some embodiments, the host cell is a Saccharomyces cell, such as a S. cerevisiae cell.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica). The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

In various embodiments, strains that may be used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The term “cell,” as used in this application, may refer to a single cell or a population of cells, such as a population of cells belonging to the same cell line or strain. Use of the singular term “cell” should not be construed to refer explicitly to a single cell rather than a population of cells.

A vector encoding any of the recombinant polypeptides described in this application may be introduced into a suitable host cell using any method known in the art. For example, non-limiting examples of yeast transformation protocols are described in Gietz et al., Yeast transformation can be conducted by the LiAc/SS Carrier DNA/PEG method. Methods Mol Biol. 2006; 313:107-20, which is incorporated by reference in its entirety. Host cells may be cultured under any suitable conditions as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The term “media” refers interchangeably to a culture medium or cultivation medium or fermentation medium. Typically a culture medium comprises components essential or beneficial to the maintenance and/or growth of a cell such as carbon sources or carbon substrate, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids, vitamins, growth promoters, and the like. The term “carbon source” or “carbon substrate” or “source of carbon” according to the present disclosure denotes any source of carbon that can be used by those skilled in the art to support the normal growth of a cell, including hexoses (such as glucose, galactose or lactose), pentoses, monosaccharides, oligosaccharides, disaccharides (such as sucrose, cellobiose or maltose), molasses, starch or its derivatives, cellulose, hemicelluloses and combinations thereof.

The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism, part of a living organism, or purified proteins. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art of bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., alpha-guaiene) may display some differences from the substrate (e.g., farnesyl diphosphate) in terms of solubility, toxicity, cellular accumulation and secretion and in some embodiments can have different fermentation kinetics.

Aspects of the present disclosure provide methods of increasing production of a compound of interest, e.g., alpha-guaiene in a host cell by increasing sesquiterpene synthase activity by introducing one or more mutation(s) described in this disclosure into a sesquiterpene synthase. The methods described in this application encompass production of alpha-guaiene using a host cell, cell lysate or isolated recombinant polypeptides (e.g., sesquiterpene synthase, and any other proteins associated with the disclosure).

Alpha-guaiene produced by any of the recombinant cells disclosed in this application may be identified and extracted using any method known in the art. GC (e.g., GC-MS, GC-FID) is a non-limiting example of a method for identification and/or quantitation of a compound of interest.

### Compositions

Further aspects of the disclosure relate to compositions containing alpha-guaiene. Culturing of host cells associated with the disclosure can result in compositions comprising sesquiterpene products, including alpha-guaiene. In some embodiments, compositions obtained by culturing host cells associated with the disclosure result in compositions in which at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the total sesquiterpene products in the composition is alpha-guaiene.

In some embodiments, compositions obtained by culturing host cells associated with the disclosure result in compositions in which about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% of the total sesquiterpene products in the composition is delta-guaiene.

Compositions associated with the disclosure can further comprise additional components as would be understood by one of ordinary skill in the art. For example, it should be appreciated that in some embodiments, compositions comprising alpha-guaiene can include cell culture fermentation broth or cell culture supernatants. In other embodiments, compositions may include alpha-guaiene in a form that has been purified from cell culture fermentation broth or cell culture supernatants.

The ratio of alpha-guaiene to delta-guaiene may, for example, range from about 60:40 to about 99:1. For example, the weight ratio of alpha-guaiene to delta-guaiene may range from about 65:35 to about 99:1, from about 70:30 to about 99:1, from about 75:25 to about 99:1, from about 80:20 to about 99:1, from about 85:15 to about 99:1, from about 90:10 to about 99:1, or from about 95:5 to about 99:1. For example, the weight ratio of alpha-guaiene to delta-guaiene may range from about 65:35 to about 98:2, from about 70:30 to about 97:3, from about 75:25 to about 96:4, from about 80:20 to about 95:5, or from about 85:15 to about 90:10.

In some embodiments, cells associated with the invention are cultured in the presence of an organic solvent overlay. As used in this disclosure, an organic solvent overlay refers to a layer comprising one or more organic solvents that is added to a cell culture sample. The organic solvent overlay may partially or fully cover the cell culture sample. The use of an organic solvent overlay can assist with reducing or alleviating host cell toxicity caused by increased concentrations of products. In some embodiments, compositions comprising alpha-guaiene further comprise one or more components of an organic solvent overlay (e.g., dodecane).

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### Examples

### Example 1. Identification of Variant Sesquiterpene Synthases that Produce Increased Alpha-Guaiene

This Example describes the identification of variant sesquiterpene synthases capable of producing increased amounts of alpha-guaiene relative to that produced by the parent Delta-guaiene synthase 2 protein from A. crassna (UniProt Accession No. D0VMR7; SEQ ID NO: 1).

A metagenomic library of putative sesquiterpene synthases was sourced from public and private sequence databases. After codon optimization for yeast, sequences were screened for alpha-guaiene production using a high-throughput GC-MS assay. Of the approximately 500 sesquiterpene synthases designed and tested, none made alpha-guaiene in an amount in excess of 15% as determined using extracted ion chromatograms (EIC) m/z fragments for the sesquiterpene synthase products detected via GC-MS utilizing authentic alpha-guaiene standard for identification.

Since the metagenomic screen did not identify any putative sesquiterpene synthases showing increased alpha-guaiene production, a second approach for identifying sesquiterpene synthases capable of producing increased amounts of alpha-guaiene was pursued. This approach involved generating sequence variants of Delta-guaiene synthase 2 protein from A. crassna (UniProt Accession No. D0VMR7; SEQ ID NO: 1).

Variant sesquiterpene synthases were generated by PCR mutagenesis and synthesized in yeast expression vectors flanked by a GAL1 promoter and terminator. Plasmids containing variant sesquiterpene synthase DNA coding sequences were transformed into screening strains for in vivo evaluation of enzymatic performance. The screening strains were engineered to express higher concentrations of farnesyl diphosphate (FPP), which serves as a substrate for the enzymes being screened. Within the screening strains, the sesquiterpene synthases turn over the FPP when galactose is available to the screening strain. The GAL1 promoter is responsive to galactose and induces variant enzyme expression in the presence of galactose. After sufficient exposure to galactose, the strains were harvested and exposed to an organic solvent, which was used to isolate the reaction products. Once isolated, the reaction products were analyzed by GC to determine the relative abundance, proportions, and identity of products in the reaction product mix.

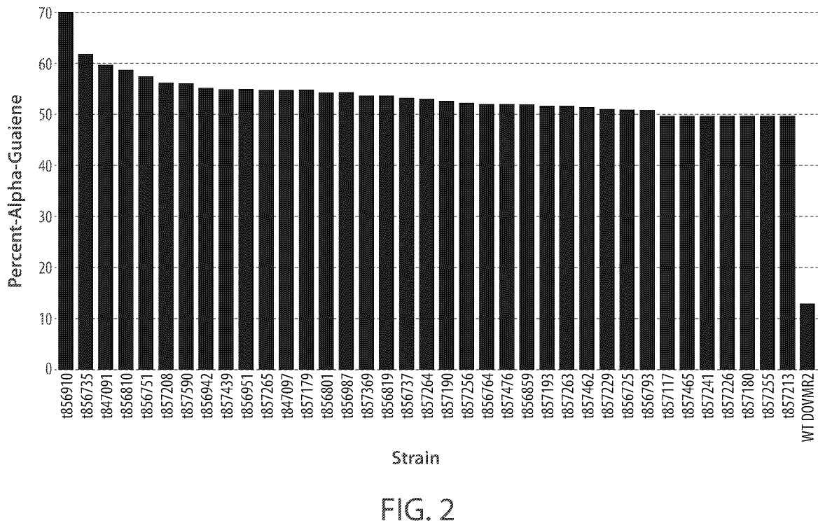

Table 4 and FIG. 2 show the top performing variant sesquiterpene synthases identified from the GC-MS screen based on percent alpha-guaiene produced. The GC-MS assay was conducted on a Thermo ISQ GC-MS, with a mass scan range of 40-350 m/z and ion source set to 250° C. The front inlet was set to 250° C., with a flow of 1.5 mL/min He and a split flow ratio of 4. The GC ramp started at 80° C. at a rate of 7.5° C./min to 200° C., followed by another ramp at a rate of 20° C. to final holding temperature 280° C. Mzmine2 and thermo xcalibur software were utilized for peak detection, spectra/product identification, and peak area reported.

Percent (%) alpha-guaiene and percent (%) delta-guaiene values represent the percent amount of the respected products of the total sesquiterpene synthase product profile detected by the assay. The percentage for each sesquiterpene detected was calculated using the extracted ion chromatogram (EIC) peak area, based on m/z fragments with the most similar peak area response (based on authentic sesquiterpene standards and NIST spectra library). For example, WT (no mutations relative to SEQ ID NO: 1) produced alpha-guaiene (105 m/z), delta-guaiene (107 m/z), aciphyllene* (105 m/z), beta-elemene (81 m/z), humulene* (93 m/z), and neointermedeol* (81 m/z) peak areas for the percent alpha-guaiene calculation (* denoting products predicted via NIST spectra library). The fold increase in alpha-guaiene production was calculated as the amount of alpha-guaiene product produced by a host cell expressing the variant sesquiterpene synthase relative to that produced by a host cell expressing a control sesquiterpene synthase comprising SEQ ID NO: 1. Thus, in Table 4: “Productivity” refers to the fold increase in alpha-guaiene production attributed to a variant enzyme relative to alpha-guaiene production attributed to the wild type sesquiterpene synthase, % alpha-guaiene refers to the amount of alpha-guaiene relative to the total sesquiterpene produced by a host cell expressing a variant enzyme, and % delta-guaiene refers to the amount of delta-guaiene relative to the total sesquiterpene produced by a variant enzyme.

TABLE 4
Alpha-guaiene and delta-guaiene production by variant sesquiterpene synthases

| | | | | | | % Non- |
|---|---|---|---|---|---|---|
| | | | | | | Alpha- |
| | | Mutations | | | | Guaiene, |
| | Sesquiterpene | relative to | | | | Non-Delta- |
| | Synthase | SEQ ID | Productivity | % Alpha- | % Delta- | Guaiene |
| Strain ID | SEQ ID NO: | NO: 1 | % | Guaiene | Guaiene | Products |
| | 1 | N/A | 1 | 14.6 | 71.7 | 13.70 |
| t856910 | 3 | N289H V292F | 22.21 | 70.44 | 21.02 | 8.54 |
| | | G293N T295V | | | | |
| | | K404W | | | | |
| | | F406L F512L | | | | |
| t856735 | 4 | N289H I291V | 27.76 | 62.17 | 31.68 | 6.15 |
| | | V292F G293Q | | | | |
| | | T295V | | | | |
| | | K404W | | | | |
| | | F406L F512L | | | | |
| t847091 | 5 | N289H F406L | 0.79 | 59.93 | 10.43 | 29.64 |
| | | F512L D191N | | | | |
| | | A205E | | | | |
| t856810 | 6 | I291V V292F | 8.40 | 58.95 | 24.06 | 16.99 |
| | | G293Q T295V | | | | |
| | | K404W | | | | |
| | | F406L I507W | | | | |
| | | F512L | | | | |
| t856751 | 7 | N289H V292F | 42.09 | 57.78 | 33.70 | 8.52 |
| | | T295V | | | | |
| | | K404W | | | | |
| | | F406L F512L | | | | |
| t857208 | 8 | N289H I291V | 28.95 | 56.44 | 33.77 | 9.79 |
| | | V292F G293N | | | | |
| | | T295V | | | | |
| | | K404W | | | | |
| | | F406L F512L | | | | |
| t857590 | 9 | V292F G293Q | 2.77 | 56.37 | 9.43 | 34.21 |
| | | K404W | | | | |
| | | F406L | | | | |
| t856942 | 10 | N289H V292F | 27.31 | 55.41 | 39.49 | 5.09 |
| | | G293I T295V | | | | |
| | | F406L I407G | | | | |
| | | F512L | | | | |
| t857439 | 11 | V292F G293Q | 2.80 | 55.25 | 17.19 | 27.56 |
| | | K404W | | | | |
| | | F406L I407G | | | | |
| | | F512L | | | | |
| t856951 | 12 | N289H I291V | 24.26 | 55.21 | 40.57 | 4.23 |
| | | V292F G293I | | | | |
| | | T295V F406L | | | | |
| | | I407G F512L | | | | |
| t857265 | 13 | V292F T295V | 33.13 | 54.95 | 38.63 | 6.41 |
| | | K404W | | | | |
| | | F406L I507W | | | | |
| | | F512L | | | | |
| t847097 | 14 | N289H | 2.25 | 54.94 | 17.85 | 27.21 |
| | | V292M G293I | | | | |
| | | F406L F512L | | | | |
| | | M480I | | | | |
| t857179 | 15 | N289H I291V | 46.72 | 54.93 | 35.11 | 9.96 |
| | | V292F G293Q | | | | |
| | | T295V | | | | |
| | | K404W | | | | |
| | | F406L | | | | |
| t856801 | 16 | V292F G293Q | 8.74 | 54.57 | 33.98 | 11.46 |
| | | T295V F406L | | | | |
| | | F512L | | | | |
| t856987 | 17 | V292F T295V | 45.60 | 54.51 | 37.86 | 7.63 |
| | | K404W | | | | |
| | | F406L F512L | | | | |
| t857369 | 18 | I291V V292F | 1.30 | 54.06 | 20.92 | 25.02 |
| | | G293I K404W | | | | |
| | | F406L I507W | | | | |
| t856819 | 19 | V292F G293I | 12.38 | 53.94 | 35.47 | 10.59 |
| | | T295V F406L | | | | |
| | | I407G F512L | | | | |
| t856737 | 20 | N289H V292F | 26.00 | 53.54 | 38.74 | 7.71 |
| | | G293I T295V | | | | |
| | | F406L I507W | | | | |
| | | F512L | | | | |
| t857264 | 21 | N289H I291V | 45.34 | 53.36 | 33.35 | 13.29 |
| | | V292F G293I | | | | |
| | | T295V | | | | |
| | | K404W | | | | |
| | | F406L F512L | | | | |
| t857190 | 22 | N289H V292F | 20.07 | 52.99 | 41.06 | 5.95 |
| | | G293Q T295V | | | | |
| | | F406L I407G | | | | |
| | | F512L | | | | |
| t857256 | 23 | N289H I291V | 24.53 | 52.56 | 34.84 | 12.60 |
| | | V292F G293N | | | | |
| | | T295V | | | | |
| | | K404W | | | | |
| | | F406L | | | | |
| t856764 | 24 | N289H V292F | 14.12 | 52.36 | 45.25 | 2.39 |
| | | G293Q F406L | | | | |
| | | I507W F512L | | | | |
| t857476 | 25 | I291V V292F | 4.78 | 52.31 | 17.33 | 30.36 |
| | | G293N T295V | | | | |
| | | K404W | | | | |
| | | F406L I507W | | | | |
| | | F512L | | | | |
| t856859 | 26 | N289H V292F | 7.66 | 52.26 | 34.23 | 13.51 |
| | | G293N | | | | |
| | | K404W | | | | |
| | | F406L I507W | | | | |
| | | F512L | | | | |
| t857193 | 27 | N289H V292F | 19.00 | 52.02 | 39.62 | 8.35 |
| | | G293I T295V | | | | |
| | | F406L I407G | | | | |
| | | I507W F512L | | | | |
| t857263 | 28 | N289H I291V | 17.82 | 51.96 | 41.36 | 6.68 |
| | | V292F G293N | | | | |
| | | T295V F406L | | | | |
| | | F512L | | | | |
| t857462 | 29 | I291V V292F | 3.84 | 51.81 | 24.83 | 23.35 |
| | | G293Q | | | | |
| | | K404W | | | | |
| | | F406L I407G | | | | |
| | | F512L | | | | |
| t857229 | 30 | I291V V292F | 8.92 | 51.42 | 30.60 | 17.97 |
| | | G293Q T295V | | | | |
| | | K404W | | | | |
| | | F406L I407G | | | | |
| | | F512L | | | | |
| t856725 | 31 | N289H V292F | 33.94 | 51.19 | 43.89 | 4.92 |
| | | T295V F406L | | | | |
| | | F512L | | | | |
| t856793 | 32 | N289H V292F | 12.70 | 51.13 | 36.20 | 12.67 |
| | | K404W | | | | |
| | | F406L F512L | | | | |
| 1857117 | 33 | V292F G293N | 9.84 | 50.92 | 41.23 | 7.84 |
| | | T295V F406L | | | | |
| | | F512L | | | | |
| t857465 | 34 | N289H I291V | 2.81 | 50.57 | 20.24 | 29.19 |
| | | V292F G293N | | | | |
| | | T295V F406L | | | | |
| | | I407G I507W | | | | |
| | | F512L | | | | |
| t857241 | 35 | N289H I291V | 48.80 | 50.54 | 44.54 | 4.92 |
| | | V292F G293I | | | | |
| | | T295V F406L | | | | |
| | | F512L | | | | |
| t857226 | 36 | N289H I291V | 77.54 | 50.25 | 43.83 | 5.93 |
| | | V292F G293Q | | | | |
| | | T295V F406L | | | | |
| | | I507W | | | | |
| t857180 | 37 | N289H V292F | 22.11 | 50.21 | 41.63 | 8.16 |
| | | G293I F406L | | | | |
| | | I407G I507W | | | | |
| | | F512L | | | | |
| t857255 | 38 | N289H V292F | 9.64 | 50.13 | 31.34 | 18.53 |
| | | G293Q T295V | | | | |
| | | K404W | | | | |
| | | F406L I507W | | | | |
| t857213 | 39 | I291V V292F | 9.66 | 50.04 | 39.78 | 10.18 |
| | | G293Q T295V | | | | |
| | | F406L I507W | | | | |
| | | F512L | | | | |

Surprisingly, as shown in Table 4 and FIG. 2, variant sesquiterpene synthases were identified that produce more than 50% alpha-guaiene, in contrast to the Delta-guaiene synthase 2 protein from A. crassna (UniProt Accession No. D0VMR7; SEQ ID NO: 1), which was found to produce 14.6% alpha-guaiene.

Following identification of variant sesquiterpene synthases that produced increased amounts of alpha-guaiene, a linear model was used to predict which specific amino acid substitution mutations contributed the most to the apparent increase in alpha-guaiene production. After the linear model was fit, coefficients of the model associated with specific amino acid substitution mutations that were positive and large in magnitude, denoting an increased statistical correlation with alpha-guaiene production, were identified. Table 5 shows the amino acid substitutions statistically correlated with alpha-guaiene production based on this analysis.

TABLE 5
Amino acid substitutions positively associated with alpha-guaiene production

| Residue in SEQ ID NO: 1 | Amino Acid Substitution |
|---|---|
| V292 | A, Q, C, Y, H, E, F, T, W |
| N289 | L, T, S, D, M |
| Y403 | M, Q, N, V, C, H, E, A, T, S |
| T295 | E, D, N, W, G, I |
| D444 | A, N |
| I291 | F, L, T, C |
| I294 | L, C, Y, V, A, S |
| S296 | L, A, P, Y, N, R, F |
| V515 | H, A, E, Q |
| G293 | L, V, T, Y, C, F, W, M |
| L297 | E, Y, I, K, M, H |
| G525 | H, C, L, N |
| F406 | L, T, W, N |

Without wishing to be bound by any theory, the shape of the active site pocket of the sesquiterpene synthase may influence the ratio of different products produced by the sesquiterpene synthase. In the reaction catalyzed by the Delta-guaiene synthase 2 protein from A. crassna (UniProt Accession No. D0VMR7; SEQ ID NO: 1), the final step involves a proton abstraction from either the pro-delta carbon, leading to delta-guaiene as the product, or the pro-alpha carbon, leading to alpha-guaiene as the product. Some variant sesquiterpene synthases may produce increased amounts of alpha-guaiene at least in part because they alter the substrate binding mode of the sesquiterpene synthase to allow easier access to the pro-alpha carbon from the catalytic residue Tyr-520.

### Example 2: Identification of Additional Variant Sesquiterpene Synthases that Produce Increased Alpha-Guaiene

To identify additional variant sesquiterpene synthases capable of producing increased amounts of alpha-guaiene, additional variant versions of Delta-guaiene synthase 2 protein from A. crassna (UniProt Accession No. D0VMR7; SEQ ID NO: 1) were produced and screened.

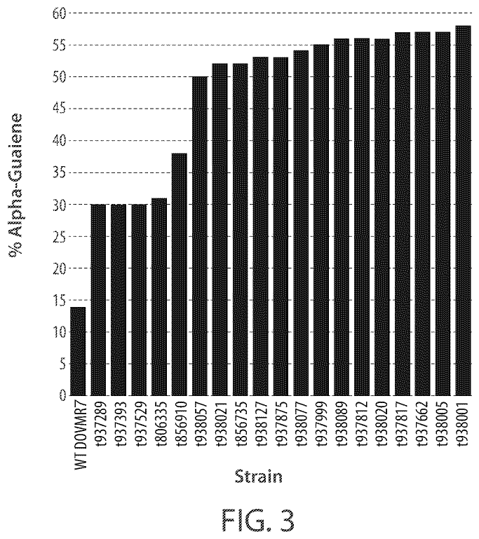

Using methods described in Example 1, variant sesquiterpene synthases were screened for alpha-guaiene production in a primary screen. This primary screen employed a GC-MS analysis to identify top performing variant sesquiterpene synthases using methods described in O'Maille et al. (2008) Nat. Chem. Biol. 4, 617-623. Approximately 1012 variant sesquiterpene synthases were screened. Nineteen high-performing variant sesquiterpene synthases were identified in the primary screen, including three variant sesquiterpene synthases from Example 1. The nineteen variant sesquiterpene synthases were further characterized in a secondary screen using a GC-MS assay based on methods described in O'Maille et al. (2008) Nat. Chem. Biol. 4, 617-623, but with longer ramp speeds than were used in the primary screen. (Table 6 and FIG. 3). For the secondary screening GC-MS method, inlet temperature was set to 250° C. with a flow rate of 1.5 mL/min respectively using a TG5-MS column (15m×0.250 mm×0.25 μm) on a Thermo GC-MS-ISQ. The oven was set to 80.0° C. with an initial hold of 0.1 minutes, a 7.5° C./min ramp to 200.0° C., and a 20.0° C./min ramp to 280° C. with a hold of 1.00 minutes. MS transfer line temperature was set to 280° C. Ion source temperature was set to 250° C. Mass range was set to 40-350 amu.

A tertiary screen was performed on a subset of strains using GC-FID, which provides a quantitative read-out with higher accuracy than GC-MS, based on methods described in Greenhagen et al. (2006) Proc. Natl. Acad. Sci. 103 (26) 9826-9831. The results confirmed identification of variant sesquiterpene synthases with increased alpha-guaiene production compared to that produced by a positive control strain expressing the Delta-guaiene synthase 2 protein from A. crassna (UniProt Accession No. D0VMR7; SEQ ID NO: 1) (Table 6).

Table 6 shows results from the primary, secondary, and tertiary screens. “% Alpha-Guaiene (Primary Screen)” represents the percent amount of alpha-guaiene product of the total sesquiterpene synthase product profile detected by the primary screen assay. “% Alpha-Guaiene (Secondary Screen)” represents the percent amount of alpha-guaiene product of the total sesquiterpene synthase product profile detected by the secondary screen assay. “% Alpha-Guaiene (Tertiary Screen)” represents the percent amount of alpha-guaiene product of the total sesquiterpene synthase product profile detected by the tertiary screen assay. “% Other Sesquiterpenes relative to Tertiary Screen” represents the percent amount of non-alpha-guaiene product of the total sesquiterpene synthase product profile detected by the tertiary screen assay.

TABLE 6
Alpha-guaiene production by variant sesquiterpene synthases

| | | | | | | % Other |
|---|---|---|---|---|---|---|
| | | | % Alpha- | % Alpha- | % Alpha- | Sesquiterpenes |
| | Sesquiterpene | Mutations | Guaiene | Guaiene | Guaiene | relative to |
| Strain | Synthase | relative to | (Primary | (Secondary | (Tertiary | Tertiary |
| ID | SEQ ID NO: | SEQ ID NO: 1 | Screen) | Screen) | Screen) | Screen |
| t516637 | 1 | N/A | 12.00 | 14.00 | 14.00 | 86.00 |
| t938005 | 77 | N289H V292F | 64.00 | 57.00 | 65.00 | 35.00 |
| | | G293N T295V | | | | |
| | | K404W F406L | | | | |
| | | Y442L F512L | | | | |
| t938001 | 78 | N289H I291V | 64.00 | 58.00 | 67.00 | 33.00 |
| | | V292F G293Q | | | | |
| | | T295V K404W | | | | |
| | | F406L Y442L | | | | |
| | | F512L | | | | |
| t938020 | 79 | T72I N289H I291V | 66.00 | 56.00 | 60.00 | 40.00 |
| | | V292F G293Q | | | | |
| | | T295V K404W | | | | |
| | | F406L T494E | | | | |
| | | F512L | | | | |
| t937812 | 80 | P124S N289H | 62.00 | 56.00 | 61.00 | 39.00 |
| | | I291V V292F | | | | |
| | | G293Q T295V | | | | |
| | | K404W F406L | | | | |
| | | F512L | | | | |
| t937875 | 81 | P124S N289H | 62.00 | 53.00 | N/A | N/A |
| | | I291V V292F | | | | |
| | | G293Q T295V | | | | |
| | | K404W F406L | | | | |
| t937817 | 82 | T153S N289H | 65.00 | 57.00 | 58.00 | 42.00 |
| | | I291V V292F | | | | |
| | | G293Q T295V | | | | |
| | | D346E K404W | | | | |
| | | F406L F512L | | | | |
| t937999 | 83 | R290K V292F | 60.00 | 55.00 | N/A | N/A |
| | | T295V K404W | | | | |
| | | F406L F512L | | | | |
| t937662 | 84 | S122N N289H | 59.00 | 57.00 | N/A | N/A |
| | | I291V V292F | | | | |
| | | G293Q T295V | | | | |
| | | K404W F406L | | | | |
| | | F512L | | | | |
| t938057 | 7 | N289H V292F | 60.00 | 50.00 | N/A | N/A |
| | | T295V K404W | | | | |
| | | F406L F512L | | | | |
| t938089 | 85 | P124S V292F | 62.00 | 56.00 | N/A | N/A |
| | | T295V K404W | | | | |
| | | F406L I507W | | | | |
| | | F512L | | | | |
| t938077 | 86 | T72I N289H V292F | 59.00 | 54.00 | N/A | N/A |
| | | G293I T295V | | | | |
| | | F406L I407G | | | | |
| | | Y442L I507W | | | | |
| | | F512L | | | | |
| t938127 | 87 | N289H V292F | 59.00 | 53.00 | N/A | N/A |
| | | T295V K404W | | | | |
| | | F406L Y442L | | | | |
| | | F512L | | | | |
| t938021 | 88 | T72I N289H V292F | 60.00 | 52.00 | N/A | N/A |
| | | T295V K404W | | | | |
| | | F406L T494E | | | | |
| | | F512L | | | | |
| t856735 | 4 | N289H I291V | 58.00 | 52.00 | N/A | N/A |
| | | V292F G293Q | | | | |
| | | T295V K404W | | | | |
| | | F406L F512L | | | | |
| t856910 | 3 | N289H V292F | 52.00 | 38.00 | N/A | N/A |
| | | G293N T295V | | | | |
| | | K404W F406L | | | | |
| | | F512L | | | | |
| t806335 | 89 | N289H F406L | 35.00 | 31.00 | N/A | N/A |
| | | F512L T295V | | | | |
| t937289 | 90 | N289H F406L | 33.00 | 30.00 | N/A | N/A |
| | | F512L T295V | | | | |
| | | G274S | | | | |
| t937529 | 91 | N289H F406L | 33.00 | 30.00 | N/A | N/A |
| | | F512L T295V | | | | |
| | | V201I | | | | |
| t937393 | 92 | N289H F406L | 32.00 | 30.00 | N/A | N/A |
| | | F512L T295V | | | | |
| | | I398C | | | | |

As shown in Table 6, variant sesquiterpene synthases were surprisingly identified that produce more than 60% alpha-guaiene, in contrast to the Delta-guaiene synthase 2 protein from A. crassna (UniProt Accession No. D0VMR7; SEQ ID NO: 1), which was found to produce ˜13% alpha-guaiene.

### Example 3. Identification of Additional Variant Sesquiterpene Synthases that Produce Increased Alpha-Guaiene

To identify additional variant sesquiterpene synthases capable of producing increased amounts of alpha-guaiene, additional variants of Delta-guaiene synthase 2 protein from A. crassna (UniProt Accession No. D0VMR7; SEQ ID NO: 1) were generated.

Using methods described in Example 1, variant sesquiterpene synthases were screened for alpha-guaiene production in a primary screen as follows. Yeast expression plasmid vectors containing variant sesquiterpene synthase DNA coding sequences flanked by a GAL1 promoter and terminator were transformed into screening strains for in vivo evaluation of enzymatic performance. The screening strains were engineered to express higher concentrations of farnesyl diphosphate (FPP), which serves as a substrate for the enzymes being screened. Within the screening strains, the sesquiterpene synthases turn over the FPP when galactose is available. The GAL1 promoter is responsive to galactose and induces variant enzyme expression in the presence of galactose. After sufficient exposure to galactose, the strains were harvested and exposed to an organic solvent, which was used to isolate the reaction products. Once isolated, the reaction products were analyzed by GC to determine the relative abundance, proportions, and identity of products in the reaction product mix.

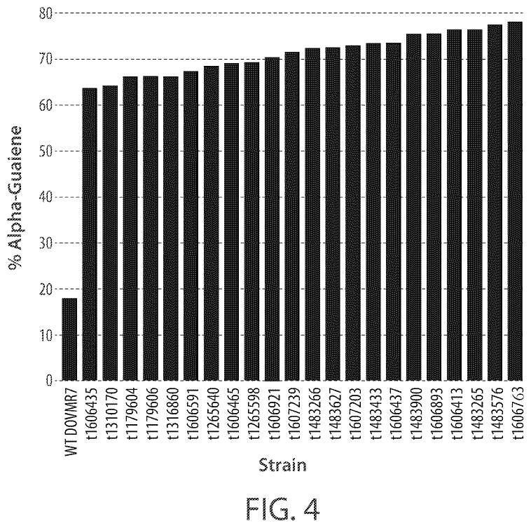

This primary screen employed a GC-MS analysis to identify top performing variant sesquiterpene synthases using methods described in O'Maille et al. (2008) Nat. Chem. Biol. 4, 617-623. Approximately 5000 variant sesquiterpene synthases were screened through multiple screens. Twenty three high-performing variant sesquiterpene synthases were identified in the primary screen. The twenty three variant sesquiterpene synthases were further characterized in a secondary screen using a GC-MS assay based on methods described in O'Maille et al. (2008) Nat. Chem. Biol. 4,617-623, but with longer ramp speeds than were used in the primary screen. (Table 7 and FIG. 4). For the secondary screening GC-MS method, inlet temperature was set to 250° C. with a flow rate of 1.5 mL/min respectively using a TG5-MS column (15m×0.250 mm×0.25 μm) on a Thermo GC-MS-ISQ. The oven was set to 80.0° C. with an initial hold of 0.1 minutes, a 7.5° C./min ramp to 200.0° C., and a 20.0° C./min ramp to 280° C. with a hold of 1.00 minutes. MS transfer line temperature was set to 280° C. Ion source temperature was set to 250° C. Mass range was set to 40-350 amu.

The results confirmed identification of variant sesquiterpene synthases with increased alpha-guaiene production compared to that produced by a positive control strain expressing the Delta-guaiene synthase 2 protein from A. crassna (UniProt Accession No. D0VMR7; SEQ ID NO: 1) (Table 7).

Table 7 shows results from the primary and secondary screens. “% Alpha-Guaiene (Primary Screen)” represents the percent amount of alpha-guaiene product of the total sesquiterpene synthase product profile detected by the primary screen assay. “% Alpha-Guaiene (Secondary Screen)” represents the percent amount of alpha-guaiene product of the total sesquiterpene synthase product profile detected by the secondary screen assay. “% Alpha-Guaiene (Tertiary Screen)” represents the percent amount of alpha-guaiene product of the total sesquiterpene synthase product profile detected by the tertiary screen assay. “% Other Sesquiterpenes relative to Tertiary Screen” represents the percent amount of non-alpha-guaiene product and non-aciphyllene product of the total sesquiterpene synthase product profile detected by the tertiary screen assay.

TABLE 7
Alpha-guaiene production by variant sesquiterpene synthases

| | | | | | % | | | | % Other |
|---|---|---|---|---|---|---|---|---|---|
| | | Mutations | % Alpha- | % | Alpha- | % | % Alpha- | % | Sesquiterpenes |
| | Sesquiterpene | relative to | Guaiene | Aciphyllene | Guaiene | Aciphyllene | Guaiene | Aciphyllene | relative to |
| | Synthase | SEQ ID | (Primary | (Primary | (Secondary | (Secondary | (Tertiary | (Tertiary | Tertiary |
| Strain ID | SEQ ID NO: | NO: 1 | Screen) | Screen) | Screen) | Screen) | Screen) | Screen) | Screen |
| t516637 | 1 | N/A | 13.00 | N/A | 17.70 | N/A | N/A | N/A | N/A |
| t1179604 | 110 | T72I | 85.00 | <3.00 | 65.00 | <3.00 | N/A | N/A | N/A |
| | | N289H | | | | | | | |
| | | R290K | | | | | | | |
| | | I291V | | | | | | | |
| | | V292F | | | | | | | |
| | | G293Q | | | | | | | |
| | | T295V | | | | | | | |
| | | K404W | | | | | | | |
| | | F406L | | | | | | | |
| | | Y442L | | | | | | | |
| | | T494E | | | | | | | |
| | | F512L | | | | | | | |
| t1179606 | 111 | L44V T72I | 81.00 | <3.00 | 65.00 | <3.00 | N/A | N/A | N/A |
| | | N289H | | | | | | | |
| | | I291V | | | | | | | |
| | | V292F | | | | | | | |
| | | G293Q | | | | | | | |
| | | T295V | | | | | | | |
| | | K404W | | | | | | | |
| | | F406L | | | | | | | |
| | | Y442L | | | | | | | |
| | | F512L | | | | | | | |
| t1310170 | 112 | L44V T72I | 60.00 | <3.00 | 63.00 | <3.00 | N/A | N/A | N/A |
| | | T86E | | | | | | | |
| | | G118Q | | | | | | | |
| | | T134D | | | | | | | |
| | | W147L | | | | | | | |
| | | Q212F | | | | | | | |
| | | Q217E | | | | | | | |
| | | S224L | | | | | | | |
| | | F252L | | | | | | | |
| | | P255A | | | | | | | |
| | | N289H | | | | | | | |
| | | R290K | | | | | | | |
| | | I291V | | | | | | | |
| | | V292F | | | | | | | |
| | | G293Q | | | | | | | |
| | | T295V | | | | | | | |
| | | D346E | | | | | | | |
| | | Y390F | | | | | | | |
| | | K404W | | | | | | | |
| | | F406L | | | | | | | |
| | | L419S | | | | | | | |
| | | Y442L | | | | | | | |
| | | T458V | | | | | | | |
| | | R467K | | | | | | | |
| | | F512L | | | | | | | |
| | | R542V | | | | | | | |
| t1316860 | 113 | T23D | 65.00 | <3.00 | 65.00 | <3.00 | N/A | N/A | N/A |
| | | L44V T72I | | | | | | | |
| | | N289H | | | | | | | |
| | | I291V | | | | | | | |
| | | V292F | | | | | | | |
| | | G293Q | | | | | | | |
| | | T295V | | | | | | | |
| | | K404W | | | | | | | |
| | | F406L | | | | | | | |
| | | Y442L | | | | | | | |
| | | F512L | | | | | | | |
| t1265598 | 114 | V201S | 68.00 | <3.00 | 68.00 | <3.00 | N/A | N/A | N/A |
| | | N289H | | | | | | | |
| | | I291V | | | | | | | |
| | | V292F | | | | | | | |
| | | G293N | | | | | | | |
| | | T295V | | | | | | | |
| | | Y381W | | | | | | | |
| | | K404W | | | | | | | |
| | | F406L | | | | | | | |
| | | T494E | | | | | | | |
| | | F512L | | | | | | | |
| t1265640 | 115 | V201S | 65.00 | <3.00 | 67.10 | <3.00 | N/A | N/A | N/A |
| | | V292C | | | | | | | |
| | | T295I | | | | | | | |
| | | D444N | | | | | | | |
| t1483433 | 116 | V201S | 64.00 | 18.20 | 72.00 | 19.50 | 68.00 | 26.00 | 6.00 |
| | | V292C | | | | | | | |
| | | T295I | | | | | | | |
| | | D444N | | | | | | | |
| | | L516V | | | | | | | |
| t1483627 | 117 | L44V T72I | 62.00 | 19.70 | 71.00 | 19.20 | 68.50 | 24.50 | 7.00 |
| | | V201S | | | | | | | |
| | | V292C | | | | | | | |
| | | T295I | | | | | | | |
| | | D444N | | | | | | | |
| | | L516V | | | | | | | |
| t1483266 | 118 | L44V T72I | 62.00 | 21.60 | 71.00 | 20.50 | 68.50 | 26.00 | 5.50 |
| | | V201S | | | | | | | |
| | | V292C | | | | | | | |
| | | T295I | | | | | | | |
| | | D444N | | | | | | | |
| | | L516A | | | | | | | |
| t1483265 | 119 | L44V T72I | 68.00 | 21.70 | 75.00 | 13.30 | 70.00 | 25.00 | 5.00 |
| | | V201S | | | | | | | |
| | | V292C | | | | | | | |
| | | T295I | | | | | | | |
| | | I443W | | | | | | | |
| | | D444N | | | | | | | |
| t1483900 | 120 | L44V T72I | 67.00 | 17.20 | 74.00 | 20.30 | 71.50 | 25.00 | 3.50 |
| | | V201S | | | | | | | |
| | | V292C | | | | | | | |
| | | T295I | | | | | | | |
| | | D444N | | | | | | | |
| | | M519L | | | | | | | |
| t1483576 | 121 | W147V | 66.00 | 25.00 | 76.00 | 23.60 | 65.00 | 29.50 | 5.50 |
| | | C188Q | | | | | | | |
| | | V201S | | | | | | | |
| | | S224L | | | | | | | |
| | | V292C | | | | | | | |
| | | T295I | | | | | | | |
| | | D444N | | | | | | | |
| t1606763 | 122 | L44V T72I | 74.5.00 | 12.80 | 76.70 | 13.60 | N/A | N/A | N/A |
| | | V111L | | | | | | | |
| | | V201S | | | | | | | |
| | | S224L | | | | | | | |
| | | V292C | | | | | | | |
| | | T295I | | | | | | | |
| | | I443T | | | | | | | |
| | | D444N | | | | | | | |
| | | T448G | | | | | | | |
| | | M519L | | | | | | | |
| t1606435 | 123 | L44V T72I | 72.00 | 15.80 | 62.50 | 18.50 | N/A | N/A | N/A |
| | | V111L | | | | | | | |
| | | V201S | | | | | | | |
| | | V292C | | | | | | | |
| | | T295I | | | | | | | |
| | | V433I | | | | | | | |
| | | I443T | | | | | | | |
| | | D444N | | | | | | | |
| | | T448G | | | | | | | |
| | | D499N | | | | | | | |
| | | L516V | | | | | | | |
| t1606893 | 124 | L44V T72I | 75.60 | 13.35 | 74.00 | 13.80 | N/A | N/A | N/A |
| | | V201S | | | | | | | |
| | | V292C | | | | | | | |
| | | T295I | | | | | | | |
| | | D444N | | | | | | | |
| | | L516V | | | | | | | |
| | | M519L | | | | | | | |
| t1606413 | 125 | L44V T72I | 89.60 | 16.30 | 74.95 | 17.20 | N/A | N/A | N/A |
| | | V201S | | | | | | | |
| | | V292C | | | | | | | |
| | | T295I | | | | | | | |
| | | D444N | | | | | | | |
| | | V515M | | | | | | | |
| | | L516V | | | | | | | |
| t1606591 | 126 | V111L | 77.60 | 18.19 | 66.00 | 18.10 | N/A | N/A | N/A |
| | | V201S | | | | | | | |
| | | S224L | | | | | | | |
| | | V292C | | | | | | | |
| | | T295I | | | | | | | |
| | | I443T | | | | | | | |
| | | D444N | | | | | | | |
| | | L516V | | | | | | | |
| t1607203 | 127 | V201S | 71.50 | 17.54 | 71.60 | 17.50 | N/A | N/A | N/A |
| | | V292C | | | | | | | |
| | | T295I | | | | | | | |
| | | D444N | | | | | | | |
| | | L516V | | | | | | | |
| t1607239 | 128 | V201S | 70.50 | 18.40 | 70.10 | 16.60 | N/A | N/A | N/A |
| | | V292C | | | | | | | |
| | | T295I | | | | | | | |
| | | D444N | | | | | | | |
| | | T458L | | | | | | | |
| | | L516A | | | | | | | |
| t1606465 | 129 | V201S | 79.00 | 17.90 | 67.80 | 18.00 | N/A | N/A | N/A |
| | | V292C | | | | | | | |
| | | T295I | | | | | | | |
| | | I443T | | | | | | | |
| | | D444N | | | | | | | |
| | | L516V | | | | | | | |
| t1606437 | 130 | V201S | 78.80 | <3.00 | 72.20 | 18.40 | N/A | N/A | N/A |
| | | V292C | | | | | | | |
| | | T295I | | | | | | | |
| | | V433I | | | | | | | |
| | | D444N | | | | | | | |
| | | L516V | | | | | | | |
| t1606921 | 131 | V201S | 71.00 | 18.40 | 69.00 | 17.20 | N/A | N/A | N/A |
| | | V292C | | | | | | | |
| | | T295I | | | | | | | |
| | | V433L | | | | | | | |
| | | D444N | | | | | | | |
| | | L516V | | | | | | | |

As shown in Table 7, variant sesquiterpene synthases were surprisingly identified that produced more than 70% alpha-guaiene, in contrast to the Delta-guaiene synthase 2 protein from A. crassna (UniProt Accession No. D0VMR7; SEQ ID NO: 1), which was found to produce ˜15% alpha-guaiene. In addition, variant sesquiterpene synthases were surprisingly identified that produced aciphyllene as the second most abundant sesquiterpene product.

Table 8 includes protein and nucleic acid sequences for sesquiterpene synthase variants associated with the disclosure.

In some embodiments, a sesquiterpene synthase comprises a sequence that is at least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to any one of SEQ ID NOs: 3-39, 77-92 or 110-131, a protein sequence within Table 8, or a conservatively substituted variant thereof. In certain embodiments, a sesquiterpene synthase comprises the sequence of any one of SEQ ID NOs: 3-39, 77-92 or 110-131 or a conservatively substituted version thereof. In certain embodiments a sesquiterpene synthase consists of or consists essentially of the sequence of any one of SEQ ID NOs: 3-39, 77-92, 110-131 or a conservatively substituted variant thereof.

In some embodiments, a polynucleotide encoding a sesquiterpene synthase comprises a sequence that is at least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to any one of SEQ ID NOs: 40-76, 93-109, 133-154 or a nucleic acid sequence within Table 8. In certain embodiments, a polynucleotide encoding a sesquiterpene synthase comprises any one of SEQ ID NOs: 40-76, 93-109, 133-154. In certain embodiments a polynucleotide encoding a sesquiterpene synthase consists of or consists essentially of any one of SEQ ID NOs: 40-76, 93-109, 133-154.

TABLE 8
Sequences of sesquiterpenes described in Examples 1-3

| | Amino Acid Sequence | Nucleotide Sequence |
|---|---|---|
| Strain ID | SEQ ID NO: | SEQ ID NO: |
| t516637 | 1 | 2 |
| t856910 | 3 | 40 |
| t856735 | 4 | 41 |
| t847091 | 5 | 42 |
| t856810 | 6 | 43 |
| t856751 | 7 | 44 |
| t857208 | 8 | 45 |
| t857590 | 9 | 46 |
| t856942 | 10 | 47 |
| t857439 | 11 | 48 |
| t856951 | 12 | 49 |
| t857265 | 13 | 50 |
| t847097 | 14 | 51 |
| t857179 | 15 | 52 |
| t856801 | 16 | 53 |
| t856987 | 17 | 54 |
| t857369 | 18 | 55 |
| t856819 | 19 | 56 |
| t856737 | 20 | 57 |
| t857264 | 21 | 58 |
| t857190 | 22 | 59 |
| t857256 | 23 | 60 |
| t856764 | 24 | 61 |
| t857476 | 25 | 62 |
| t856859 | 26 | 63 |
| t857193 | 27 | 64 |
| t857263 | 28 | 65 |
| t857462 | 29 | 66 |
| t857229 | 30 | 67 |
| t856725 | 31 | 68 |
| t856793 | 32 | 69 |
| t857117 | 33 | 70 |
| t857465 | 34 | 71 |
| t857241 | 35 | 72 |
| t857226 | 36 | 73 |
| t857180 | 37 | 74 |
| t857255 | 38 | 75 |
| t857213 | 39 | 76 |
| t938005 | 77 | 93 |
| t938001 | 78 | 94 |
| t938020 | 79 | 95 |
| t937812 | 80 | 96 |
| t937875 | 81 | 97 |
| t937817 | 82 | 98 |
| t937999 | 83 | 99 |
| t937662 | 84 | 100 |
| t938089 | 85 | 101 |
| t938077 | 86 | 102 |
| t938127 | 87 | 103 |
| t938021 | 88 | 104 |
| t806335 | 89 | 105 |
| t937289 | 90 | 106 |
| t937529 | 91 | 107 |
| t937393 | 92 | 108 |
| t938057 | 7 | 109 |
| t1179604 | 110 | 133 |
| t1179606 | 111 | 134 |
| t1310170 | 112 | 135 |
| t1316860 | 113 | 136 |
| t1265598 | 114 | 137 |
| t1265640 | 115 | 138 |
| t1483433 | 116 | 139 |
| t1483627 | 117 | 140 |
| t1483266 | 118 | 141 |
| t1483265 | 119 | 142 |
| t1483900 | 120 | 143 |
| t1483576 | 121 | 144 |
| t1606763 | 122 | 145 |
| t1606435 | 123 | 146 |
| t1606893 | 124 | 147 |
| t1606413 | 125 | 148 |
| t1606591 | 126 | 149 |
| t1607203 | 127 | 150 |
| t1607239 | 128 | 151 |
| t1606465 | 129 | 152 |
| t1606437 | 130 | 153 |
| t1606921 | 131 | 154 |

### EQUIVALENTS

Those skilled in the art will recognize, or be able to ascertain using no more than routine experimentation, many equivalents to the specific embodiments of the invention described in this disclosure. Such equivalents are intended to be encompassed by the following claims. All references, including patent documents, disclosed in this application are incorporated by reference in their entirety, particularly for the disclosure referenced in this application.

It should be appreciated that sequences disclosed in this application may or may not contain secretion signals. The sequences disclosed in this application encompass versions with or without secretion signals. It should also be understood that amino acid sequences disclosed in this application may be depicted with or without a start codon (M). The sequences disclosed in this application encompass versions with or without start codons. Accordingly, in some instances amino acid numbering may correspond to amino acid sequences containing secretion signal and/or a start codon, while in other instances, amino acid numbering may correspond to amino acid sequences that do not contain a secretion signal and/or a start codon. It should also be understood that sequences disclosed in this application may be depicted with or without a stop codon. The sequences disclosed in this application encompass versions with or without stop codons.

1. A host cell that comprises a heterologous polynucleotide encoding a sesquiterpene synthase, wherein the sequence of the sesquiterpene synthase comprises one or more amino acid substitutions relative to SEQ ID NO: 1, and wherein at least one of the amino acid substitutions is at a position corresponding to position 122, 274, 275, 293, 295, 301, 368, 398, 404, 407, 507, 515, 525, and/or 533 in SEQ ID NO: 1.

2. The host cell of claim 1, wherein the sequence of the sesquiterpene synthase further comprises at least one amino acid substitution at a position corresponding to position 72, 124, 153, 191, 201, 205, 289, 290, 291, 292, 346, 406, 442, 480, 494, 509, 512, and/or 526 in SEQ ID NO: 1.

3. A host cell that comprises a heterologous polynucleotide encoding a sesquiterpene synthase, wherein the sequence of the sesquiterpene synthase comprises two or more amino acid substitutions relative to SEQ ID NO: 1, wherein:
(i) at least one of the amino acid substitutions is at a position corresponding to position 122, 274, 275, 293, 295, 301, 368, 398, 404, 407, 507, 515, 525, and/or 533 in SEQ ID NO: 1; and
(ii) at least one of the amino acid substitutions is at a position corresponding to position 72, 124, 153, 191, 201, 205, 289, 290, 291, 292, 346, 406, 442, 480, 494, 509, 512, and/or 526 in SEQ ID NO: 1.

4. The host cell of any one of claims 1-3, wherein the host cell is capable of producing sesquiterpene products, and wherein at least 50% of the total sesquiterpene products produced by the host cell is alpha-guaiene.

5. The host cell of claim 4, wherein at least 15% of the total sesquiterpene products produced by the host cell is aciphyllene.

6. A host cell that comprises a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, wherein the two or more amino acid substitutions are at positions corresponding to position 72, 122, 124, 153, 191, 201, 205, 274, 275, 289, 290, 291, 292, 293, 295, 301, 346, 368, 398, 404, 406, 407, 442, 480, 494, 507, 509, 512, 526 and/or 533 in SEQ ID NO: 1, wherein the host cell is capable of producing sesquiterpene products, and wherein at least 50% of the total sesquiterpene products produced by the host cell is alpha-guaiene.

7. The host cell of any one of claims 1-6, wherein the sesquiterpene synthase comprises 4, 5, 6, 7, 8, 9, or more than 9 amino acid substitutions relative to SEQ ID NO: 1.

8. The host cell of any one of claims 1-7, wherein the sesquiterpene synthase comprises:
a) an isoleucine (I) residue at a position corresponding to position 72 in SEQ ID NO: 1;
b) an asparagine (N) residue at a position corresponding to position 122 in SEQ ID NO: 1;
c) a serine(S) residue at a position corresponding to position 124 in SEQ ID NO: 1;
d) an S residue at a position corresponding to position 153 in SEQ ID NO: 1;
e) an N residue at a position corresponding to position 191 in SEQ ID NO: 1;
f) an I residue at a position corresponding to position 201 of SEQ ID NO: 1;
g) a glutamate (E) residue at a position corresponding to position 205 in SEQ ID NO: 1;
h) an S residue at a position corresponding to position 274 in SEQ ID NO: 1;
i) a glycine (G) residue at a position corresponding to position 275 in SEQ ID NO: 1;
j) a histidine (H) residue at a position corresponding to position 289 in SEQ ID NO: 1;
k) a lysine (K) residue at a position corresponding to position 290 in SEQ ID NO: 1;
l) a valine (V) residue at a position corresponding to position 291 in SEQ ID NO: 1;
m) a phenylalanine (F) or methionine (M) residue at a position corresponding to position 292 in SEQ ID NO: 1;
n) a glutamine (Q), I, or N residue at a position corresponding to position 293 in SEQ ID NO: 1;
o) a V residue at a position corresponding to position 295 in SEQ ID NO: 1;
p) an S residue at a position corresponding to position 301 in SEQ ID NO: 1;
q) an E residue at a position corresponding to position 346 in SEQ ID NO: 1;
r) a cysteine (C) residue at a position corresponding to position 368 in SEQ ID NO: 1;
s) a C residue at a position corresponding to position 398 in SEQ ID NO: 1;
t) a tryptophan (W) residue at a position corresponding to position 404 in SEQ ID NO: 1;
u) a leucine (L) residue at a position corresponding to position 406 in SEQ ID NO: 1;
v) a G residue at a position corresponding to position 407 in SEQ ID NO: 1;
w) an L residue at a position corresponding to position 442 in SEQ ID NO: 1;
x) an I residue at a position corresponding to position 480 in SEQ ID NO: 1;
y) an E residue at a position corresponding to position 494 in SEQ ID NO: 1;
z) a tryptophan (W) residue at a position corresponding to position 507 in SEQ ID NO: 1;
aa) an alanine (A) residue at a position corresponding to position 509 in SEQ ID NO: 1;
bb) an L residue at a position corresponding to position 512 in SEQ ID NO: 1;
cc) an F residue at a position corresponding to position 526 in SEQ ID NO: 1;
dd) an N residue at a position corresponding to position 533 in SEQ ID NO: 1; or
ee) any combination thereof.

9. The host cell of any one of claims 1-7, wherein the sesquiterpene synthase comprises:
a) an I residue at a position corresponding to position 72 in SEQ ID NO: 1;
b) an N residue at a position corresponding to position 122 in SEQ ID NO: 1;
c) an S residue at a position corresponding to position 124 in SEQ ID NO: 1;
d) an S residue at a position corresponding to position 153 in SEQ ID NO: 1;
e) an N residue at a position corresponding to position 191 in SEQ ID NO: 1;
f) an I residue at a position corresponding to position 201 in SEQ ID NO: 1;
g) an E residue at a position corresponding to position 205 in SEQ ID NO: 1;
h) an S residue at a position corresponding to position 274 in SEQ ID NO: 1;
i) a G residue at a position corresponding to position 275 in SEQ ID NO: 1;
j) an L, T, S, H, M or D residue at a position corresponding to position 289 in SEQ ID NO: 1;
k) a K residue at a position corresponding to position 290 in SEQ ID NO: 1;
l) an F, L, T, V or C residue at a position corresponding to position 291 in SEQ ID NO: 1;
m) an A, Q, C, Y, H, E, F, M, W, T or F residue at a position corresponding to position 292 in SEQ ID NO: 1;
n) an L, V, T, Y, C, F, W, Q, I, N, or M residue at a position corresponding to position 293 in SEQ ID NO: 1; an E, D, N, W, G, V or I residue at a position corresponding to position 295 in SEQ ID NO: 1;
o) an S residue at a position corresponding to position 301 in SEQ ID NO: 1;
p) an E residue at a position corresponding to position 346 in SEQ ID NO: 1;
q) a C residue at a position corresponding to position 368 in SEQ ID NO: 1;
r) a C residue at a position corresponding to position 398 in SEQ ID NO: 1;
s) a W residue at a position corresponding to position 404 in SEQ ID NO: 1;
t) an L, N, W, or T residue at a position corresponding to position 406 in SEQ ID NO: 1;
u) a G residue at a position corresponding to position 407 in SEQ ID NO: 1;
v) an L residue at a position corresponding to position 442 in SEQ ID NO: 1;
w) an I residue at a position corresponding to position 480 in SEQ ID NO: 1;
x) an E residue at a position corresponding to position 494 in SEQ ID NO: 1;
y) a W residue at a position corresponding to position 507 in SEQ ID NO: 1;
z) an A residue at a position corresponding to position 509 in SEQ ID NO: 1;
aa) an L residue at a position corresponding to position 512 in SEQ ID NO: 1;
bb) an F residue at a position corresponding to position 526 of SEQ ID NO: 1;
cc) an N residue at a position corresponding to position 533 of SEQ ID NO: 1; or
dd) any combination thereof.

10. The host cell of any one of claims 1-9, wherein the sesquiterpene synthase comprises the following amino acid substitutions relative to SEQ ID NO: 1:
a) N289H, V292F, G293N, T295V, K404W, F406L, and F512L;
b) N289H, 1291V, V292F, G293Q, T295V, K404W, F406L, and F512L;
c) N289H, F406L, F512L, D191N, and A205E;
d) 1291V, V292F, G293Q, T295V, K404W, F406L, 1507W, and F512L;
e) N289H, V292F, T295V, K404W, F406L, and F512L;
f) N289H, 1291V, V292F, G293N, T295V, K404W, F406L, and F512L;
g) V292F, G293Q, K404W, and F406L;
h) N289H, V292F, G293I, T295V, F406L, 1407G, and F512L;
i) V292F, G293Q, K404W, F406L, 1407G, and F512L;
j) N289H, 1291V, V292F, G293I, T295V, F406L, 1407G, and F512L;
k) V292F, T295V, K404W, F406L, 1507W, and F512L;
l) N289H, V292M, G293I, F406L, F512L, and M480I;
m) N289H, 1291V, V292F, G293Q, T295V, K404W, and F406L;
n) V292F, G293Q, T295V, F406L, and F512L;
o) V292F, T295V, K404W, F406L, and F512L;
p) I291V, V292F, G293I, K404W, F406L, and I507W;
q) V292F, G293I, T295V, F406L, 1407G, and F512L;
r) N289H, V292F, G293I, T295V, F406L, 1507W, and F512L;
s) N289H, 1291V, V292F, G293I, T295V, K404W, F406L, and F512L;
t) N289H, V292F, G293Q, T295V, F406L, 1407G, and F512L;
u) N289H, I291V, V292F, G293N, T295V, K404W, and F406L;
v) N289H, V292F, G293Q, F406L, 1507W, and F512L;
w) I291V, V292F, G293N, T295V, K404W, F406L, 1507W, and F512L;
x) N289H, V292F, G293N, K404W, F406L, 1507W, and F512L;
y) N289H, V292F, G2931, T295V, F406L, 1407G, 1507W, and F512L;
z) N289H, I291V, V292F, G293N, T295V, F406L, and F512L;
aa) I291V, V292F, G293Q, K404W, F406L, 1407G, and F512L;
bb) I291V, V292F, G293Q, T295V, K404W, F406L, 1407G, and F512L;
cc) N289H, V292F, T295V, F406L, and F512L;
dd) N289H, V292F, K404W, F406L, and F512L;
ee) V292F, G293N, T295V, F406L, and F512L;
ff) N289H, 1291V, V292F, G293N, T295V, F406L, 1407G, 1507W, and F512L;
gg) N289H, 1291V, V292F, G293I, T295V, F406L, and F512L;
hh) N289H, 1291V, V292F, G293Q, T295V, F406L, and I507W;
ii) N289H, V292F, G293I, F406L, 1407G, 1507W, and F512L;
jj) N289H, V292F, G293Q, T295V, K404W, F406L, and I507W;
kk) I291V, V292F, G293Q, T295V, F406L, 1507W, and F512L;
ll) S122N, N289H, I291V, V292F, G293Q, T295V, K404W, F406L, and F512L;
mm) P124S, N289H, 1291V, V292F, G293Q, T295V, K404W, F406L, and F512L;
nn) T153S, N289H, I291V, V292F, G293Q, T295V, D346E, K404W, F406L, and F512L
oo) P124S, N289H, 1291V, V292F, G293Q, T295V, K404W, and F406L;
pp) R290K, V292F, T295V, K404W, F406L, and F512L;
qq) N289H, 1291V, V292F, G293Q, T295V, K404W, F406L, Y442L, and F512L;
rr) N289H, V292F, G293N, T295V, K404W, F406L, Y442L, and F512L;
ss) T72I, N289H, 1291V, V292F, G293Q, T295V, K404W, F406L, T494E, and F512L;
tt) T72I, N289H, V292F, T295V, K404W, F406L, T494E, and F512L;
uu) T72I, N289H, V292F, G293I, T295V, F406L, 1407G, Y442L, 1507W, and F512L;
vv) P124S, V292F, T295V, K404W, F406L, 1507W, and F512L;
ww) N289H, V292F, T295V, K404W, F406L, Y442L, and F512L;
xx) N289H, F406L, F512L, and T295V;
yy) N289H, F406L, F512L, T295V, and G274S;
zz) N289H, F406L, F512L, T295V, and V201I; or
aaa) N289H, F406L, F512L, T295V, and I398C.

11. The host cell of any one of claims 1-10, wherein the sesquiterpene synthase comprises a sequence that is at least 90% identical to any one of SEQ ID NOs: 3-39 or 77-92.

12. The host cell of claim 10 or 11, wherein:
a) the sesquiterpene synthase that comprises the amino acid substitutions N289H, V292F, G293N, T295V, K404W, F406L, and F512L relative to SEQ ID NO: 1 comprises the sequence of SEQ ID NO: 3;
b) the sesquiterpene synthase that comprises the amino acid substitutions N289H, 1291V, V292F, G293Q, T295V, K404W, F406L, and F512L relative to SEQ ID NO: 1 comprises the sequence of SEQ ID NO: 4;
c) the sesquiterpene synthase that comprises the amino acid substitutions N289H, F406L, F512L, D191N, and A205E relative to SEQ ID NO: 1 comprises the sequence of SEQ ID NO: 5;
d) the sesquiterpene synthase that comprises the amino acid substitutions I291V, V292F, G293Q, T295V, K404W, F406L, 1507W, and F512L relative to SEQ ID NO: 1 comprises the sequence of SEQ ID NO: 6;
e) the sesquiterpene synthase that comprises the amino acid substitutions N289H, V292F, T295V, K404W, F406L, and F512L relative to SEQ ID NO: 1 comprises the sequence of SEQ ID NO: 7;
f) the sesquiterpene synthase that comprises the amino acid substitutions N289H, 1291V, V292F, G293N, T295V, K404W, F406L, and F512L relative to SEQ ID NO: 1 comprises the sequence of SEQ ID NO: 8;
g) the sesquiterpene synthase that comprises the amino acid substitutions V292F, G293Q, K404W, and F406L relative to SEQ ID NO: 1 comprises the sequence of SEQ ID NO: 9;
h) the sesquiterpene synthase that comprises the amino acid substitutions N289H, V292F, G2931, T295V, F406L, 1407G, and F512L relative to SEQ ID NO: 1 comprises the sequence of SEQ ID NO: 10;
i) the sesquiterpene synthase that comprises the amino acid substitutions V292F, G293Q, K404W, F406L, 1407G, and F512L relative to SEQ ID NO: 1 comprises the sequence of SEQ ID NO: 11;
j) the sesquiterpene synthase that comprises the amino acid substitutions N289H, 1291V, V292F, G293I, T295V, F406L, 1407G, and F512L relative to SEQ ID NO: 1 comprises the sequence of SEQ ID NO: 12;
k) the sesquiterpene synthase that comprises the amino acid substitutions V292F, T295V, K404W, F406L, 1507W, and F512L relative to SEQ ID NO: 1 comprises the sequence of SEQ ID NO: 13;
l) the sesquiterpene synthase that comprises the amino acid substitutions N289H, V292M, G293I, F406L, F512L, and M480I relative to SEQ ID NO: 1 comprises the sequence of SEQ ID NO: 14;
m) the sesquiterpene synthase that comprises the amino acid substitutions N289H, 1291V, V292F, G293Q, T295V, K404W, and F406L relative to SEQ ID NO: 1 comprises the sequence of SEQ ID NO: 15;
n) the sesquiterpene synthase that comprises the amino acid substitutions V292F, G293Q, T295V, F406L, and F512L relative to SEQ ID NO: 1 comprises the sequence of SEQ ID NO: 16;
o) the sesquiterpene synthase that comprises the amino acid substitutions V292F, T295V, K404W, F406L, and F512L relative to SEQ ID NO: 1 comprises the sequence of SEQ ID NO: 17;
p) the sesquiterpene synthase that comprises the amino acid substitutions I291V, V292F, G293I, K404W, F406L, and 1507W relative to SEQ ID NO: 1 comprises the sequence of SEQ ID NO: 18;
q) the sesquiterpene synthase that comprises the amino acid substitutions V292F, G293I, T295V, F406L, 1407G, and F512L relative to SEQ ID NO: 1 comprises the sequence of SEQ ID NO: 19;
r) the sesquiterpene synthase that comprises the amino acid substitutions N289H, V292F, G293I, T295V, F406L, 1507W, and F512L relative to SEQ ID NO: 1 comprises the sequence of SEQ ID NO: 20;
s) the sesquiterpene synthase that comprises the amino acid substitutions N289H, 1291V, V292F, G293I, T295V, K404W, F406L, and F512L relative to SEQ ID NO: 1 comprises the sequence of SEQ ID NO: 21;
t) the sesquiterpene synthase that comprises the amino acid substitutions N289H, V292F, G293Q, T295V, F406L, 1407G, and F512L relative to SEQ ID NO: 1 comprises the sequence of SEQ ID NO: 22;
u) the sesquiterpene synthase that comprises the amino acid substitutions N289H, 1291V, V292F, G293N, T295V, K404W, and F406L relative to SEQ ID NO: 1 comprises the sequence of SEQ ID NO: 23;
v) the sesquiterpene synthase that comprises the amino acid substitutions N289H, V292F, G293Q, F406L, 1507W, and F512L relative to SEQ ID NO: 1 comprises the sequence of SEQ ID NO: 24;
w) the sesquiterpene synthase that comprises the amino acid substitutions 1291V, V292F, G293N, T295V, K404W, F406L, 1507W, and F512L relative to SEQ ID NO: 1 comprises the sequence of SEQ ID NO: 25;
x) the sesquiterpene synthase that comprises the amino acid substitutions N289H, V292F, G293N, K404W, F406L, 1507W, and F512L relative to SEQ ID NO: 1 comprises the sequence of SEQ ID NO: 26;
y) the sesquiterpene synthase that comprises the amino acid substitutions N289H, V292F, G293I, T295V, F406L, 1407G, 1507W, and F512L relative to SEQ ID NO: 1 comprises the sequence of SEQ ID NO: 27;
z) the sesquiterpene synthase that comprises the amino acid substitutions N289H, 1291V, V292F, G293N, T295V, F406L, and F512L relative to SEQ ID NO: 1 comprises the sequence of SEQ ID NO: 28;
aa) the sesquiterpene synthase that comprises the amino acid substitutions 1291V, V292F, G293Q, K404W, F406L, 1407G, and F512L relative to SEQ ID NO: 1 comprises the sequence of SEQ ID NO: 29;
bb) the sesquiterpene synthase that comprises the amino acid substitutions I291V, V292F, G293Q, T295V, K404W, F406L, 1407G, and F512L relative to SEQ ID NO: 1 comprises the sequence of SEQ ID NO: 30;
cc) the sesquiterpene synthase that comprises the amino acid substitutions N289H, V292F, T295V, F406L, and F512L relative to SEQ ID NO: 1 comprises the sequence of SEQ ID NO: 31;
dd) the sesquiterpene synthase that comprises the amino acid substitutions N289H, V292F, K404W, F406L, and F512L relative to SEQ ID NO: 1 comprises the sequence of SEQ ID NO: 32;
ee) the sesquiterpene synthase that comprises the amino acid substitutions V292F, G293N, T295V, F406L, and F512L relative to SEQ ID NO: 1 comprises the sequence of SEQ ID NO: 33;
ff) the sesquiterpene synthase that comprises the amino acid substitutions N289H, 1291V, V292F, G293N, T295V, F406L, 1407G, 1507W, and F512L relative to SEQ ID NO: 1 comprises the sequence of SEQ ID NO: 34;
gg) the sesquiterpene synthase that comprises the amino acid substitutions N289H, 1291V, V292F, G293I, T295V, F406L, and F512L relative to SEQ ID NO: 1 comprises the sequence of SEQ ID NO: 35;
hh) the sesquiterpene synthase that comprises the amino acid substitutions N289H, 1291V, V292F, G293Q, T295V, F406L, and 1507W relative to SEQ ID NO: 1 comprises the sequence of SEQ ID NO: 36;
ii) the sesquiterpene synthase that comprises the amino acid substitutions N289H, V292F, G293I, F406L, 1407G, 1507W, and F512L relative to SEQ ID NO: 1 comprises the sequence of SEQ ID NO: 37;
jj) the sesquiterpene synthase that comprises the amino acid substitutions N289H, V292F, G293Q, T295V, K404W, F406L, and I507W relative to SEQ ID NO: 1 comprises the sequence of SEQ ID NO: 38;
kk) the sesquiterpene synthase that comprises the amino acid substitutions I291V, V292F, G293Q, T295V, F406L, 1507W, and F512L relative to SEQ ID NO: 1 comprises the sequence of SEQ ID NO: 39;
ll) the sesquiterpene synthase that comprises the amino acid substitutions S122N, N289H, 1291V, V292F, G293Q, T295V, K404W, F406L, and F512L relative to SEQ ID NO: 1 comprises the sequence of SEQ ID NO: 84;
mm) the sesquiterpene synthase that comprises the amino acid substitutions P124S, N289H, 1291V, V292F, G293Q, T295V, K404W, F406L, and F512L relative to SEQ ID NO: 1 comprises the sequence of SEQ ID NO: 80;
nn) the sesquiterpene synthase that comprises the amino acid substitutions T153S, N289H, 1291V, V292F, G293Q, T295V, D346E, K404W, F406L, and F512L relative to SEQ ID NO: 1 comprises the sequence of SEQ ID NO: 82;
oo) the sesquiterpene synthase that comprises the amino acid substitutions P124S, N289H, 1291V, V292F, G293Q, T295V, K404W, and F406L relative to SEQ ID NO: 1 comprises the sequence of SEQ ID NO: 81;
pp) the sesquiterpene synthase that comprises the amino acid substitutions R290K, V292F, T295V, K404W, F406L, and F512L relative to SEQ ID NO: 1 comprises the sequence of SEQ ID NO: 83;
qq) the sesquiterpene synthase that comprises the amino acid substitutions N289H, 1291V, V292F, G293Q, T295V, K404W, F406L, Y442L, and F512L relative to SEQ ID NO: 1 comprises the sequence of SEQ ID NO: 78;
rr) the sesquiterpene synthase that comprises the amino acid substitutions N289H, V292F, G293N, T295V, K404W, F406L, Y442L, and F512L relative to SEQ ID NO: 1 comprises the sequence of SEQ ID NO: 77;
ss) the sesquiterpene synthase that comprises the amino acid substitutions T721, N289H, I291V, V292F, G293Q, T295V, K404W, F406L, T494E, and F512L relative to SEQ ID NO: 1 comprises the sequence of SEQ ID NO: 79;
tt) the sesquiterpene synthase that comprises the amino acid substitutions T72I, N289H, V292F, T295V, K404W, F406L, T494E, and F512L relative to SEQ ID NO: 1 comprises the sequence of SEQ ID NO: 88;
uu) the sesquiterpene synthase that comprises the amino acid substitutions T72I, N289H, V292F, G2931, T295V, F406L, 1407G, Y442L, 1507W, and F512L relative to SEQ ID NO: 1 comprises the sequence of SEQ ID NO: 86;
vv) the sesquiterpene synthase that comprises the amino acid substitutions P124S, V292F, T295V, K404W, F406L, 1507W, and F512L relative to SEQ ID NO: 1 comprises the sequence of SEQ ID NO: 85;
ww) the sesquiterpene synthase that comprises the amino acid substitutions N289H, V292F, T295V, K404W, F406L, Y442L, and F512L relative to SEQ ID NO: 1 comprises the sequence of SEQ ID NO: 87;
xx) the sesquiterpene synthase that comprises the amino acid substitutions N289H, F406L, F512L, and T295V relative to SEQ ID NO: 1 comprises the sequence of SEQ ID NO: 89;
yy) the sesquiterpene synthase that comprises the amino acid substitutions N289H, F406L, F512L, T295V, and G274S relative to SEQ ID NO: 1 comprises the sequence of SEQ ID NO: 90;
zz) the sesquiterpene synthase that comprises the amino acid substitutions N289H, F406L, F512L, T295V, and V201I relative to SEQ ID NO: 1 comprises the sequence of SEQ ID NO: 91; or
aaa) the sesquiterpene synthase that comprises the amino acid substitutions N289H, F406L, F512L, T295V, and I398C relative to SEQ ID NO: 1 comprises the sequence of SEQ ID NO: 92.

13. The host cell of any one of claims 1-12, wherein the heterologous nucleic acid comprises a sequence that is at least 90% identical to any one of SEQ ID NOs: 40-76 or 93-109.

14. The host cell of any one of claims 1-8, wherein the sesquiterpene synthase further comprises at least one amino acid substitution at a position corresponding to position 294, 296, 297, 403, 444, 515, and/or 525 in SEQ ID NO: 1.

15. The host cell of claim 14, wherein the sesquiterpene synthase comprises:
a) an L, C, Y, V, A, or S residue at a position corresponding to position 294 in SEQ ID NO: 1;
b) an L, A, proline (P), Y, N, F or R residue at a position corresponding to position 296 in SEQ ID NO: 1;
c) an E, Y, I, lysine (K), M, or H residue at a position corresponding to position 297 in SEQ ID NO: 1;
d) an M, Q, N, S, T, A, E, H, C, or V residue at a position corresponding to position 403 in SEQ ID NO: 1;
e) an A or N residue at a position corresponding to position 444 in SEQ ID NO: 1;
f) an H, A, E, or Q residue at a position corresponding to position 515 in SEQ ID NO: 1;
g) an H, C, L, or N residue at a position corresponding to position 525 in SEQ ID NO: 1; or
h) any combination thereof.

16. The host cell of any one of claims 1-15, wherein the host cell is capable of producing more alpha-guaiene than a host cell that expresses a sesquiterpene synthase that comprises the sequence of SEQ ID NO: 1.

17. The host cell of any one of claims 1-16, wherein the host cell produces more alpha-guaiene than delta-guaiene.

18. The host cell of any one of claims 1-4, wherein the sesquiterpene synthase comprises:
a) one or more amino acid substitutions in a first region of the active site relative to SEQ ID NO: 1, wherein the one or more amino acid substitutions in the first region are at positions that correspond to positions 295, 291, 406, 512, and/or 519 of SEQ ID NO: 1, and the one or more amino acid substitutions in the first region each comprises a residue with a smaller side chain than that of the amino acid at positions 295, 291, 406, 512, and/or 519 in SEQ ID NO: 1;
b) an amino acid substitution in a second region of the active site relative to SEQ ID NO: 1, wherein the amino acid substitution in the second region corresponds to position 292 of SEQ ID NO: 1, and wherein the amino acid substitution in the second region comprises a larger side chain than that of the amino acid at position 292 in SEQ ID NO: 1; or
c) any combination thereof.

19. The host cell of claim 18, wherein the side chain in (a) is hydrophobic.

20. The host cell of claim 18 or 19, wherein the sesquiterpene synthase comprises:
a) a V residue at a position corresponding to position 295 in SEQ ID NO: 1;
b) a C residue at a position corresponding to position 291 in SEQ ID NO: 1;
c) a L residue at a position corresponding to position 406 in SEQ ID NO: 1;
d) a L residue at a position corresponding to position 512 in SEQ ID NO: 1;
e) a F residue at a position corresponding to position 292 in SEQ ID NO: 1; or
f) any combination thereof.

21. The host cell of any one of claims 1-4, wherein the sesquiterpene synthase further comprises one or more amino acid substitutions relative to SEQ ID NO: 1 at one or more positions corresponding to position 294, 296, 297, 403, 444, 515, and/or 525 in SEQ ID NO: 1.

22. The host cell of claim 21, wherein the sesquiterpene synthase comprises:
a) a L, T, S, D, or M residue at a position corresponding to position 289 in SEQ ID NO: 1;
b) an F, L, T, or C residue at a position corresponding to position 291 in SEQ ID NO: 1;
c) an A, Q, C, Y, H, E, F, T, or W residue at a position corresponding to position 292 in SEQ ID NO: 1;
d) a L, V, T, Y, C, F, W, or M residue at a position corresponding to position 293 in SEQ ID NO: 1;
e) a L, C, Y, V, A, or S residue at a position corresponding to position 294 in SEQ ID NO: 1;
f) an E, D, N, W, G, or I residue at a position corresponding to position 295 in SEQ ID NO: 1;
g) a L, A, P, Y, N, arginine (R), or F residue at a position corresponding to position 296 in SEQ ID NO: 1;
h) an E, Y, I, K, M, or H residue at a position corresponding to position 297 in SEQ ID NO: 1;
i) a M, Q, N, V, C, H, E, A, T, or S residue at a position corresponding to position 403 in SEQ ID NO: 1;
j) a L, T, W, or N residue at a position corresponding to position 406 in SEQ ID NO: 1;
k) an A or N residue at a position corresponding to position 444 in SEQ ID NO: 1;
l) an H, A, E, or Q residue at a position corresponding to position 515 in SEQ ID NO: 1;
m) an H, C, L, or N residue at a position corresponding to position 525 in SEQ ID NO: 1; or
n) any combination thereof.

23. The host cell of claim 22, wherein the sesquiterpene synthase does not comprise:
a) a Q, C, V, F, A, I, H, G, W, or Y residue at a position corresponding to position 289 in SEQ ID NO: 1;
b) a V, M, or A residue at a position corresponding to position 291 in SEQ ID NO: 1;
c) an I, M, S, L, G, or N residue at a position corresponding to position 292 in SEQ ID NO: 1;
d) an I, Q, S, N, or E residue at a position corresponding to position 293 in SEQ ID NO: 1;
e) a L, V, A, or S residue at a position corresponding to position 295 in SEQ ID NO: 1;
f) a K or Q residue at a position corresponding to position 296 in SEQ ID NO: 1;
g) a T or A residue at a position corresponding to position 297 in SEQ ID NO: 1;
h) a P, F, I, L, G, or D residue at a position corresponding to position 403 in SEQ ID NO: 1;
i) a G, S, A, I, Y, M, H, V, Q, or C residue at a position corresponding to position 406 in SEQ ID NO: 1;
j) an H residue at a position corresponding to position 444 in SEQ ID NO: 1;
k) a M, C, F, G, N, S, or I residue at a position corresponding to position 515 in SEQ ID NO: 1; or
l) any combination thereof.

24. A non-naturally occurring sesquiterpene synthase, wherein the sequence of the sesquiterpene synthase comprises one or more amino acid substitutions relative to SEQ ID NO: 1, wherein at least one of the amino acid substitutions is at a position corresponding to position 122, 274, 275, 293, 295, 301, 368, 398, 404, 407, 507, 515, 525, and/or 533 in SEQ ID NO: 1.

25. The non-naturally occurring sesquiterpene synthase of claim 24, wherein the sequence of the sesquiterpene synthase further comprises at least one amino acid substitution at a position corresponding to position 72, 124, 153, 191, 201, 205, 289, 290, 291, 292, 346, 406, 442, 480, 494, 509, 512, and/or 526 in SEQ ID NO: 1.

26. A non-naturally occurring sesquiterpene synthase, wherein the sequence of the sesquiterpene synthase comprises two or more amino acid substitutions relative to SEQ ID NO: 1, wherein:
(i) at least one amino acid substitution is at a position corresponding to position 122, 274, 275, 293, 295, 301, 368, 398, 404, 407, 507, 515, 525, and/or 533 in SEQ ID NO: 1; and
(ii) at least one amino acid substitution is at a position corresponding to position 72, 124, 153, 191, 201, 205, 289, 290, 291, 292, 346, 406, 442, 480, 494, 509, 512, and/or 526 in SEQ ID NO: 1.

27. A non-naturally occurring sesquiterpene synthase, wherein the sesquiterpene synthase comprises an amino acid sequence that is at least 90% identical to the sequence of SEQ ID NO: 1, wherein the sesquiterpene synthase comprises an amino acid sequence having two or more amino acid substitutions relative to SEQ ID NO: 1, and wherein the two or more amino acid substitutions are at positions corresponding to position 72, 122, 124, 153, 191, 201, 205, 274, 275, 289, 290, 291, 292, 293, 295, 301, 346, 368, 398, 404, 406, 407, 442, 480, 494, 507, 509, 512, 526 and/or 533 in SEQ ID NO: 1, wherein the sesquiterpene synthase is capable of producing sesquiterpene products, and wherein at least 50% of sesquiterpene products produced by the sesquiterpene synthase is alpha-guaiene.

28. The non-naturally occurring sesquiterpene synthase of any one of claims 24-27, wherein the amino acid sequence of the sesquiterpene synthase is at least 90% identical to any one of SEQ ID NOs: 3-39 or 77-92.

29. A non-naturally occurring nucleic acid encoding a sesquiterpene synthase, wherein the non-naturally occurring nucleic acid comprises a sequence that is at least 90% identical to SEQ ID NOs: 40-76 or 93-109.

30. A method of producing a sesquiterpene comprising culturing the host cell of any one of claims 1-23 in culture medium in the presence of an FPP substrate and optionally isolating or recovering the sesquiterpene from the host cell and/or the culture medium.

31. The method of claim 30, wherein the sesquiterpene is alpha-guaiene.

32. The method of claim 30, wherein the sesquiterpene is aciphyllene.

33. The method of claim 30 or 31, wherein at least 50% of the sesquiterpenes isolated or recovered from the host cell or culture medium is alpha-guaiene.

34. The method of claim 33, wherein at least 15% of the sesquiterpenes isolated or recovered from the host cell or culture medium is aciphyllene.

35. The method of claim 33, wherein the alpha-guaiene is recovered from the culture medium.

36. The method of claim 34, wherein the aciphyllene is recovered from the culture medium.

37. The method of any one of claims 30-34, further comprising obtaining a composition comprising alpha-guaiene.

38. The method of any one of claims 30-37, further comprising obtaining a composition comprising aciphyllene.

39. A sesquiterpene obtainable from the host cell of any one of claims 1-23 or from the method according to any one of claims 30-37.

40. The sesquiterpene of claim 39, wherein the sesquiterpene is alpha-guaiene.

41. The sesquiterpene of claim 39, wherein the sesquiterpene is aciphyllene.

42. A culture medium comprising the sesquiterpene according to any one of claims 39-41.

43. A composition comprising the sesquiterpene according to any one of claims 39-41.

44. The composition of claim 43, wherein the sesquiterpene is alpha-guaiene.

45. The composition of claim 43, wherein the sesquiterpene is aciphyllene.

46. A composition comprising (a) a sesquiterpene, wherein at least 50% of the sesquiterpene is alpha-guaiene, and (b) one or more additional components, wherein the one or more additional components include fermentation medium, cell culture supernatant and/or a hydrophobic overlay.

47. The composition of claim 46, wherein at least 15% of the sesquiterpene is aciphyllene.

48. The composition of claim 46 wherein the alpha-guaiene is produced using a microbial host cell.

49. The composition of claim 46 or 48, wherein the alpha-guaiene is produced using an in-vitro or an in-vivo system.

50. The composition of any one of claims 46-49, wherein about 50% to about 90% of the sesquiterpene is alpha-guaiene.

51. The composition of any one of claims 46-50, further comprising delta-guaiene.

52. The composition of any one of claims 46-51, further comprising aciphyllene.

53. The composition of claim 51, wherein about 50% to about 10% of the sesquiterpene is alpha-guaiene.

54. The composition of any one of claims 46-53, further comprising one or more non-terpene components or one or more additional terpene components.

55. The composition of claim 54, wherein the one or more non-terpene components include FPP.

56. The composition of claim 54, wherein the one or more additional terpene components include: delta-guaiene, beta-guaiene, gamma guaiene, germacrene A, aciphyllene, and/or alpha-humulene, as determined by GC.

57. A method of making the composition of any one of claims 46-56 comprising culturing a host cell that comprises a heterologous polynucleotide encoding a sesquiterpene synthase, wherein the sesquiterpene synthase comprises one or more amino acid substitutions relative to SEQ ID NO: 1, wherein at least one of the amino acid substitutions is at a position corresponding to position 122, 274, 275, 293, 295, 301, 368, 398, 404, 407, 507, 515, 525, and/or 533 in SEQ ID NO: 1.

58. The method of claim 57, wherein the sequence of the sesquiterpene synthase further comprises at least one amino acid substitution at a position corresponding to position 72, 124, 153, 191, 201, 205, 289, 290, 291, 292, 346, 406, 442, 480, 494, 509, 512, and/or 526 in SEQ ID NO: 1.

59. A method of making the composition of any one of claims 46-56 comprising culturing a host cell that comprises a heterologous polynucleotide encoding a sesquiterpene synthase, wherein the sequence of the sesquiterpene synthase comprises two or more amino acid substitutions relative to SEQ ID NO: 1, wherein:
(i) at least one amino acid substitution is at a position corresponding to position 122, 274, 275, 293, 295, 301, 368, 398, 404, 407, 507, 515, 525, and/or 533 in SEQ ID NO: 1; and
(ii) at least one amino acid substitution is at a position corresponding to position 72, 124, 153, 191, 201, 205, 289, 290, 291, 292, 346, 406, 442, 480, 494, 509, 512, and/or 526 in SEQ ID NO: 1.

60. A method of making the composition of any one of claims 46-56 comprising culturing a host cell that comprises a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, wherein the two or more amino acid substitutions are at positions corresponding to position 72, 122, 124, 153, 191, 201, 205, 274, 275, 289, 290, 291, 292, 293, 295, 301, 346, 368, 398, 404, 406, 407, 442, 480, 494, 507, 509, 512, 526 and/or 533 in SEQ ID NO: 1.

61. A host cell that comprises a heterologous polynucleotide encoding a sesquiterpene synthase, wherein the sequence of the sesquiterpene synthase comprises one or more amino acid substitutions relative to SEQ ID NO: 1, and wherein at least one of the amino acid substitutions is at a position corresponding to position 44, 212, 217, 293, 295, 404, 515, and/or 542 in SEQ ID NO: 1.

62. The host cell of claim 61, wherein the sequence of the sesquiterpene synthase further comprises at least one amino acid substitution at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

63. A host cell that comprises a heterologous polynucleotide encoding a sesquiterpene synthase, wherein the sequence of the sesquiterpene synthase comprises two or more amino acid substitutions relative to SEQ ID NO: 1, wherein:
(i) at least one of the amino acid substitutions is at a position corresponding to position 44, 212, 217, 293, 295, 404, 515, and/or 542 in SEQ ID NO: 1; and
(ii) at least one of the amino acid substitutions is at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

64. The host cell of any one of claims 61-63, wherein the host cell is capable of producing sesquiterpene products, and wherein at least 50% of the total sesquiterpene products produced by the host cell is alpha-guaiene.

65. The host cell of any one of claims 61-64, wherein at least 15% of the total sesquiterpene products produced by the host cell is aciphyllene.

66. A host cell that comprises a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, wherein the two or more amino acid substitutions are at positions corresponding to position 23, 44, 72, 86, 111, 118, 134, 147, 188, 201, 212, 217, 224, 252, 255, 289, 290, 291, 292, 293, 295, 346, 381, 390, 404, 406, 419, 433, 442, 443, 444, 448, 458, 467, 494, 499, 512, 515, 516, 519, or 542 in SEQ ID NO: 1, wherein the host cell is capable of producing sesquiterpene products, and wherein at least 50% of the total sesquiterpene products produced by the host cell is alpha-guaiene.

67. The host cell of any one of claims 61-66, wherein the sesquiterpene synthase comprises 4, 5, 6, 7, 8, 9, 10, 11, 12, 13, 14, 15, 16, 17, 18, 19, 20, 21, 22, 23, 24, 25, 26, 27, or more than 27 amino acid substitutions relative to SEQ ID NO: 1.

68. The host cell of any one of claims 61-67, wherein the sesquiterpene synthase comprises:
(a) an aspartate (D) residue at a position corresponding to position 23 in SEQ ID NO: 1;
(b) a valine (V) residue at a position corresponding to position 44 in SEQ ID NO: 1;
(c) an isoleucine (I) residue at a position corresponding to position 72 in SEQ ID NO: 1;
(d) a glutamate (E) residue at a position corresponding to position 86 in SEQ ID NO: 1;
(e) a leucine (L) residue at a position corresponding to position 111 in SEQ ID NO: 1;
(f) a glutamine (Q) residue at a position corresponding to position 118 in SEQ ID NO: 1;
(g) a D residue at a position corresponding to position 134 in SEQ ID NO: 1;
(h) a V residue at a position corresponding to position 147 in SEQ ID NO: 1;
(i) an L residue at a position corresponding to position 147 in SEQ ID NO: 1;
(j) a Q residue at a position corresponding to position 188 in SEQ ID NO: 1;
(k) a serine(S) residue at a position corresponding to position 201 in SEQ ID NO: 1;
(l) a phenylalanine (F) residue at a position corresponding to position 212 in SEQ ID NO: 1;
(m) an E residue at a position corresponding to position 217 in SEQ ID NO: 1;
(n) an L residue at a position corresponding to position 224 in SEQ ID NO: 1;
(o) an L residue at a position corresponding to position 252 in SEQ ID NO: 1;
(p) an alanine (A) residue at a position corresponding to position 255 in SEQ ID NO: 1;
(q) a lysine (K) residue at a position corresponding to position 289 in SEQ ID NO: 1;
(r) a histidine (H) residue at a position corresponding to position 290 in SEQ ID NO: 1;
(s) a V residue at a position corresponding to position 291 in SEQ ID NO: 1;
(t) a cysteine (C) residue at a position corresponding to position 292 in SEQ ID NO: 1;
(u) an F residue at a position corresponding to position 292 in SEQ ID NO: 1;
(v) an asparagine (N) residue at a position corresponding to position 293 in SEQ ID NO: 1;
(w) a Q residue at a position corresponding to position 293 in SEQ ID NO: 1;
(x) an I residue at a position corresponding to position 295 in SEQ ID NO: 1;
(y) a V residue at a position corresponding to position 295 in SEQ ID NO: 1;
(z) an E residue at a position corresponding to position 346 in SEQ ID NO: 1;
(aa) a tryptophan (W) residue at a position corresponding to position 381 in SEQ ID NO: 1;
(bb) an F residue at a position corresponding to position 390 in SEQ ID NO: 1;
(cc) a W residue at a position corresponding to position 404 in SEQ ID NO: 1;
(dd) an L residue at a position corresponding to position 406 in SEQ ID NO: 1;
(ee) an S residue at a position corresponding to position 419 in SEQ ID NO: 1;
(ff) an I residue at a position corresponding to position 433 in SEQ ID NO: 1;
(gg) an L residue at a position corresponding to position 433 in SEQ ID NO: 1;
(hh) an L residue at a position corresponding to position 442 in SEQ ID NO: 1;
(ii) a W residue at a position corresponding to position 443 in SEQ ID NO: 1;
(jj) a threonine (T) residue at a position corresponding to position 443 in SEQ ID NO: 1;
(kk) an N residue at a position corresponding to position 444 in SEQ ID NO: 1;
(ll) a G residue at a position corresponding to position 448 in SEQ ID NO: 1;
(mm) an L residue at a position corresponding to position 458 in SEQ ID NO: 1;
(nn) a V residue at a position corresponding to position 458 in SEQ ID NO: 1;
(00) a K residue at a position corresponding to position 467 in SEQ ID NO: 1;
(pp) an E residue at a position corresponding to position 494 in SEQ ID NO: 1;
(qq) an N residue at a position corresponding to position 499 in SEQ ID NO: 1;
(rr) an L residue at a position corresponding to position 512 in SEQ ID NO: 1;
(ss) a methionine (M) residue at a position corresponding to position 515 in SEQ ID NO: 1;
(tt) an A residue at a position corresponding to position 516 in SEQ ID NO: 1;
(uu) a V residue at a position corresponding to position 516 in SEQ ID NO: 1;
(vv) an L residue at a position corresponding to position 519 in SEQ ID NO: 1;
(ww) a V residue at a position corresponding to position 542 in SEQ ID NO: 1; or
(xx) any combination thereof.

69. The host cell of any one of claims 61-68, wherein the sesquiterpene synthase comprises the following amino acid substitutions relative to SEQ ID NO: 1:
(a) T72I, N289H, R290K, 1291V, V292F, G293Q, T295V, K404W, F406L, Y442L, T494E, and F512L;
(b) L44V, T72I, N289H, I291V, V292F, G293Q, T295V, K404W, F406L, Y442L, and F512L;
(c) L44V, T72I, T86E, G118Q, T134D, W147L, Q212F, Q217E, S224L, F252L, P255A, N289H, R290K, 1291V, V292F, G293Q, T295V, D346E, Y390F, K404W, F406L, L419S, Y442L, T458V, R467K, F512L, and R542V;
(d) T23D, L44V, T72I, N289H, I291V, V292F, G293Q, T295V, K404W, F406L, Y442L, and F512L;
(e) V201S, N289H, I291V, V292F, G293N, T295V, Y381W, K404W, F406L, T494E, and F512L;
(f) V201S, V292C, T295I, and D444N;
(g) V201S, V292C, T295I, D444N, and L516V;
(h) L44V, T72I, V201S, V292C, T295I, D444N, and L516V;
(i) L44V, T72I, V201S, V292C, T295I, D444N, and L516A;
(j) L44V, T72I, V201S, V292C, T295I, 1443W, and D444N;
(k) L44V, T72I, V201S, V292C, T295I, D444N, and M519L;
(l) W147V, C188Q, V201S, S224L, V292C, T295I, and D444N;
(m) L44V, T72I, V111L, V201S, S224L, V292C, T295I, 1443T, D444N, T448G, and M519L;
(n) L44V, T72I, V111L, V201S, V292C, T295I, V433I, 1443T, D444N, T448G, D499N, and L516V;
(o) L44V, T72I, V201S, V292C, T295I, D444N, L516V, and M519L;
(p) L44V, T72I, V201S, V292C, T295I, D444N, V515M, and L516V;
(q) V111L, V201S, S224L, V292C, T2951, 1443T, D444N, and L516V;
(r) V201S, V292C, T295I, D444N, and L516V;
(s) V201S, V292C, T295I, D444N, T458L, and L516A;
(t) V201S, V292C, T2951, 1443T, D444N, and L516V;
(u) V201S, V292C, T295I, V433I, D444N, and L516V; or
(v) V201S, V292C, T295I, V433L, D444N, and L516V.

70. The host cell of any one of claims 61-69, wherein the sesquiterpene synthase comprises a sequence that is at least 90% identical to any one of SEQ ID NOs: 110-131.

71. The host cell of claim 60 or 70, wherein:
(a) the sesquiterpene synthase that comprises the amino acid substitutions T72I, N289H, R290K, 1291V, V292F, G293Q, T295V, K404W, F406L, Y442L, T494E, and F512L relative to SEQ ID NO: 1 comprises the sequence of SEQ ID NO: 110;
(b) the sesquiterpene synthase that comprises the amino acid substitutions L44V, T721, N289H, I291V, V292F, G293Q, T295V, K404W, F406L, Y442L, and F512L relative to SEQ ID NO: 1 comprises the sequence of SEQ ID NO: 111;
(c) the sesquiterpene synthase that comprises the amino acid substitutions L44V, T721, T86E, G118Q, T134D, W147L, Q212F, Q217E, S224L, F252L, P255A, N289H, R290K, 1291V, V292F, G293Q, T295V, D346E, Y390F, K404W, F406L, L419S, Y442L, T458V, R467K, F512L, and R542V relative to SEQ ID NO: 1 comprises the sequence of SEQ ID NO: 112;
(d) the sesquiterpene synthase that comprises the amino acid substitutions T23D, L44V, T721, N289H, 1291V, V292F, G293Q, T295V, K404W, F406L, Y442L, and F512L relative to SEQ ID NO: 1 comprises the sequence of SEQ ID NO: 113;
(e) the sesquiterpene synthase that comprises the amino acid substitutions V201S, N289H, 1291V, V292F, G293N, T295V, Y381W, K404W, F406L, T494E, and F512L relative to SEQ ID NO: 1 comprises the sequence of SEQ ID NO: 114;
(f) the sesquiterpene synthase that comprises the amino acid substitutions V201S, V292C, T2951, and D444N relative to SEQ ID NO: 1 comprises the sequence of SEQ ID NO: 115;
(g) the sesquiterpene synthase that comprises the amino acid substitutions V201S, V292C, T295I, D444N, and L516V relative to SEQ ID NO: 1 comprises the sequence of SEQ ID NO: 116;
(h) the sesquiterpene synthase that comprises the amino acid substitutions L44V, T72I, V201S, V292C, T295I, D444N, and L516V relative to SEQ ID NO: 1 comprises the sequence of SEQ ID NO: 117;
(i) the sesquiterpene synthase that comprises the amino acid substitutions L44V, T72I, V201S, V292C, T295I, D444N, and L516A relative to SEQ ID NO: 1 comprises the sequence of SEQ ID NO: 118;
(j) the sesquiterpene synthase that comprises the amino acid substitutions L44V, T72I, V201S, V292C, T2951, 1443W, and D444N relative to SEQ ID NO: 1 comprises the sequence of SEQ ID NO: 119;
(k) the sesquiterpene synthase that comprises the amino acid substitutions L44V, T72I, V201S, V292C, T2951, D444N, and M519L relative to SEQ ID NO: 1 comprises the sequence of SEQ ID NO: 120;
(1) the sesquiterpene synthase that comprises the amino acid substitutions W147V, C188Q, V201S, S224L, V292C, T295I, and D444N relative to SEQ ID NO: 1 comprises the sequence of SEQ ID NO: 121;
(m) the sesquiterpene synthase that comprises the amino acid substitutions L44V, T72I, V111L, V201S, S224L, V292C, T2951, 1443T, D444N, T448G, and M519L relative to SEQ ID NO: 1 comprises the sequence of SEQ ID NO: 122;
(n) the sesquiterpene synthase that comprises the amino acid substitutions L44V, T72I, V111L, V201S, V292C, T295I, V4331, 1443T, D444N, T448G, D499N, and L516V relative to SEQ ID NO: 1 comprises the sequence of SEQ ID NO: 123;
(o) the sesquiterpene synthase that comprises the amino acid substitutions L44V, T72I, V201S, V292C, T2951, D444N, L516V, and M519L relative to SEQ ID NO: 1 comprises the sequence of SEQ ID NO: 124;
(p) the sesquiterpene synthase that comprises the amino acid substitutions L44V, T72I, V201S, V292C, T295I, D444N, V515M, and L516V relative to SEQ ID NO: 1 comprises the sequence of SEQ ID NO: 125;
(q) the sesquiterpene synthase that comprises the amino acid substitutions V111L, V201S, S224L, V292C, T2951, 1443T, D444N, and L516V relative to SEQ ID NO: 1 comprises the sequence of SEQ ID NO: 126;
(r) the sesquiterpene synthase that comprises the amino acid substitutions V201S, V292C, T295I, D444N, and L516V relative to SEQ ID NO: 1 comprises the sequence of SEQ ID NO: 127;
(s) the sesquiterpene synthase that comprises the amino acid substitutions V201S, V292C, T295I, D444N, T458L, and L516A relative to SEQ ID NO: 1 comprises the sequence of SEQ ID NO: 128;
(t) the sesquiterpene synthase that comprises the amino acid substitutions V201S, V292C, T2951, 1443T, D444N, and L516V relative to SEQ ID NO: 1 comprises the sequence of SEQ ID NO: 129;
(u) the sesquiterpene synthase that comprises the amino acid substitutions V201S, V292C, T295I, V433I, D444N, and L516V relative to SEQ ID NO: 1 comprises the sequence of SEQ ID NO: 130; or
(v) the sesquiterpene synthase that comprises the amino acid substitutions V201S, V292C, T295I, V433L, D444N, and L516V relative to SEQ ID NO: 1 comprises the sequence of SEQ ID NO: 131.

72. The host cell of any one of claims 61-71, wherein the heterologous nucleic acid comprises a sequence that is at least 90% identical to any one of SEQ ID NOs: 133-154.

73. The host cell of any one of claims 61-72, wherein the host cell is capable of producing more alpha-guaiene than a host cell that expresses a sesquiterpene synthase that comprises the sequence of SEQ ID NO: 1.

74. The host cell of any one of claims 61-73, wherein the host cell produces more alpha-guaiene than delta-guaiene.

75. The host cell of any one of claims 61-74, wherein the host cell produces more aciphyllene than delta-guaiene.

76. A non-naturally occurring sesquiterpene synthase, wherein the sequence of the sesquiterpene synthase comprises one or more amino acid substitutions relative to SEQ ID NO: 1, wherein at least one of the amino acid substitutions is at a position corresponding to position 44, 212, 217, 293, 295, 404, 515, and/or 542 in SEQ ID NO: 1.

77. The non-naturally occurring sesquiterpene synthase of claim 76, wherein the sequence of the sesquiterpene synthase further comprises at least one amino acid substitution at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

78. A non-naturally occurring sesquiterpene synthase, wherein the sequence of the sesquiterpene synthase comprises two or more amino acid substitutions relative to SEQ ID NO: 1, wherein:
(i) at least one amino acid substitution is at a position corresponding to position 44, 212, 217, 293, 295, 404, 515, and/or 542 in SEQ ID NO: 1; and
(ii) at least one amino acid substitution is at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

79. A non-naturally occurring sesquiterpene synthase, wherein the sesquiterpene synthase comprises an amino acid sequence that is at least 90% identical to the sequence of SEQ ID NO: 1, wherein the sesquiterpene synthase comprises an amino acid sequence having two or more amino acid substitutions relative to SEQ ID NO: 1, and wherein the two or more amino acid substitutions are at positions corresponding to position 23, 44, 72, 86, 111, 118, 134, 147, 188, 201, 212, 217, 224, 252, 255, 289, 290, 291, 292, 293, 295, 346, 381, 390, 404, 406, 419, 433, 442, 443, 444, 448, 458, 467, 494, 499, 512, 515, 516, 519, or 542 in SEQ ID NO: 1, wherein the sesquiterpene synthase is capable of producing sesquiterpene products, and wherein at least 50% of sesquiterpene products produced by the sesquiterpene synthase is alpha-guaiene.

80. The non-naturally occurring sesquiterpene synthase of any one of claims 76-79, wherein the amino acid sequence of the sesquiterpene synthase is at least 90% identical to any one of SEQ ID NOs: 110-131.

81. A non-naturally occurring nucleic acid encoding a sesquiterpene synthase, wherein the non-naturally occurring nucleic acid comprises a sequence that is at least 90% identical to SEQ ID NOs: 133-154.

82. A method of producing a sesquiterpene comprising culturing the host cell of any one of claims 61-75 in culture medium in the presence of an FPP substrate and optionally isolating or recovering the sesquiterpene from the host cell and/or the culture medium.

83. The method of claim 82, wherein the sesquiterpene is alpha-guaiene.

84. The method of claim 82, wherein the sesquiterpene is aciphyllene.

85. The method of claim 82 or 83, wherein 50% of the sesquiterpenes isolated or recovered from the host cell or culture medium is alpha-guaiene.

86. The method of any one of claims 82-84, wherein at least 15% of the sesquiterpenes isolated or recovered from the host cell or culture medium is aciphyllene.

87. The method of claim 85, wherein the alpha-guaiene is recovered from the culture medium.

88. The method of any one of claims 82-87, wherein the aciphyllene is recovered from the culture medium.

89. The method of any one of claims 82-87, further comprising obtaining a composition comprising alpha-guaiene.

90. The method of any one of claims 82-89, further comprising obtaining a composition comprising aciphyllene.

91. A sesquiterpene obtainable from the host cell of any one of claims 61-74 or from the method according to any one of claims 82-90.

92. The sesquiterpene of claim 91, wherein the sesquiterpene is alpha-guaiene.

93. The sesquiterpene of claim 91, wherein the sesquiterpene is aciphyllene.

94. A culture medium comprising the sesquiterpene according to claim 91 or claim 92.

95. A composition comprising the sesquiterpene according to claim 91 or 92.

96. The composition of claim 95, wherein the sesquiterpene is alpha-guaiene.

97. The composition of claim 95, wherein the sesquiterpene is aciphyllene.

98. A composition comprising (a) a sesquiterpene, wherein at least 50% of the sesquiterpene is alpha-guaiene, and (b) one or more additional components, wherein the one or more additional components include fermentation medium, cell culture supernatant and/or a hydrophobic overlay.

99. The composition of claim 98, wherein at least 15% of the sesquiterpene is aciphyllene.

100. The composition of claim 98 wherein the alpha-guaiene is produced using a microbial host cell.

101. The composition of claim 98 or 100, wherein the alpha-guaiene is produced using an in-vitro or an in-vivo system.

102. The composition of any one of claims 98-101, wherein about 50% to about 90% of the sesquiterpene is alpha-guaiene.

103. The composition of any one of claims 98-102, further comprising delta-guaiene.

104. The composition of any one of claims 98-102, further comprising aciphyllene.

105. The composition of claim 103, wherein about 50% to about 10% of the sesquiterpene is alpha-guaiene.

106. The composition of claim 105, wherein at least 15% of the sesquiterpene is aciphyllene.

107. The composition of any one of claims 98-105 further comprising one or more non-terpene components or one or more additional terpene components.

108. The composition of claim 107, wherein the one or more non-terpene components include FPP.

109. The composition of claim 107, wherein the one or more additional terpene components include: delta-guaiene, beta-guaiene, gamma guaiene, germacrene A, aciphyllene, and/or alpha-humulene, as determined by GC.

110. A method of making the composition of any one of claims 98-109 comprising culturing a host cell that comprises a heterologous polynucleotide encoding a sesquiterpene synthase, wherein the sesquiterpene synthase comprises one or more amino acid substitutions relative to SEQ ID NO: 1, wherein at least one of the amino acid substitutions is at a position corresponding to position 44, 212, 217, 293, 295, 404, 448, 515, and/or 542 in SEQ ID NO: 1.

111. The method of claim 110, wherein the sequence of the sesquiterpene synthase further comprises at least one amino acid substitution at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

112. A method of making the composition of any one of claims 98-109 comprising culturing a host cell that comprises a heterologous polynucleotide encoding a sesquiterpene synthase, wherein the sequence of the sesquiterpene synthase comprises two or more amino acid substitutions relative to SEQ ID NO: 1, wherein:
(i) at least one amino acid substitution is at a position corresponding to position 44, 212, 217, 293, 295, 404, 515, and/or 542 in SEQ ID NO: 1; and
(ii) at least one amino acid substitution is at a position corresponding to position 23, 72, 86, 111, 118, 134, 147, 188, 201, 224, 252, 255, 289, 290, 291, 292, 346, 381, 390, 406, 419, 433, 442, 443, 444, 448, 458, 467, 494, 499, 512, 516, and/or 519 in SEQ ID NO: 1.

113. A method of making the composition of any one of claims 98-109 comprising culturing a host cell that comprises a heterologous polynucleotide encoding a sesquiterpene synthase, wherein the sesquiterpene synthase comprises an amino acid sequence that is at least 90% identical to the sequence of SEQ ID NO: 1, wherein the sequence of the sesquiterpene synthase comprises two or more amino acid substitutions relative to SEQ ID NO: 1, wherein the two or more amino acid substitutions are at positions corresponding to position 23, 44, 72, 86, 111, 118, 134, 147, 188, 201, 212, 217, 224, 252, 255, 289, 290, 291, 292, 293, 295, 346, 381, 390, 404, 406, 419, 433, 442, 443, 444, 448, 458, 467, 494, 499, 512, 515, 516, 519, or 542 in SEQ ID NO: 1.
